SEQUENCE LISTING (10 sequences)

SEQ ID NO: 1 - DNA, 23 bp, Artificial Sequence
  Description of Artificial Sequence Synthetic primer
ttccacgatgtgccgtgccttta

SEQ ID NO: 2 - DNA, 25 bp, Artificial Sequence
  Description of Artificial Sequence Synthetic primer
ggcacataggaggagtgtggtcaca

SEQ ID NO: 3 - DNA, 26 bp, Artificial Sequence
  Description of Artificial Sequence Synthetic primer
ttccaggatgtggctccctgctcttg

SEQ ID NO: 4 - DNA, 25 bp, Artificial Sequence
  Description of Artificial Sequence Synthetic primer
ggcacataggaggagtgtggtcaca

SEQ ID NO: 5 - protein, 567 aa, Homo sapiens
MWLRAFILATLSASAAWGHPSSPPVVDTVHGKVLGKFVSLEGFAQPVAIFLGIPFAKPPLGPLRFTPPQPAEPWSFVKNATSYPPMCTQDPKAGQLLSELFTNRKENIPLKLSEDCLYLNIYTPADLTKKNRLPVMVWIHGGGLMVGAASTYDGLALAAHENVVVVTIQYRLGIWGFFSTGDEHSRGNWGHLDQVAALRWVQDNIASFGGNPGSVTIFGESAGGESVSVLVLSPLAKNLFHRAISESGVALTSVLVKKGDVKPLAEQIAITAGCKTTTSAVMVHCLRQKTEEELLETTLKMKFLSLDLQGDPRESQPLLGTVIDGMLLLKTPEELQAERNFHTVPYMVGINKQEFGWLIPMQLMSYPLSEGQLDQKTAMSLLWKSYPLVCIAKELIPEATEKYLGGTDDTVKKKDLFLDLIADVMFGVPSVIVARNHRDAGAPTYMYEFQYRPSFSSDMKPKTVIGDHGDELFSVFGAPFLKEGASEEEIRLSKMVMKFWANFARNGNPNGEGLPHWPEYNQKEGYLQIGANTQAAQKLKDKEVAFWTNLFAKKAVEKPPQTEHIEL

SEQ ID NO: 6 - DNA, 30295 bp, Homo sapiens
gcacagtccctctgaactgcacagagacctcgcaggccccgagaactgtcgcccttccacgatgtggctccgtgcctttatcctggccactctctctgcttccgcggcttggggtgagtccttctgaaatcaaatatgcggggcactttttgaaatccttgttctgggccgaggtgggcgcagatgcgtagaaaggcaaagacacaacaggtccggctctgcggcggggcacgacttccatattaggacttggacttggagcagctgagcccggccagcctggcccgcgcggagacgcagggagggtgaacatcctgagatcgccctgtactgctcgcccaaatgcaggccgacagccggcccacctcaggccgcacacacgagaaatttaggcagctcagtttgctgtcccttaattagtccccttacggctggatcacggagagcggctcaacataatattaagagcatcgactctggagccagagtgcgtgggttcgagtccccgctgggcgcttcctcgctgtgtgtccttaggcaagggactgcgctattccgtgcttgggttaccccctctctgaaacagaggtggcagtgctgcctacctcactgggctttggtgaggaggaaatgggtggctgtttctaaagcactcttggcagcgcctggtatgtagaaagcacgaaaaagggatggataaataaaggcgctgtttaaaaacgtagtgtatgaatagaaaatttgagctgtggtcaggccactatatcaggacctcttgccattaaaaagacagtttcttactcatgttctcaacaggagggggacaccagtgcgcggggcacaaggggaagcagcagagcctggggaggggacagggcggatctgaggccaaggggcttttactgtggcttccaaggatagaagaggaaaggcagggcaggcaggctgaggactggcaagttgggatacttcaggggctctggggcacaggggctgttcctaattgttttgtacctagccctggcatgattagggcaggtgggtagtggccagggcatggcaacccgatcaaagaggcggttggagtgtgggctccaggttggctggaccctgggagggacagtttctccaaggtcaacaaggcctcagatgccagaacatcagaatacagaaagcaaaacagatggtacatagaggggtgcagcgaggggcctcgaaatacccccgtgccattttatagacagagaaactgagactcacacagggaaatgtacttgcccagggtcaacccgaaaatcacgcctaggtctgtctctgggtcctgaatcctgacagcccagccccgtctgcatcctgtcttccttctggcagcctgacactgcgagagctctggaactgtggggaagggcagagaagagaggaagcctatttggaactgaatgttcctttacctctggttactcctggcaatgccatccatttccttccttaggaagaccatacaggctgcctccacttcctcagtttcctcctctgtaaaatgggcatgttgatgcctacccagcaaggcttatttgaagatggatttaaacaaactggaatgcatgcctggcacatagtaggtgcccaccatgttcgtgcttcatataacatcagcagcaactggtcttgttctcacacttgtttctgcctccccccattcacccactttttcacccttggcaatctggctgccatccacctcatcctgttgaagggaatccctgagctgagcatgaccagcccagtggtttcttctctgacttgactctcctccacctccctgaagtcctcagcaatcttagctggttcctcgccctcttgctccctgggcttcaaagacatgggaacgttctctctcccctattctgatggatctttggtcttggccgtagtggtccctgtgtttgacctttgtttttttttctcttccctctcacactggcatcttcaatgaagccatacacatccaccgtatgtaccctcacagtccaggagaagattctgccctgctccctcaccaggcttccttccagctttgaagtgcgctcatcctgtggcatcatcttgcctggatatcccactcacatctccaccgagcctgtgtaaacagggatccagcattacaagccagctctttggtcttctttcccttccatttcccccacctctggtcagggaccaagtcttttcttttcctttcttttctttccctccctccctcactcccacttgcctgcctgcctgcctgcctgcctgcctgcctgccttccttccttccttccttccttccttccttccttccttccttccttccttccttccttctttccttccttccttctttccttccttccttccatccttccatctttcctttccttctttccttcttctctctctctggaggagccaggattacaggctcatgctaccacacccagctaatttttctattttcagtagagagggggtttcaccatgttggccaggctggtcttgaactcctgacctcaaatgatctgcctgccttggcctcccaaagtgctgggattacaggtgatccactgtgcctggtctccaagtctttcaaaataccttttagacatcatcatatttctacatattttttctttttttatttaaaattttaaaataattaagatgggggggaggatttcaggtgaagaccggaaatcccatacagtgctgaaaaggtggaaatcaccaagaaaacaggccacatgtaatgccaccacccattgctatgtcgataaacatgcttccaaggaagttcatctcagtattatttgtaattagcaacaacaacaaaagaaaagaagctaaatattgacattgcagggttggctattaataagatgagacatggatgctgttgtgtaactgggctttccataatgggtttccattgtgattggatattcccctttaagaccaaatctgaattgttctgttctgctctagccccacactttgagggaggtgttgacgtcttgtctgtatccaaagcagagcagctgggaggacaagaggctggaaatcatttcctaggagggaagctcagagaaactgaggtgactggagccagataggacctgaggggtgggcagcaagatcaagtgtctgaaaggccacaggctgcagatcagatcaggccagtatagtcccatgacaggtagaaccagaactgatgcggaggctgtgggaaatgaatttcagctcagtgaggaaacattgtccgtgctgcaagaatggagcagacctgcccagggccaccagaagaatcttccttgacctgatcacatcctttatctggaacggttcctgctgcttatttgtccattaaagaaaactcaagcgcttagcctggcattctaagccctccctgacatgtgcggtctcattccaggaatattttccactccccccagactccacctggtgcatgtgggccatgcccccttccccaggcaggctctgcattctgctgctctgagcctcagcaaattcccttctctctgctgtgtaccactctctgcttctcatctcccctcattcagtcctcccctgccgcaagtcccagctcctccatgatgcccgcaatcagaaggccttgccccctcttctaattcctagactgccttatgggtacctctcccaggacatggccacttccttcccagctgtagccccaggtgtatatgtgacccttccctataatggtcagtgcttgtgagaatatgggacacccctttttcatcctctcatccagcatgcggctcagtgccaggattctaatggataaatgtttccagagacttctaagggggaagctaaatgtcttgttctttcctagtagctctccttcttgcatttatttttagctggatgtttttatgcctccaattctagtttgcccattaaaccagctcaatgggttagggaggacattgatcgtcatccctattgtacatcaagagaaactgaggcctagagggtttaggtgacttatttaaggtcactcacttagaaagcggcaaactccacctggaatctgggtccagccttttgcctctgatgcatcctgatttattctccatgtccagcagggcatccgtcctcgccacctgtggtggacaccgtgcatggcaaagtgctggggaagttcgtcagcttagaaggatttgcacagcctgtggccattttcctgggaatcccttttgccaagccgcctcttggacccctgaggtttactccaccgcagcctgcagaaccatggagctttgtgaagaatgccacctcgtaccctcctatgtaagctgcggcatgtgtccttggggatgtttacctcaaagtgatgcaggaaggagtcaaggcagtcccctgatgggctgatcctttgctctggactccttaagatctttgtagatccttaagaacattccagaactctcacagcattctggagtccattatttaacacatgtttattgagcacctgctgtgtgccaggcgtgttctgggttcttgggatccattagtgaaaaagcaaagatccctaggttcctggagcgtgcattctagcagggggagacagataagaacaatatgcaaattactccgtggattatgtgatatgatagaacaggaaaagtgccttggaaacaaagaagcagttaagcaggtgagggagatcagaaggaaggatcccatgaattctgctgtcttgaattttgacatagtcggggagaggagtgggccaggaggtgtgtgggtggcaagtgtactgaggtgtccactcggcaagtgctcaactctcaggtgtgcctattcctgaggagaacaatggaaggtgtgtccattcaccctggccaagctgggaagaaaagcccaaaggttctaattggcctcaccatcactccccagggtaggaaggaccctacagacatcagagcagaccctgctcatctcagcagccaggtgttcggaggctttccagcagcccaccagtggccgccttcttttcttcccttttttaaaaaataatgtttactgtttttattttctgagtgcaaatgtaatatatacttattacagaaaatttaggaaatacagagaaaggaaagtaagaaaattaagatcacatacaatattccaccacccagagagaacaactatcagtattttagatggtaaagtattatgatgtaatatattgtttgtattccagtttttttgcctgttgatatactttctttctttaaaagggaatgctatattgagataatttggatgcctgctttgctcatttataagtatatcatgagcataattccatgccattaaatagcacagcctggtattaaaagctgcattacattgggtattgaaccataattaatttccccaagcttttatttggggtcattaagttatatatgatgtttttaataatataacatcatgaatgtgaaacatttatgcacattcatgaatagtttaggataacttctcagaaatgacatttctgagtcaaagagtatatatttcttgtttgatttccctcccaaaatattccaactacactcctcccattgacgtatgaacatctccatttggcagactcttggcaatgcttgggattattaaaaatatttttggctattgtataggcaaaataataataacaataccagcaatattattttcctctttaatttgcatttatctgaagaccagtgagttcttttttcctttcttcacattggaatgtattattattattattattattattattattattgttattcctcttgcacttggtgatcttaggaaaatttcccaggatggaatcaagaagtcatctttgctggctctgattctgtcacttgagagctgtgtcatcccagagtctgagtgcctcagtttcctcatgtacctagtgggggcggtgatgactgccttttggtacattgaggtctgggactgttgcttataatatacttgtgtataagcaccacatggcatggatctacctggctatgtaaccttggtcaggttctctaacctgcctaggcttcagttttctcatctgtaaaatggagataatagaacctacctaatgggactagtctgaggcttaaatgagttaatatacaaaaggtacttaaaacagtgcctggaacactgggagaactaaataactattaccagtgttcatctatgttgcatccttgagatctttcatcagggcaacacaacagcaactgcctgggagaggcaggtggtagaatcttggcatgtcagggctgcaagactcatgtagaaatcactccgctcacttagacctggcggattgggacgcagagtgtaaaaggaattcacccaaggtcaccccataaatgcatgggagagacaaaactagagcacaattctcctgatgttgtcccacaaagatatcacccctgagctattgtgagaaatgtcacctcccagggaggatcagtgtattagtggtctcccctccttgatgctgggagttccaaaggctctggaaagggaagaggtgtgaagcccttctcactctgcttggtcttaggagaccttagtgagtcccagcgcccccacctgaagcccctgatagcctcctacccactacaatgtcgtgagtctgtaactatcaagtccatttccaggcttaaacctcccagtccaaggcgctgccgtagaaaacacctcgcttagatctggctgaacttcaggggttcttctttccccctccccaggtgcacccaagatcccaaggcggggcagttactctcagagctatttacaaaccgaaaggagaacattcctctcaagctttctgaagactgtctttacctcaatatttacactcctgctgacttgaccaagaaaaacaggctgccggtaagttgtgggaccccctggtcaaggcgtgtcagtgccagtaccccgcatcttctggggcagaatggaagggagtgaaggcagcttggggagggagctgcacaggccaggattgtttcagagaccccagggcctccacaggaaacactggttttccacacatcagtttaccctcgtgacataggcactctgggggagtttgttttgcatctttaatgaacatcttaattgttccaagttccctcatgctaatagaaggatgagaatgattacttttagttactagattaaaaatctgataccaagattgggtaagacatgattttcaatgttagtggtggaaagaacttagagaaaagccaacaccccaactcttccttttcaaatagagaaactgaggcccagacagggaaggggaggtcacccagtagggtagttttggctcaagtcttctgatcttcacatcagtgcttttgacatggagtagggcattgagcttgtatgtggcctttctccagcacagcaacattcatctggggtaactttcactgtccaggtacggatgttgggggcggagcctggacaatcccagaagctcttccttttgcctttggccaaatagctgggatttctttttgtacctaatttccctccctaattgtcttggcaaaggctgatgacccagtgagtcagaatttcccaaataaatgaagggcaggacatggggcagcccgccagctgcaggttggagggtggggagggtctgctggcaaaggaggcacccagggtgaaaaaacaaggggtgttgtgtctagccagtctcccaggggcaccgtggcatggtgcctaggagtgggggagcaggtctgcgaacgtggaactagaagttggtcctacatgtgctttgtgagacacaggaagtgagagaaaggtttactcattcattcattcattcattcattcattcattcattcttctattgtgtactcagtgccccatatacacagttagacctggggcttcagggaaaggaggaacatgaatccactccacacagcccctgccttcaaggatcttgccttctgggctgtagagacatatcctcccttcagcactttattgcaggccagccctcaggctcagtcttcacacaaagatgaatgcacactcaatccctccccaggaggcaggcagggattagagttgtgggagaaacaagggctctgggaacctggagtcaggagaacctccaggaatggccacaggctctagaactacacggtctgggtttaaatcctggttctgcttttgactagctgagagacttgggagtttcttaacttctctaagcctctgtttcctcaaaatgggaatcatcacatatgatcagtcaagggtgaccactaagcgtgatctcagtccctggctaaaaaggcagaggcaggctcatacccctgccctaacagtggactcactgcactgtggccggcgcctgcagtgtgctgggcagctcaacatgtggactctggaggcagactgcctgggttaaagtcctgctgctaccacttttgagttgtaggaccttgggcaggttaagtgcctccattttcccacctggataatgggacattattagtacctacccaatgtatagtttattttactgtgcttagaacagcacttgggacatagtttatgttacgagttcttttatatttcatgaataatattattacaatatcattattttagccactctcacttcttccccactatgtggacttttaaaatgcaagaatcatgcatgaggaattgtatccactctcatcctccagtattagcagagtccctggcacatgctaagttctcgacagtgttagtccattgactgaaggcatcctgaagtgggtgggttttaggctgggttctgcagggtggataggatttggggatgcagaggcataaggaggcatttgaggaagagacaccatcacaggcacgtgtgcttctcgagacaatcattcatgcacagagaagtttacttcaccaagagatagttaactgagatgaagctgagatacaagggctgccccatgggtacgctgagtgcatgaatagtctaggcttgagggtgatgggagtgtcctcccgaagaggacatccttagcctttgctatgcccatgatgatgttctcagcatcaagagcctttcggggaggaggcactggaggctgggtagaaaggaagggacagccagggtatgtgcagtagggtggtggcttgggtcccagttgggagaattatgcaggagagagattgccttttgcaaagttgctgggagctggtgaactttggatagcatcctccttcagttgtaaccaacaggtaggtcaccaaacaagacatcttctgagtgctccctggctctgtgtcctgtaactggtatgttgcttcctctctgcccaggtgatggtgtggatccacggaggggggctgatggtgggtgcggcatcaacctatgatgggctggcccttgctgcccatgaaaacgtggtggtggtgaccattcaatatcgcctgggcatctggggattcttcaggtaagaaatcggactctcctcactgcactttggcccccagaacgaggatgctaggacccagctctggtcatgccagccctcaggggagcttagctaggttccacagtaaggcatccaagccccttcgtaattggacactacctaccctctcactacccagccactcatccacttgcctctgagcttttgcatgtgctgttccctctgcctggaatgcttattctatccgaagaacacctcttcatcctgcaagacccagcccccagttacctccactgaaagactcatctcctcttcacctatgtttctccagcacattgcttttctctgtcatgacacttagcagtcagcccagcaggtgaaggttgacacatctgacttctcatggaaaggagggaaggggcagagtcacagagggagctcagggcatgttgtgggtgaaggaatgggggctgcagatggtggggtgtgggagcatctgaccccttcctgtgggagtgttgggacccactcagctcattttctgcttgaaatttggcaagtgtgggaagcaaggatggagtcttcccatcaggtctccatggggacaggagtcagatctccttcaaggcgcaactgctatgaaccttttctaaggttctcctccatccatgccagtcaggtttcttcctgaacacgccactcagttggtctcattgcggtcatcaatgacctccccctgctctccacccaacctctcaggacctcccagcagcagcagatggccactcagcctctgtgacatgcttccttcccagcccctttcctggtcctccctcctccctgtccacaggcctctcttgtctccctctccctccttactaattcccagtgatcccagggagcagccagagcccctgtttccagcccacctcacctttgaagcccacaggcatcactcactccctcctccccacccctggctgtctaaagggtgccccactcttcccagggacaagttgaatccttgactcccactgcccctccccaacagagacccccttctcctcccaccacgcctccactcagcttctgctgccacatcctcccttgggctcaggagaaggccttgtggacagcccggactgtctcttttctcacaaccctcttggctgcgccttcagaatccactcagagtcccctactttgcacctcttccctggctcctagcccagccaaagctctctgtctgtcaccttgattatgatctttaacactttcagatccctgccacttttatataaagctgagttgcacagtagccagagtgatcacttaacacatgcattgaatcatgcgatgcctctgcacaaaaccctgcaggggcttcccatgctccttaccatgacagccgaagttcttgccaggccccagggccctctctagctgcacctcctgactcacactttctttcactgggccccaggccctttgcacttgctttttcttctgcctggaattcctttctttggatatttacaaggttcatgtgctcattgtgcttgttttatgaaggtctttgcctccaaaacctcaaatgtcactgcctccaaaaagccttccctgattgggatctgaaatggcaccccctgtcccactcctgtttttttccttactccattgtatttacctcagtgcactttgactacctgacatcattatattctctctatgtatctgtttatctgcttgtgatctgtagtgggaattcagtgtcatcacaggttttgcctgttttgctctcagatgtgctctagctcataagacagcgtcaggcatacagtagatgctcagtaaatagttgccagttgtgtgaatgtagaaccatacatcaccacaatgctgtactaatgaaagcagggtctcagagatgctgaagcccagccttattcttaagggttcactgagaacccctagccccatctgtggtcctgaaggtcctgcatgacatctctgctccccaccctcaacctgttctcttcctcacagcacaggggatgaacacagccgggggaactggggtcacctggaccaggtggctgccctgcgctgggtccaggacaacattgccagctttggagggaacccaggctctgtgaccatctttggagagtcagcgggaggagaaagtgtctctgttcttgtgagttttcctgtcaccaggcccaaccccacgcttgatgtgatgctgatgagacctcttggacacctgtcaatccagctatggatacctctgattacaatacctgaattcaggactggccctactcacagcccagcatcagggggtagaaagctaaagaccagctctccttggcccccaggaagctcagagctcagcttagtgtcctggggccatatgaatctccagttgcagcctgcaatggtggcattctcctcttccagcttggttgagctctctctctctctctctctttctctctctccttcacactcattttaacataaggactcacatcccttctcttgggaagtaggaataggcataaattatgagtaagggcaggcagagagaagaggtggacagaggtcattgtttggctaaaccagacctacatgtggaggggacatggacactgagcaggctgggaattcctctggggtggtctgatggcttgtccatgcccaagaaggaggcgacagtctttgtgactgtggggtccagtgggctaggggaggcaggcagaggaaggagggatggagccgcggatagggagggatggggcaggggttgtgggtcatagacacaaccttgggtgtgaggggtcctgctgggattggggattgggttcaggagacactgggggatctgggatgaaaacccagatgagaggtgctgggagctgtaggaagacttccacctccttgaggtgggcagagggtcagcccactactggattcctcagtcccgtgttggttttatagtggagtagatctagcctggaatagcgagtgagtcactgaccccactcctgagcatgaactctcctcccctccactctgctgtcaggttttgtctccattggccaagaacctcttccaccgggccatttctgagagtggcgtggccctcacttctgttctggtgaagaaaggtgatgtcaagcccttggctgaggtaggtctccggctggtacgtctccggctggacacccccacctccttggctctatgctcctgaatcctcagggatctctcttgtggtcggttgtagctaatgttctcctagaatcactgaggcaccaatggctgagcaggaagggcgaggagacaccttgatcagcgtcccagtttcacagccaggcaaaccgacacagggcttggaagggatttgccaagggcagcaggtgatcagggcagagctgggactccagctcatggccctagcagccagtacagtgccctatctgtgaccacactcctcctatgtgccagggcctggtgccatgttgggcagtgatggtgtcttgtgtctctctgggtctgcctaatggctggtaagtggaacaaccaggatttgaaagcagacaaggaaattcaggattccatgctctctatcccagctccacctcacatttcttgacacattcaccttgagatgattatgtccatttcaatggagataaggtagcagagatgagagattacataattgacccatgttacaattgagattagtaacagaggcaacgctccgtgggacctggaacccacacccacgggtctacagtatcttctgctgctccctgccactagtacaagttgggcttgggatagaatgccactttcctctttgatggagggaagggatgtcgctcttgaactctgttgttgcctgtgatctttgcagcaaattgctatcactgctgggtgcaaaaccaccacctctgctgtcatggttcactgcctgcgacagaagacggaagaggagctcttggagacgacattgaaaatggtaggttgcctgttcccgtagcccaaaccctgtaaacttggtcccagacttcttcatttcagctgtcctcttgcccagggacagtttcctgggacaatttctcaactctcagtatctgaatggggaatctgatttgtccttttttattgtaaaatgccacaaatgtaaaaaaagaaagtcaaaaaataacatgataaaaaattgtgtaacaatccccagtcctgaccaaaatttaatattttgccattcttgttttcagatgtatttttaagaaattaaatgttacatattcccaggggacaccatcatcctgaatttgggggatggagatattaagctcaaagattttcagaaagatgtcacaatttatcttggttgacttagaaactgtctgtattagacctagtggtggtccagttttctagattttctgagactgactcagttgtcatcctaggatagtcatccatccacccattcgttaatccgtctatctatgattgtcttatccatctatctgttcactaattcatccaattcactcatatctttttatcaatctaatgtcaacctattcataactttgtatttatctattttcaatccattcaccattcatggatcatccagccaccttatatctcaactccatcacccattcctccaaaatcaacaatccaattatcgcctgtctgctagttttcacccatctattcatgtatccattcaattcaactgtactccatgtattgaccaactccatccatccctccattgatccatccatccatccatgctaaatatgtaggggtgggtgttagaggtagcaaaacagacatgaagtggacatagtccctgctctcaaggaactatccaaagagaaatacattcatatacttcgcaggttgagtatggtggcagcacagaggggaagcattcattgtagtaatcagggatgcttcacagagaaggcggaggagccagatttgagaccagacagtggaattcaggagttcatgcccttaatcctgactccaccttatctttcctgagaaattcagctttcagatcattgtgcccattttaacagaggtaacaaagacagataaattttgtaattgccccatgtcacagttcaaattagtcacagaggcaacactgtaggacccagaaccgacacctgcgggtctagagtatcttctgccttccctgctccctgccatcctcacatattaggacttggggacctttaggattgttggatggccatggcagtctctcatcacagggaagaccaggaggacaaacacccagatgacacagcacccagggccattgggaactattccctttgagggggaagagtgtgaattccaagctcaggagtagcctggactctctccttggacctgaggctactcctggatcccagggctggcctctaccactggactctgcttgtgtttccatgagttgagtccaatgtggtattggtgcttaggtcttggctcaggctctaagtgaccaaagtgttcaaggaatgaaaacacaactgttttctaacgcccggaaggaggcaaatattccagcaattctgcatgtggcatcagaggacccagcttaaaagggaagagttgagtctttgggaaaacccatccctaagatcctagaacattcttttgagtttttctgagatcttgtggaagcacctccatgattacgcgctgcctccagtacacacacatgcagacacacagacaaacacacacacacacagacacacacacagagaaacacatacagacacacacacacagagacacacacagacacccacacacaaagagccacacacagacacactcatacacacacacacacacactcctggctagtgggacttcaatccttaatggagggacctgcactggttacatcctgagcacaggtcagatctgggaactacagtgcaacactacagtcactgcaatcttggtgaacacacaccaaagagaagcatggggtaggatgagactcagtgagcttgcgtgagtcagggctttctaatgggagatgaggaaactctcccaagtaccttgaacagaaaaagaaatgtgttgcaatagcatcgtcgtgtaaactgaaaatctcaggagttgatggcttcaggcatggctgggtccagatgatcataggatattattgagaatctaccattctctgtccttcagctttgcacatctctgtgttggcttcactcttaggcagatatatctcctgggaaggttaaagacaacagcactctagacttatgtcctatctgcttaaaaaccctctttctaaacaattcctgcaaaagttttggggtaaactctattggattgacatgagtgtatgcccatccctgaactaatattcatgttcagggagatggagcaccgcaggtcacatatagatatacgaattcacggagtgatgcgggaagaacctgacacctctgttgccccactcacccagctcagtgttctcctggtaagcctctcacccacatcctctgcctttgtcttcacagaaattcttatctctggacttacagggagaccccagagaggtaaggaccttttgtttctggattacgggttttgagtcttagcacctttaagctccaattaactgtgagtgaaagaatcatctcttgggtaattatagtaactcctgcgtgtttgtttctgaggcccagagaggggtagtgactcacctgggtaacacagccaggaagactagtggctggcctggaacccattttcctgactcccagtccagtgctcccaggcatcacctctgtgtgccctgggctctgcccactccccttcttttttacattttctgctccccaaaatggcactgtaggaggaggctgaaacagaaccttccacaatcaggaggcagagataacagcgatgattagccaaggagaggggaagcctaaatctcagtccaaggacactcgtctcctcccagcacacaggaaactccaacagtattctcccaatctcctcatccccactcccacccccacctcccagtgggttgacagtttctggtgacatcacctctgacgaatcttacaatcctgtcctctctgctgcctccctggagatcacagcactctcctaaatggtcatgggcggagtacatgagattcattcagcaaagacttaataaacacttcctatgtgccaggacttgtttaggagctggggatataacagtgaaaaaagaaagactccctaccctcttgggaccttccattacagtgccaggacagtgcaataaaaaagcaaatcaagtatgtattccgtttgatggtgtgcagcattaggggatagagtttgtgggtggcttcaatttaagtagcaggatcagggaatggttcaatgagaaggtggcatttgagccaagctctggaggaggcaggaagccagctgtcaggaaacctggagaagcctattccaggcagagggaacagtcactgcaaagaccctgacagaaggggccagtctggctggagaggaccgagtgtggggacagactggcttgaggctaaagaggtaatgggcagtgggagagagatcaattaaagtacttggatttggattcagagcaagatgggaagcctttggaggttttgaacagaggagtcacatgatcttagatctcacaaaggtctctgtctctggtgttcaaatagactgtaggaaaggggcaaagtggatgcagttgaccagctgggcagccactgcattgccataatccaggcaagggctcctggctgcttagacagggctgtggcagtggggatgggaggagtagttggagtctggatatctttgaaggaatagctgacaggatttgctgatggacaggatgcaaggggtaagaaacggggaggagtggaggacgccctgagttttctgacacaacaaggttgtgcccacacagggcagccacttgcaaactctaagtggagaacaatcacttgtcattgttgctgatggcagtcttccagcatggctgtcacccaactgaggtgctcccgtctcttgcctcaacttttagcctcttctacttcaccttctctccccatttccatgtctcatggtgcagccagactggccttccttaacagaattagagcacgtcacccccacttcttaaaagcctgaccttccttatgagacaaatccccttccctccatggagttagtccttcaaaacacacctgcgtgcacactgtgtgccagactgtgcagaaggtaggctctgcacctgtccctccctctagaccctgccccacagcatttctccaagccaagaactcttggttgtttctggaatttaccctgcacagtgacacctatgtccttttgctcatgcggttcccactgtttagaaattctcactcctttcctcacctggcaataccattcactgcccagagcccaccgcaggcaggcggcgtctcttccacgtagtctgccctgcatcttccttccagaggggctgctcctcttgctggcctcccacagcagccctgcctgaactgcacagcctcttaaggaggctggacttgcaccccaatcctgttcttcattgtcagccatattggattaggatctgatcttgtccagctaggtctggtccacttttcagaacatctcccccaggcagtccgaactagctcgtcattcattggcttttgtagagcagaccaaaggtcccagaagccatagggtctcaaaggctaggaattgtccagttccatctgatatccagaagggaaatacagcccatggggcgtggaactgggaagattccacagaggactggtgtctatgagagggacccacagcaaccttgctaacaagtaatacctaacagttattcagcctttccagacaccaggtgctgtgctaagtattttacatgtgttcagccatttaatcctcacaataactcacctcttagatgagaaaactgaggcccaaacaggtgaaactcccagccagtaagtagcagagccaggcttcagatgcagttaatctggtttcaaagtccatgctctatcttagcaacatccacgctctatcgtagcaactacaccattgtgactaaatatgaaatataactgtacccagaaccagctgccctgatggcaacacatgagttgggctctctctaatctgtgaccatataaaaattattcatcaaaggtgaaacctaaaattaagacatggatcaatatactgtgagttaatcactgattcttttactcatgattttctctctagtagggaatcatgtcctagtctaggctccttgagtgaccagggttcggtacctcctcaaagccacccgtggatcaataacagctcttgtttaaatacagatagatagcataatctcttcctcattaatcatggattctgtgtttataaattcacctgcttgctaaaatgtccttataacccccaaatcaatactggtggcactttcatggtcacacacaggcaggtacagagcaggggaagctggatttgcatgatgggcatgttccatctgagatccggcgaggcaacctctctcttcttgtttcatctctgatgtttacaagtgtcctcttcgcggtctatttagtgccgtgtgttctgcatttttgtgctttttgtagtgatgtcactgtttagagcgaacccaagagtagcgctgctgtctggcgttcctgagctaaacaggctgtgctgtgccttacggggaaggtgcctgtgtgagacaagcttggatgaggcaggagctgcagtgctgctggccgtgcatttggtgttgaaaaatccacataatagcacattcagaaaaaggagaaggaaattggccgattcatacatgaggctgtgatagaaagggctaaagtaacacctattgtgtgggatggagccgtggaagtctctttcaacagaaacccacacataaaagaaggttgatgaaaatgttgtggccagaggtttgcaaaaacctaacccagtatttcccccttgagtgatgaccctgtattctctaattcagtactcacgggggctttatcagaagcaactgccaggactaccgaaaatcaattgtggacacacagaggaaaatgcatgcacttcaaattgaaatatatagacatgtaattttacatagataatacatgtatttcaatatgagtgaagatttcttcctctcctcatcacctgagcagtgtcagcctctgcctgaagttctgcccctgagtcatggagacctacccccctaagttaggatcctgagcatgtgcagccatggcgcatggccatgccggtctatggtactggtctcaccctcagacaggcagagttggggacatgggtgtgactagggagggaatcacaggtgctgcactgctctctccccagagtcaaccccttctgggcactgtgattgatgggatgctgctgctgaaaacacctgaagagcttcaagctgaaaggaatttccacactgtcccctacatggtcggaattaacaagcaggagtttggctggttgattccaatggtgagaaggcacatgtctgttggagggactcaaccacccccatcagccctcctgtctctggtcccaggaatgcttccctctccgagctgcactctgagtcctgggcagctgttcccttcagcaggagttaacatttccacggaaatcttctccaggagggcacagttttcaccggggtatcagggccctgggattccctctccccagactctcttattgtcttcctattgaagaatcctgaagtgtctgtgctgggagggccagtagagaaaataactctgcagatgggaaaacggagggggcctagaagggggaaggagtggaggagtccggagaagcagtcgggggcttcctggcttcccacctcgggctgggtgtgcgtaattcccctgaaaatcactcatgctcttcatcacctcattgaaaatatccaaagaaagtggcttcttcttgagttaccattgacttctagggaacagcagacatcagcgtctactaagagagtggatggtcagaggaagatgaggatggaaaagctacctaatcgggtgctatgctcaatacctggtaacaaaataatctatacagcaaactccagagacccaaattacccatgatcaaacctgcacagttacccccaaacctaaaataaaagttggaaacaaataaatggaaaaagtaattgagttctaaaagtaaagaagtgttctccttccagttgaagataaacaaaactgaccctggttaaagcagtaggacagatttattcagtaatatgattgcaataggggaaatagtccagcgtggagtggactcacatccctgaaacaaaagcctgatgacgtttcataggccaggtgtgccaagggaaacactgtggtctatggggagaggcttggttcatgtgactagaccacctggatgtcttcatcctggctcacctggggcagaaacaaactcctcttatctttaggacaggaagccatgcattagaccgcaggaagaacccactgaagtcaggctctactctttcgacagggactgggaagatcaggagtgagctccctgaatgtttgcattgcaaagagaaggctctcaggtcctcaaggaaatggggttgggtggtagattcacatcccaaagggcagagaaagtgtttatcattgcaggcttctaaagtaactgctctagttgggttcggggacctggctgcttgtctccaggttttggctggagcagagtaaattgtcctggcagggttgagccttcccaggcagttatgttaaggatactggggtcattctagggacacagccttaagctgctagaagcaatgctagagattggtcgagtccctgagtgcagaggtttgggaagagttgttatgtgcgtagagatgtgcagttcgcccctcagaaggatttcagaggttataccaaagaccaaagccacgaagacaggatttaaggatttcagtgtcatctgtgacaggtgggggtactagggaggtctacatcctcttccccagagccctctattccacactggcctggcagacaccttgcatctccatgtcactgaccactgtagggatcccagccacagacacacacacacacacgcgcgcacacacacacactgcaaaacactgccacagcttctctgaaattttgttttttgttttttttttttgtaaactttgcaatgcttgagtttctggatataagcccatttgattcattgatttcacccatttcagtaaagactcatgcccttaaaagcccccataattagaggactttcagactgcattggtttggttggttggtcagtttgtttcttatcattaattcccaatgattcataaatgctgaactttttttttttttttagcagttgatgagctatccactctccgaagggcaactggaccagaagacagccatgtcactcctgtggaagtcctatccccttgttgtaagagtctaggaatcatgggaattggctgagacccagagagggacaaggacttgcccaaatcatagagcaattaaatggcagaatgaagactggggcctcagggtttcccccatctttccacctttcccacttcattttccaccctagcggggagttgcacagggcttatggggttcaccattgaggcaggactttctggtgggctggagaagctgcatcgctcacccggggctggtggtcactttttgatctatttcagtgcattgctaaggaactgattccagaagccactgagaaatacttaggaggaacagacgacactgtcaaaaagaaagacctgttcctggacttgatagcagatgtgatgtttggtgtcccatctgtgattgtggcccggaaccacagaggtgagtcccagaggtcgaacgggagggacacaaaccccacgagcttctgtatctgacccacctacctcccagcatagacagacatggaaacagctgagggtcaggcctcaaaggctgattccatatggcatgggatgaggtgctgtcttattttggtttatgccagcagaagactgtgagaaaaaaaaatccagaggcaagtggtttttttaaggtgatgatcccagataacagcagtagggaggtggggaagtgagagaggaaagagatggaatccaatccaagatgcgttgtcaagggagtatccactgtggacaactggagcggggaaactcaaggaaacaccaagaatacagggctcaggggcatcccatctgagtggcaagggagccgggtatttattcaccagcttctacttgtcattggttgaaggctcttgcaaggaattgtttattccctgtgacttcgacctgctgcacacaagggcagagtagtctcttgtgaccagacaaaggcctcaggcctggagctgcagatgctggaggtggaagtcaggctggcatgcccaggaagaggggatataggtgagaccctggcagcatctgctgcaagggctccatgcctggagttattgtgggacatagggctgctgtaggacaaatattgaaagatgagtcgtggaagggttttaaggtctgagccagctgcaaacaagtttgttaagtattggagctggataaaaagtgggagccctgcatgggatccagaggattgtccaggaccaagaaggccagagagaggagccagggcgggtgtgggagaggtggtctgtagaatggcagtgtctgagggtggtttttatgcatctcagtgcgtgcagtagcatgcaagcacagggcaagggcagggtctatgcaggacctgctaccactgacccaaggctgtgtgggtggggcatgacgttagggatgtgtgtgctcacctagagaggagtgtctgttcctgcgtgccagctagaggagattcacaggattccttttcttccagctctcattcgcctagtctgcaagctaggaaatgtcctctctctaaccatgctggaagggtttttaaatttagaacttcataagcatgataaaaactagctaacgcttaactagcaggtgccctgacacacctttgcacaggaaggggcaggtgctcataaccctcatttcttcacagggtcctgataaaaacctttaaccaagaccactggagttgaatctgttctttcttaaacttgcccttgccctatgctctgcgtctgaactatatatagagttcccatctccattttctgatggtttgagcaatactgaacttatgtttttcatctggtttcccatgactttaagttcagggcttttttttttttttttttttttgccaggactacacagattttatctactttcacttattgttgtctcactctttttgataaaataacatttctgaatataaaattaatatgttaaattatagaaactttcaaaaatatggaaacacaaaaagaagaaactatgtatcactcatttccactacctggaggcaagtgctataaattttttgtgttttcgtctaaagattttccatgtcctatatactttaaaaatcaattttatattttacttttgttcatagaacactatgttgtgaggattttctcaatgttgatacaaactcatcaaagcattccttattccatgaaatggctgtgttaaatagactttaatattcatatcctattagatgtttagcctggctactcacattctgatgctacaataacattgagctcagcctaatgttcataagtctctaaacacatgtctaataattttcttaggattatttttagaagtaaataactaggttggacaatgcaaatgttttagaactttgggcaaatattgtaaaattgcattccactgcataagaatgtggatttcttaattctctttctcatttgggatgctaagattaaaaattagcataacacttccagtttcataagccttccccaccaacaagaagcacatcagcctgttttaatctgcatgtcctccatgactagccctctcattgtctctgcttctcttattatgtctaattgtcattacccaaatctgtgcgctactttcctctgaagacttgtgttgtgattgtaagaattgtataaatatcaatggctttagccaaagagttttacaaagtacgttttgggcagcatgctgcctataataagttctatcattcacaaaagctctttcttctatggttggctctgagtttctttggaaaatgttcgaggggcaaaggcaaggtcaaaccctccttttagcaagttttgtttgaccttgaagcagccatttaacaggtggattacagagccacagaaggatggcatcttcccaggtgcgcttcctgctgagccgagggtgcagtgcaggaggctgacaaagatgagtgaatagattattcttcattctcacattgaaatgacatgtgcgggtggggcatgaggttagggatgcgtgagctcccattgttaaatccttttgtgaacgaatttttaatgatgttaagaaatgatggctggaggcggtggctcttgccaacactctcagcattttgggaggctgaggtgggaggattatgagagcctaggatttcaagatcagcctgggcagcatagtgagaccctgtctctacaaaacaatttaaaagggaagtgattacagtgtactacaaaataccatatataatatcatccccagttaaatacataaaagagagatatgctaaaataaaaaagaagcattctctgagtggtagagttatgagtgatactttaaatttgattgttaaaattgatttttaaattatataggatatggaaaaattttagaaggaaatacaaaaaatgcatggcccttgcctcaggtagtggaattatgagtgatatctagttttgctttatgcgtttccatatttttgtttcttctaattattcctaattgtttttattttccaaatttcttactgtacccaaaggggaggacattgtaaattttctccaccagcctggcattgccctgactctttccgtcttagtgtcacttctcagtgggtatcatgaccagtgcaaagtgctgaggcacagggccaggcggcccacgaggtagccctgaaggggcagcgatctgtggctccaatgagccaatcattgggaaactggcatcttcctggggctgggacaggagagctttgttggtgggagaggagggcagagccgaaggagagagggcaggggagaggcaaggtcacccttgctcagcgccttcttttctctgccagggctttactgccttaacaccctaacactcccaggccaggcaggcagcaagacacatggggacagtgagctataaagcagtgagtgccgcaggttcctgagacacaagaggcagattgttccctctctgcatatgtttgtatttaaatagcctcatgttcagacaagcaacttggatttgcttcagtcttctcaggatgggtgagaaaaacactggaaatgctcaaaacgaggcagaacagaagacacctgtcatggtggagatggcatccaagtgaatgaacgcgagtctgttatcaatgaattatatccggtgtaaccagatgtagagagattgtcttgtgtgagaatgcaccagcaggcaaaggggtttacgaggcagagtcagggtccctgcaccggcagctgttttaaatgaattgacccagggtttccacattcattcattaaacatttgttcattgaacaaatatttggctctaattccgcttccacctcagacagctatgcagcccggagcacatcactgagctttcctgagactctgtttctctactgtgctgacagccagaggtttgctgcagagctggagaccttggtagcctccaggtgtgtgtctggcaacatgaactgtgcatgaatccttgtcttctcagatgctggagcacccacctacatgtatgagtttcagtaccgtccaagcttctcatcagacatgaaacccaagacggtgataggagaccacggggatgagctcttctccgtctttggggccccatttttaaaaggtaatgctccttcctgtctgtgagctaggagttagagacttgggttccagtcttggtgtggtggccttcagcaattttgtcctctcctggtcttagtttcctgattctgtataacagggttgaatgtcactgggttgtccgccggcttcctcccactctaacacatgtgaggagggatgtgtattcatgtgccctctgcacagacgctcagaatcctgctgtgtgacatcaagacaggtggtggtgggggtctcctgcaggattcactatgacagggttgaatgtcactgggttgttccccggcttcctcccgctctaacacacctgagggaggatgtgtatcatgtaccctctgccacagacactcagaatcctgggctgtgtcatcaacaagataggcggtgggacacatgcggggccctgctctaggttgagaagccaccaatcaatttccctctcccaggagcgctgagacagtgagtgagggtgagattagaattggtgttaaccctaagtcaggtctaatgtgtgcctgtttgtctttctgtctgtctctaacaaaaactcataaacctttacatttagtacatttaataattgttgatttagaattcacaggatgtatgaatttggaacaggagaagagaatattttttatatggtttatagtcagcaaggtggccatcccacaggctgggaaggtgcctctggccaagcccagaaacgggctcattggaggaggggttgggtaggagctttatgtgaacggattggctaaacatacatgttcaacaggttacagggggagcaatggatattcatgaagacagtcctgacacatgtgtattaaacaaacatgtatgtaacatggcccatgttcacctggtggtggagacctaatatttaaatgtattacaattagggcctgtaagtccaaaggtcttctcaggacacgaagctcagcaagtgaggagcttctgtacaccggccaggtccagtccatggctggtgatgttcttatctggagaaagttactgaaatcagtctcttatgcaatcaaagcagtagttaagtctggcaagtcagggttctgtttgtctgcgtatcccagctgcagccgttgttattgttttccttttgcttagctccaggccagtgctggtttagctgctagagaaagagaagcacctcgtggcagtgagaacaaagtggattcttttttttttttattattatactttaagttttagggtacatgtgcacattgtgcaggttagttacatatgtatacatgtgccgtgctggtgcgctgcacccactaactcgtcatctagcattaggtatatctcccagtgctatccctcccccctccccccaccccaccacagtccccagagtgtgatattccccttcctgtgtccatgtgatctcattgttcaattcccacctatgagtgagaatatgcggtgtttggttttttgttcttgcgatagtttactgagaatgatggtttccaatttcatccatgtccctacaaaggacatgaactcatcattttttatggctgcatagtattccacggtgtatatgtgccacattttcttaatccagtctatcattgttggacatttgggttggttccaagtctttgctattgtgaataatgccgcaataaacatacgtgtgcatgtgtctttatagcagcatgatttatagtcatttgggtatatacccagtaatgggatggctgggtcaaatggtatttctagttctagatccctgaggaatcgccacactgacttccacaatggttgaactagtttacagtcccaccaacagtgtaaaagtgttcctatttctccacatcctctccagcacctgttgtttcctgactttttagagatgcaggcctgaacccttgcccgacgtggccttaggtctcgtttttaatttggtgtcttattgccacaaagagtgtgttctgtcagaatgatgaccttcattttattgctgatgctggtccggtggtgtctaaatcacaaaagggagggagtattatgaggcgtgtctgacctcctgtccgttcccaggcaggaacagagttgaaggtttttcgaggctccccttagcccagagagggtctgttcagtcagttgggggtgtcaggattttatttttagtttacattatacaatagtttaaaaaatcctttcacggtgtcttctaagtcttaactctcagcagttacagatcagatgggactcaccccatggctgctcagaacgcaccagcgcctctgggcatctcactgtgcatgcttaggcgccttgcggctctgttgtttttcagaatgtgaattgtgggtgtgtgctggggagggagaaaagacccaagagagagcaaccctggaaagtggggttgctgtagaggaaaccctggggtggggcatttcctgtgaccctgagcggggaggaggcaggcgagggtcacaggagacaaaggcagccagccaatggaatgacgcatgcccattgctgagccctaggtcagatatgctgaaaggtaaagtacacacaaatccaccacgatcttctaaaagtgcagacctcagctcagtgggtgtggggcatgtgccaagactatgtttccagcagactcgcatgtgatgttgacgctgcagtccacggaccacactttgagtaacaaaggctttttttcttcttccccacagagggtgcctcagaagaggagatcagacttagcaagatggtgatgaaattctgggccaactttgctcgcaatgggtgaggctggtggcaaagacagagcacggctggtgagggtggggggcggggcatgcctattgggaaggggcagcttctaaggttctagtgatcaaacttctgaccctgtgaccatagcactctgataatgagagctctctgcaaacggagaggccgcccctggagatagtgagctctccatctctggaggtatacaagcctcttgacagagataacttgggcatcctcacacatctctgaagattgttgggaacacacagcagctttggggcaattctaattgattctgtttccagaaaccccaatggggaagggctgccccactggccagagtacaaccagaaggaagggtatctgcagattggtgccaacacccaggcggcccagaagctgaaggacaaagaagtagctttctggaccaacctctttgccaagaaggcagtggagaagccaccccagacagaacacatagagctgtgaatgaagatccagccggccttgggagcctggaggagcaaagactggggtcttttgcgaaagggattgcaggttcagaaggcatcttaccatggctggggaattgtctggtggtggggggcaggggacagaggccatgaaggagcaagttttgtatttgtgacctcagctttgggaataaaggatcttttgaaggcc

SEQ ID NO: 7 - DNA, 30275 bp, Homo sapiens
acagtccctctgagctgcacggagacctcgcaggcccccggaactgtcgcccttccaggatgtggctccctgctcttgtcctggccactctcgctgcttccgcggcttggggtgagtccttctgaagtcaaatatgcggggcactttttgaaatccttgttctgggccgaactgggcgcagatgcgtagaaaggcaaagacacaaaaggtccggctccgtggcggggcgcgacctccgtacttggaattggacttggagcagctgagcccagccagcctggcccgcgcggagacacagggagggtgaacatgctgagaccgccctgcactgcttgccccaagaaggccggcagctggcccacctcactccgcacacaagagaaatttaggcagctcagtttattgtcccataattcgtcccgttatggctcgataacggatagcggctcaggagaatattaagagcatcgactctggagccagagtgcctgggttcgagtccccgctgggcgcttcctcgctgtgtgtacttaggcaagagactgagctattctgtgcttgggttaccccctctctgaaacagaggtggcagtgctacctacctcactgggctttggtgaggaggaaatgggtggttgtttctaaagcactcttggaagcgcctggtatgtagaaagcatgaaaaaggggtggataaatttaaaggcgctgtttaaaaacgtagtgtatgaatagaaaatttgagccgtggtcaggccactatatcaggacctcttgccattaaaaagacagtttcttactcatgttctcaacaggagggggacaccagtgcgcggggcacaaggggaagcagcagagcctggggaggggacagggcggatctgaggccaaggggcttttactgtggcttccaaggatagaagaggaaaggcagggcaggcaggctgaggactggcaagttgggatacttcaggggctctggggcacaggggctgttcctaattgttttgtacctagccctggcatgattagggcaggtgggtagtggccagggcatggcaacccgatcaaagaggcagttggggtgtgggctctggggtggctggacgctgggagggacagtttctccaaggtcaacaaggcctcagatgccagagtatcagaatacagaaaacaaaacacatggtacatagaggggtgcagcgaggggcctcgaaatacccccatgccattttatagacagagaaactgagactcacacagggaaatgtacttgcccagggtcaacccgaaaatcatgcctaggtctgtctctgggtcctgaatcctgacagcccagccccgtctgcatcctgtcttccttctggcagcctgacactgcgagagctctggaactgtggggaagggcagagaagagaggaagcctatttggaactgaatgttcctttacctctggttactcctggcaatgccatccatttccttccttaggaagaccatacaggctgcctccacttcctcagtttcctcctctgtaaaatgggcatgttgatgcctacccagcaaggcttatttgaagatggatttaaacaaactggaatgcatgcctggcacatagtaggtgcccaccatgttcgtgcttcatataacatcagcagcaactggtcttgttctcacacttgtttctgcctccccccattcacccactttttcacccttggcaatctggctgccatccacctcatcctgttgaagggaatccctgagctgagcatgaccagcccagtggtttcttctctgacttgactctcctccacctccctgaagtcctcagcaatcttagctggttcctcgccctcttgctccctgggcttcaaagacatgggaacgttctctctcccctattctgatggatctttggtcttggccgtagtggtccctgtgtttgacctttgtttttttttctcttccctctcacactggcatcttcaatgaagccatacacatccaccgtatgtaccctcacagtccaggagaagattctgccctgctccctcaccaggcttccttccagctttgaagtgcgctcatcctgtggcatcatcttgcctggatatcccactcacatctccaccgagcctgtgtaaacagggatccagcattacaagccagctctttggtcttctttcccttccatttcccccacctctggtcagggaccaagtcttttcttttcctttcttttctttccctccctccctcactcccacttgcctgcctgcctgcctgcctgcctgcctgcctgccttccttccttccttccttccttccttccttccttccttccttccttctttccttccttccttctttccttccttccttccatccttccatctttcctttccttctttccttcttctctctctctggaggagccaggattacaggctcatgctaccacacccagctaatttttctattttcagtagagagggggtttcaccatgttggccaggctggtcttgaactcctgacctcaaatgatctgcctgccttggcctcccaaagtgctgggattacaggtgatccactgtgcctggtctccaagtctttcaaaataccttttagacatcatcatatttctacatattttttctttttttatttaaaattttaaaataattaagatgggggggaggatttcaggtgaagaccggaaatcccatacagtgctgaaaaggtggaaatcaccaagaaaacaggccacatgtaatgccaccacccattgctatgtcgataaacatgcttccaaggaagttcatctcagtattatttgtaattagcaacaacaacaaaagaaaagaagctaaatattgacattgcagggttggctattaataagatgagacatggatgctgttgtgtaactgggctttccataatgggtttccattgtgattggatattcccctttaagaccaaatctgaattgttctgttctgctctagccccacactttgagggaggtgttgacgtcttgtctgtatccaaagcagagcagctgggaggacaagaggctggaaatcatttcctaggagggaagctcagagaaactgaggtgactggagccagataggacctgaggggtgggcagcaagatcaagtgtctgaaaggccacaggctgcagatcagatcaggccagtatagtcccatgacaggtagaaccagaactgatgcggaggctgtgggaaatgaatttcagctcagtgaggaaacattgtccgtgctgcaagaatggagcagacctgcccagggccaccagaagaatcttccttgacctgatcacatcctttatctggaacggttcctgctgcttatttgtccattaaagaaaactcaagcgcttagcctggcattctaagccctccctgacatgtgcggtctcattccaggaatattttccactccccccagactccacctggtgcatgtgggccatgcccccttccccaggcaggctctgcattctgctgctctgagcctcagcaaattcccttctctctgctgtgtaccactctctgcttctcatctcccctcattcagtcctcccctgccgcaagtcccagctcctccatgatgcccgcaatcagaaggccttgccccctcttctaattcctagactgccttatgggtacctctcccaggacatggccacttccttcccagctgtagccccaggtgtatatgtgacccttccctataatggtcagtgcttgtgagaatatgggacacccctttttcatcctctcatccagcatgcggctcagtgccaggattctaatggataaatgtttccagagacttctaagggggaagctaaatgtcttgttctttcctagtagctctccttcttgcatttatttttagctggatgtttttatgcctccaattctagtttgcccattaaaccagctcaatgggttagggaggacattgatcgtcatccctattgtacatcaagagaaactgaggcctagagggtttaggtgacttatttaaggtcactcacttagaaagcggcaaactccacctggaatctgggtccagccttttgcctctgatgcatcctgatttattctccatgtccagcagggcatccgtcctcgccacctgtggtggacaccgtgcatggcaaagtgctggggaagttcgtcagcttagaaggatttgcacagcctgtggccattttcctgggaatcccttttgccaagccgcctcttggacccctgaggtttactccaccgcagcctgcagaaccatggagctttgtgaagaatgccacctcgtaccctcctatgtaagctgcggcatgtgtccttggggatgtttacctcaaagtgatgcaggaaggagtcaaggcagtcccctgatgggctgatcctttgctctggactccttaagatctttgtagatccttaagaacattccagaactctcacagcattctggagtccattatttaacacatgtttattgagcacctgctgtgtgccaggcgtgttctgggttcttgggatccattagtgaaaaagcaaagatccctaggttcctggagcgtgcattctagcagggggagacagataagaacaatatgcaaattactccgtggattatgtgatatgatagaacaggaaaagtgccttggaaacaaagaagcagttaagcaggtgagggagatcagaaggaaggatcccatgaattctgctgtcttgaattttgacatagtcggggagaggagtgggccaggaggtgtgtgggtggcaagtgtactgaggtgtccactcggcaagtgctcaactctcaggtgtgcctattcctgaggagaacaatggaaggtgtgtccattcaccctggccaagctgggaagaaaagcccaaaggttctaattggcctcaccatcactccccagggtaggaaggaccctacagacatcagagcagaccctgctcatctcagcagccaggtgttcggaggctttccagcagcccaccagtggccgccttcttttcttcccttttttaaaaaataatgtttactgtttttattttctgagtgcaaatgtaatatatacttattacagaaaatttaggaaatacagagaaaggaaagtaagaaaattaagatcacatacaatattccaccacccagagagaacaactatcagtattttagatggtaaagtattatgatgtaatatattgtttgtattccagtttttttgcctgttgatatactttctttctttaaaagggaatgctatattgagataatttggatgcctgctttgctcatttataagtatatcatgagcataattccatgccattaaatagcacagcctggtattaaaagctgcattacattgggtattgaaccataattaatttccccaagcttttatttggggtcattaagttatatatgatgtttttaataatataacatcatgaatgtgaaacatttatgcacattcatgaatagtttaggataacttctcagaaatgacatttctgagtcaaagagtatatatttcttgtttgatttccctcccaaaatattccaactacactcctcccattgacgtatgaacatctccatttggcagactcttggcaatgcttgggattattaaaaatatttttggctattgtataggcaaaataataataacaataccagcaatattattttcctctttaatttgcatttatctgaagaccagtgagttcttttttcctttcttcacattggaatgtattattattattattattattattattattgttattcctcttgcacttggtgatcttaggaaaatttcccaggatggaatcaagaagtcatctttgctggctctgattctgtcacttgagagctgtgtcatcccagagtctgagtgcctcagtttcctcatgtacctagtgggggcggtgatgactgccttttggtacattgaggtctgggactgttgcttataatatacttgtgtataagcaccacatggcatggatctacctggctatgtaaccttggtcaggttctctaacctgcctaggcttcagttttctcatctgtaaaatggagataatagaacctacctaatgggactagtctgaggcttaaatgagttaatatacaaaaggtacttaaaacagtgcctggaacactgggagaactaaataactattaccagtgttcatctatgttgcatccttgagatctttcatcagggcaacacaacagcaactgcctgggagaggcaggtggtagaatcttggcatgtcagggctgcaagactcatgtagaaatcactccgctcacttagacctggcggattgggacgcagagtgtaaaaggaattcacccaaggtcaccccataaatgcatgggagagacaaaactagagcacaattctcctgatgttgtcccacaaagatatcacccctgagctattgtgagaaatgtcacctcccagggaggatcagtgtattagtggtctcccctccttgatgctgggagttccaaaggctctggaaagggaagaggtgtgaagcccttctcactctgcttggtcttaggagaccttagtgagtcccagcgcccccacctgaagcccctgatagcctcctacccactacaatgtcgtgagtctgtaactatcaagtccatttccaggcttaaacctcccagtccaaggcgctgccgtagaaaacacctcgcttagatctggctgaacttcaggggttcttctttccccctccccaggtgcacccaagatcccaaggcggggcagttactctcagagctatttacaaaccgaaaggagaacattcctctcaagctttctgaagactgtctttacctcaatatttacactcctgctgacttgaccaagaaaaacaggctgccggtaagttgtgggaccccctggtcaaggcgtgtcagtgccagtaccccgcatcttctggggcagaatggaagggagtgaaggcagcttggggagggagctgcacaggccaggattgtttcagagaccccagggcctccacaggaaacactggttttccacacatcagtttaccctcgtgacataggcactctgggggagtttgttttgcatctttaatgaacatcttaattgttccaagttccctcatgctaatagaaggatgagaatgattacttttagttactagattaaaaatctgataccaagattgggtaagacatgattttcaatgttagtggtggaaagaacttagagaaaagccaacaccccaactcttccttttcaaatagagaaactgaggcccagacagggaaggggaggtcacccagtagggtagttttggctcaagtcttctgatcttcacatcagtgcttttgacatggagtagggcattgagcttgtatgtggcctttctccagcacagcaacattcatctggggtaactttcactgtccaggtacggatgttgggggcggagcctggacaatcccagaagctcttccttttgcctttggccaaatagctgggatttctttttgtacctaatttccctccctaattgtcttggcaaaggctgatgacccagtgagtcagaatttcccaaataaatgaagggcaggacatggggcagcccgccagctgcaggttggagggtggggagggtctgctggcaaaggaggcacccagggtgaaaaaacaaggggtgttgtgtctagccagtctcccaggggcaccgtggcatggtgcctaggagtgggggagcaggtctgcgaacgtggaactagaagttggtcctacatgtgctttgtgagacacaggaagtgagagaaaggtttactcattcattcattcattcattcattcattcattcttctattgtgtactcagtgccccatatacacagttagacctggggcttcagggaaaggaggaacatgaatccactccacacagcccctgccttcaaggatcttgccttctgggctgtagagacatatcctcccttcagcactttattgcaggccagccctcaggctcagtcttcacacaaagatgaatgcacactcaatccctccccaggaggcaggcagggattagagttgtgggagaaacaagggctctgggaacctggagtcaggagaacctccaggaatggccacaggctctagaactacacggtctgggtttaaatcctggttctgcttttgactagctgagagacttgggagtttcttaacttctctaagcctctgtttcctcaaaatgggaatcatcacatatgatcagtcaagggtgaccactaagcgtgatctcagtccctggctaaaaaggcagaggcaggctcatacccctgccctaacagtggactcactgcactgtggccggcgcctgcagtgtgctgggcagctcaacatgtggactctggaggcagactgcctgggttaaagtcctgctgctaccacttttgagttgtaggaccttgggcaggttaagtgcctccattttcccacctggataatgggacattatcagtacctacccaatgtatagtttattttactgtgcttagaacagcacttgggacatagtttatgttacgagttcttttatatttcatgaataatattattacaatatcattattttagccactctcacttcttccccactatgtggacttttaaaatgcaagaatcatgcatgaggaattgtatccactctcatcctccagtattagcagagtccctggcacatgctaagttctcgacagtgttagtccattgactgaaggcatcctgaagtgggtgggttttaggctgggttctgcagggtggataggatttggggatgcagaggcataaggaggcatttgaggaagagacaccatcacaggcacgtgtgcttctcgagacaatcattcatgcacagagaagtttacttcaccaagagatagttaactgagatgaagctgagatacaagggctgccccatgggtacgctgagtgcatgaatagtctaggcttgagggtgatgggagtgtcctcccgaagaggacatccttagcctttgctatgcccatgatgatgttctcagcatcaagagcctttcggggaggaggcactggaggctgggtagaaaggaagggacagccagggtatgtgcagtagggtggtggcttgggtcccagttgggagaattatgcaggagagagattgccttttgcaaagttgctgggagctggtgaactttggatagcatcctccttcagttgtaaccaacaggtaggtcaccaaacaagacatcttctgagtgctccctggctctgtgtcctgtaactggtatgttgcttcctctctgcccaggtgatggtgtggatccacggaggggggctgatggtgggtgcggcatcaacctatgatgggctggcccttgctgcccatgaaaacgtggtggtggtgaccattcaatatcgcctgggcatctggggattcttcaggtaagaaatcggactctcctcactgcactttggcccccagaacgaggatgctaggacccagctctggtcatgccagccctcaggggagcttagctaggttccacagtaaggcatccaagccccttcgtaattggacactacctaccctctcactacccagccactcatccacttgcctctgagcttttgcatgtgctgttccctctgcctggaatgcttattctatccgaagaacacctcttcatcctgcaagacccagcccccagttacctccactgaaagactcatctcctcttcacctatgtttctccagcacattgcttttctctgtcatgacacttagcagtcagcccagcaggtgaaggttgacacatctgacttctcatggaaaggagggaaggggcagagtcacagagggagctcagggcatgttgtgggtgaaggaatgggggctgcagatggtggggtgtgggagcatctgaccccttcctgtgggagtgttgggacccactcagctcattttctgcttgaaatttggcaagtgtgggaagcaaggatggagtcttcccatcaggtctccatggggacaggagtcagatctccttcaaggcgcaactgctatgaaccttttctaaggttctcctccatccatgccagtcaggtttcttcctgaacacgccactcagttggtctcattgcggtcatcaatgacctccccctgctctccacccaacctctcaggacctcccagcagcagcagatggccactcagcctctgtgacatgcttccttcccagcccctttcctggtcctccctcctccctgtccacaggcctctcttgtctccctctccctccttactaattcccagtgatcccagggagcagccagagcccctgtttccagcccacctcacctttgaagcccacaggcatcactcactccctcctccccacccctggctgtctaaagggtgccccactcttcccagggacaagttgaatccttgactcccactgcccctccccaacagagacccccttctcctcccaccacgcctccactcagcttctgctgccacatcctcccttgggctcaggagaaggccttgtggacagcccggactgtctcttttctcacaaccctcttggctgcgccttcagaatccactcagagtcccctactttgcacctcttccctggctcctagcccagccaaagctctctgtctgtcaccttgattatgatctttaacactttcagatccctgccacttttatataaagctgagttgcacagtagccagagtgatcacttaacacatgcattgaatcatgcgatgcctctgcacaaaaccctgcaggggcttcccatgctccttaccatgacagccgaagttcttgccaggccccagggccctctctagctgcacctcctgactcacactttctttcactgggccccaggccctttgcacttgctttttcttctgcctggaattcctttctttggatatttacaaggttcatgtgctcattgtgcttgttttatgaaggtctttgcctccaaaacctcaaatgtcactgcctccaaaaagccttccctgattgggatctgaaatggcaccccctgtcccactcctgtttttttccttactccattgtatttacctcagtgcactttgactacctgacatcattatattctctctatgtatctgtttatctgcttgtgatctgtagtgggaattcagtgtcatcacaggttttgcctgttttgctctcagatgtgctctagctcataagacagcgtcaggcatacagtagatgctcagtaaatagttgccagttgtgtgaatgtagaaccatacatcaccacaatgctgtactaatgaaagcagggtctcagagatgctgaagcccagccttattcttaagggttcactgagaacccctagccccatctgtggtcctgaaggtcctgcatgacatctctgctccccaccctcaacctgttctcttcctcacagcacaggggatgaacacagccgggggaactggggtcacctggaccaggtggctgccctgcgctgggtccaggacaacattgccagctttggagggaacccaggctctgtgaccatctttggagagtcagcgggaggagaaagtgtctctgttcttgtgagttttcctgtcaccaggcccaaccccacgcttgatgtgatgctgatgagacctcttggacacctgtcaatccagctatggatacctctgattacaatacctgaattcaggactggccctactcacagcccagcatcagggggtagaaagctaaagaccagctctccttggcccccaggaagctcagagctcagcttagtgtcctggggccatatgaatctccagttgcagcctgcaatggtggcattctcctcttccagcttggttgagctctctctctctctctctctttctctctctccttcacactcattttaacataaggactcacatcccttctcttgggaagtaggaataggcataaattatgagtaagggcaggcagagagaagaggtggacagaggtcattgtttggctaaaccagacctacatgtggaggggacatggacactgagcaggctgggaattcctctggggtggtctgatggcttgtccatgcccaagaaggaggcgacagtctttgtgactgtggggtccagtgggctaggggaggcaggcagaggaaggagggatggagccgcggatagggagggatggggcaggggttgtgggtcatagacacaaccttgggtgtgaggggtcctgctgggattggggattgggttcaggagacactgggggatctgggatgaaaacccagatgagaggtgctgggagctgtaggaagacttccacctccttgaggtgggcagagggtcagcccactactggattcctcagtcccgtgttggttttatagtggagtagatctagcctggaatagcgagtgagtcactgaccccactcctgagcatgaactctcctcccctccactctgctgtcaggttttgtctccattggccaagaacctcttccaccgggccatttctgagagtggcgtggccctcacttctgttctggtgaagaaaggtgatgtcaagcccttggctgaggtaggtctccggctggtacgtctccggctggacacccccacctccttggctctatgctcctgaatcctcagggatctctcttgtggtcggttgtagctaatgttctcctagaatcactgaggcaccaatggctgagcaggaagggcgaggagacaccttgatcagcgtcccagtttcacagccaggcaaaccgacacagggcttggaagggatttgccaagggcagcaggtgatcagggcagagctgggactccagctcatggccctagcagccagtacagtgccctatctgtgaccacactcctcctatgtgccagggcctggtgccatgttgggcagtgatggtgtcttgtgtctctctgggtctgcctaatggctggtaagtggaacaaccaggatttgaaagcagacaaggaaattcaggattccatgctctctatcccagctccacctcacatttcttgacacattcaccttgagatgattatgtccatttcaatggagataaggtagcagagatgagagattacataattgacccatgttacaattgagattagtaacagaggcaacgctccgtgggacctggaacccacacccacgggtctacagtatcttctgctgctccctgccactagtacaagttgggcttgggatagaatgccactttcctctttgatggagggaagggatgtcgctcttgaactctgttgttgcctgtgatctttgcagcaaattgctatcactgctgggtgcaaaaccaccacctctgctgtcatggttcactgcctgcgacagaagacggaagaggagctcttggagacgacattgaaaatggtaggttgcctgttcccgtagcccaaaccctgtaaacttggtcccagacttcttcatttcagctgtcctcttgcccagggacagtttcctgggacaatttctcaactctcagtatctgaatggggaatctgatttgtccttttttattgtaaaatgccacaaatgtaaaaaaagaaagtcaaaaaataacatgataaaaaattgtgtaacaatccccagtcctgaccaaaatttaatattttgccattcttgttttcagatgtatttttaagaaattaaatgttacatattcccaggggacaccatcatcctgaatttgggggatggagatattaagctcaaagattttcagaaagatgtcacaatttatcttggttgacttagaaactgtctgtattagacctagtggtggtccagttttctagattttctgagactgactcagttgtcatcctaggatagtcatccatccacccattcgttaatccgtctatctatgattgtcttatccatctatctgttcactaattcatccaattcactcatatctttttatcaatctaatgtcaacctattcataactttgtatttatctattttcaatccattcaccattcatggatcatccagccaccttatatctcaactccatcacccattcctccaaaatcaacaatccaattatcgcctgtctgctagttttcacccatctattcatgtatccattcaattcaactgtactccatgtattgaccaactccatccatccctccattgatccatccatccatccatgctaaatatgtaggggtgggtgttagaggtagcaaaacagacatgaagtggacatagtccctgctctcaaggaactatccaaagagaaatacattcatatacttcgcaggttgagtatggtggcagcacagaggggaagcattcattgtagtaatcagggatgcttcacagagaaggcggaggagccagatttgagaccagacagtggaattcaggagttcatgcccttaatcctgactccaccttatctttcctgagaaattcagctttcagatcattgtgcccattttaacagaggtaacaaagacagataaattttgtaattgccccatgtcacagttcaaattagtcacagaggcaacactgtaggacccagaaccgacacctgcgggtctagagtatcttctgccttccctgctccctgccatcctcacatattaggacttggggacctttaggattgttggatggccatggcagtctctcatcacagggaagaccaggaggacaaacacccagatgacacagcacccagggccattgggaactattccctttgagggggaagagtgtgaattccaagctcaggagtagcctggactctctccttggacctgaggctactcctggatcccagggctggcctctaccactggactctgcttgtgtttccatgagttgagtccaatgtggtattggtgcttaggtcttggctcaggctctaagtgaccaaagtgttcaaggaatgaaaacacaactgttttctaacgcccggaaggaggcaaatattccagcaattctgcatgtggcatcagaggacccagcttaaaagggaagagttgagtctttgggaaaacccatccctaagatcctagaacattcttttgagtttttctgagatcttgtggaagcacctccatgattacgcgctgcctccagtacacacacatgcagacacacagacaaacacacacacacacagacacacacacagagaaacacatacagacacacacacacagagacacacacagacacccacacacaaagagccacacacagacacactcatacacacacacacacacactcctggctagtgggacttcaatccttaatggagggacctgcactggttacatcctgagcacaggtcagatctgggaactacagtgcaacactacagtcactgcaatcttggtgaacacacaccaaagagaagcatggggtaggatgagactcagtgagcttgcgtgagtcagggctttctaatgggagatgaggaaactctcccaagtaccttgaacagaaaaagaaatgtgttgcaatagcatcgtcgtgtaaactgaaaatctcaggagttgatggcttcaggcatggctgggtccagatgatcataggatattattgagaatctaccattctctgtccttcagctttgcacatctctgtgttggcttcactcttaggcagatatatctcctgggaaggttaaagacaacagcactctagacttatgtcctatctgcttaaaaaccctctttctaaacaattcctgcaaaagttttggggtaaactctattggattgacatgagtgtatgcccatccctgaactaatattcatgttcagggagatggagcaccgcaggtcacatatagatatacgaattcacggagtgatgcgggaagaacctgacacctctgttgccccactcacccagctcagtgttctcctggtaagcctctcacccacatcctctgcctttgtcttcacagaaattcttatctctggacttacagggagaccccagagaggtaaggaccttttgtttctggattacgggttttgagtcttagcacctttaagctccaattaactgtgagtgaaagaatcatctcttgggtaattatagtaactcctgcgtgtttgtttctgaggcccagagaggggtagtgactcacctgggtaacacagccaggaagactagtggctggcctggaacccattttcctgactcccagtccagtgctcccaggcatcacctctgtgtgccctgggctctgcccactccccttcttttttacattttctgctccccaaaatggcactgtaggaggaggctgaaacagaaccttccacaatcaggaggcagagataacagcgatgattagccaaggagaggggaagcctaaatctcagtccaaggacactcgtctcctcccagcacacaggaaactccaacagtattctcccaatctcctcatccccactcccacccccacctcccagtgggttgacagtttctggtgacatcacctctgacgaatcttacaatcctgtcctctctgctgcctccctggagatcacagcactctcctaaatggtcatgggcggagtacatgagattcattcagcaaagacttaataaacacttcctatgtgccaggacttgtttaggagctggggatataacagtgaaaaaagaaagactccctaccctcttgggaccttccattacagtgccaggacagtgcaataaaaaagcaaatcaagtatgtattccgtttgatggtgtgcagcattaggggatagagtttgtgggtggcttcaatttaagtagcaggatcagggaatggttcaatgagaaggtggcatttgagccaagctctggaggaggcaggaagccagctgtcaggaaacctggagaagcctattccaggcagagggaacagtcactgcaaagaccctgacagaaggggccagtctggctggagaggaccgagtgtggggacagactggcttgaggctaaagaggtaatgggcagtgggagagagatcaattaaagtacttggatttggattcagagcaagatgggaagcctttggaggttttgaacagaggagtcacatgatcttagatctcacaaaggtctctgtctctggtgttcaaatagactgtaggaaaggggcaaagtggatgcagttgaccagctgggcagccactgcattgccataatccaggcaagggctcctggctgcttagacagggctgtggcagtggggatgggaggagtagttggagtctggatatctttgaaggaatagctgacaggatttgctgatggacaggatgcaaggggtaagaaacggggaggagtggaggacgccctgagttttctgacacaacaaggttgtgcccacacagggcagccacttgcaaactctaagtggagaacaatcacttgtcattgttgctgatggcagtcttccagcatggctgtcacccaactgaggtgctcccgtctcttgcctcaacttttagcctcttctacttcaccttctctccccatttccatgtctcatggtgcagccagactggccttccttaacagaattagagcacgtcacccccacttcttaaaagcctgaccttccttatgagacaaatccccttccctccatggagttagtccttcaaaacacacctgcgtgcacactgtgtgccagactgtgcagaaggtaggctctgcacctgtccctccctctagaccctgccccacagcatttctccaagccaagaactcttggttgtttctggaatttaccctgcacagtgacacctatgtccttttgctcatgcggttcccactgtttagaaattctcactcctttcctcacctggcaataccattcactgcccagagcccaccgcaggcaggcggcgtctcttccacgtagtctgccctgcatcttccttccagaggggctgctcctcttgctggcctcccacagcagccctgcctgaactgcacagcctcttaaggaggctggacttgcaccccaatcctgttcttcattgtcagccatattggattaggatctgatcttgtccagctaggtctggtccacttttcagaacatctcccccaggcagtccgaactagctcgtcattcattggcttttgtagagcagaccaaaggtcccagaagccatagggtctcaaaggctaggaattgtccagttccatctgatatccagaagggaaatacagcccatggggcgtggaactgggaagattccacagaggactggtgtctatgagagggacccacagcaaccttgctaacaagtaatacctaacagttattcagcctttccagacaccaggtgctgtgctaagtattttacatgtgttcagccatttaatcctcacaataactcacctcttagatgagaaaactgaggcccaaacaggtgaaactcccagccagtaagtagcagagccaggcttcagatgcagttaatctggtttcaaagtccatgctctatcttagcaacatccacgctctatcgtagcaactacaccattgtgactaaatatgaaatataactgtacccagaaccagctgccctgatggcaacacatgagttgggctctctctaatctgtgaccatataaaaattattcatcaaaggtgaaacctaaaattaagacatggatcaatatactgtgagttaatcactgattcttttactcatgattttctctctagtagggaatcatgtcctagtctaggctccttgagtgaccagggttcggtacctcctcaaagccacccgtggatcaataacagctcttgtttaaatacagatagatagcataatctcttcctcattaatcatggattctgtgtttataaattcacctgcttgctaaaatgtccttataacccccaaatcaatactggtggcactttcatggtcacacacaggcaggtacagagcaggggaagctggatttgcatgatgggcatgttccatctgagatccggcgaggcaacctctctcttcttgtttcatctctgatgtttacaagtgtcctcttcgcggtctatttagtgccgtgtgttctgcatttttgtgctttttgtagtgatgtcactgtttagagcgaacccaagagtagcgctgctgtctggcgttcctgagctaaacaggctgtgctgtgccttacggggaaggtgcctgtgtgagacaagcttggatgaggcaggagctgcagtgctgctggccgtgcatttggtgttgaaaaatccacataatagcacattcagaaaaaggagaaggaaattggccgattcatacatgaggctgtgatagaaagggctaaagtaacacctattgtgtgggatggagccgtggaagtctctttcaacagaaacccacacataaaagaaggttgatgaaaatgttgtggccagaggtttgcaaaaacctaacccagtatttcccccttgagtgatgaccctgtattctctaattcagtactcacgggggctttatcagaagcaactgccaggactaccgaaaatcaattgtggacacacagaggaaaatgcatgcacttcaaattgaaatatatagacatgtaattttacatagataatacatgtatttcaatatgagtgaagatttcttcctctcctcatcacctgagcagtgtcagcctctgcctgaagttctgcccctgagtcatggagacctacccccctaagttaggatcctgagcatgtgcagccatggcgcatggccatgccggtctatggtactggtctcaccctcagacaggcagagttggggacatgggtgtgactagggagggaatcacaggtgctgcactgctctctccccagagtcaaccccttctgggcactgtgattgatgggatgctgctgctgaaaacacctgaagagcttcaagctgaaaggaatttccacactgtcccctacatggtcggaattaacaagcaggagtttggctggttgattccaatggtgagaaggcacatgtctgttggagggactcaaccacccccatcagccctcctgtctctggtcccaggaatgcttccctctccgagctgcactctgagtcctgggcagctgttcccttcagcaggagttaacatttccacggaaatcttctccaggagggcacagttttcaccggggtatcagggccctgggattccctctccccagactctcttattgtcttcctattgaagaatcctgaagtgtctgtgctgggagggccagtagagaaaataactctgcagatgggaaaacggagggggcctagaagggggaaggagtggaggagtccggagaagcagtcgggggcttcctggcttcccacctcgggctgggtgtgcgtaattcccctgaaaatcactcatgctcttcatcacctcattgaaaatatccaaagaaagtggcttcttcttgagttaccattgacttctagggaacagcagacatcagcgtctactaagagagtggatggtcagaggaagatgaggatggaaaagctacctaatcgggtgctatgctcaatacctggtaacaaaataatctatacagcaaactccagagacccaaattacccatgatcaaacctgcacagttacccccaaacctaaaataaaagttggaaacaaataaatggaaaaagtaattgagttctaaaagtaaagaagtgttctccttccagttgaagataaacaaaactgaccctggttaaagcagtaggacagatttattcagtaatatgattgcaataggggaaatagtccagcgtggagtggactcacatccctgaaacaaaagcctgatgacgtttcataggccaggtgtgccaagggaaacactgtggtctatggggagaggcttggttcatgtgactagaccacctggatgtcttcatcctggctcacctggggcagaaacaaactcctcttatctttaggacaggaagccatgcattagaccgcaggaagaacccactgaagtcaggctctactctttcgacagggactgggaagatcaggagtgagctccctgaatgtttgcattgcaaagagaaggctctcaggtcctcaaggaaatggggttgggtggtagattcacatcccaaagggcagagaaagtgtttatcattgcaggcttctaaagtaactgctctagttgggttcggggacctggctgcttgtctccaggttttggctggagcagagtaaattgtcctggcagggttgagccttcccaggcagttatgttaaggatactggggtcattctagggacacagccttaagctgctagaagcaatgctagagattggtcgagtccctgagtgcagaggtttgggaagagttgttatgtgcgtagagatgtgcagttcgcccctcagaaggatttcagaggttataccaaagaccaaagccacgaagacaggatttaaggatttcagtgtcatctgtgacaggtgggggtactagggaggtctacatcctcttccccagagccctctattccacactggcctggcagacaccttgcatctccatgtcactgaccactgtagggatcccagccacagacacacacacacacacgcgcgcacacacacacactgcaaaacactgccacagcttctctgaaattttgttttttgttttttttttttgtaaactttgcaatgcttgagtttctggatataagcccatttgattcattgatttcacccatttcagtaaagactcatgcccttaaaagcccccataattagaggactttcagactgcattggtttggttggttggtcagtttgtttcttatcattaattcccaatgattcataaatgctgaactttttttttttttttagcagttgatgagctatccactctccgaagggcaactggaccagaagacagccatgtcactcctgtggaagtcctatccccttgttgtaagagtctaggaatcatgggaattggctgagacccagagagggacaaggacttgcccaaatcatagagcaattaaatggcagaatgaagactggggcctcagggtttcccccatctttccacctttcccacttcattttccaccctagcggggagttgcacagggcttatggggttcaccattgaggcaggactttctggtgggctggagaagctgcatcgctcacccggggctggtggtcactttttgatctatttcagtgcattgctaaggaactgattccagaagccactgagaaatacttaggaggaacagacgacactgtcaaaaagaaagacctgttcctggacttgatagcagatgtgatgtttggtgtcccatctgtgattgtggcccggaaccacagaggtgagtcccagaggtcgaacgggagggacacaaaccccacgagcttctgtatctgacccacctacctcccagcatagacagacatggaaacagctgagggtcaggcctcaaaggctgattccatatggcatgggatgaggtgctgtcttattttggtttatgccagcagaagactgtgagaaaaaaaaatccagaggcaagtggtttttttaaggtgatgatcccagataacagcagtagggaggtggggaagtgagagaggaaagagatggaatccaatccaagatgcgttgtcaagggagtatccactgtggacaactggagcggggaaactcaaggaaacaccaagaatacagggctcaggggcatcccatctgagtggcaagggagccgggtatttattcaccagcttctacttgtcattggttgaaggctcttgcaaggaattgtttattccctgtgacttcgacctgctgcacacaagggcagagtagtctcttgtgaccagacaaaggcctcaggcctggagctgcagatgctggaggtggaagtcaggctggcatgcccaggaagaggggatataggtgagaccctggcagcatctgctgcaagggctccatgcctggagttattgtgggacatagggctgctgtaggacaaatattgaaagatgagtcgtggaagggttttaaggtctgagccagctgcaaacaagtttgttaagtattggagctggataaaaagtgggagccctgcatgggatccagaggattgtccaggaccaagaaggccagagagaggagccagggcgggtgtgggagaggtggtctgtagaatggcagtgtctgagggtggtttttatgcatctcagtgcgtgcagtagcatgcaagcacagggcaagggcagggtctatgcaggacctgctaccactgacccaaggctgtgtgggtggggcatgacgttagggatgtgtgtgctcacctagagaggagtgtctgttcctgcgtgccagctagaggagattcacaggattccttttcttccagctctcattcgcctagtctgcaagctaggaaatgtcctctctctaaccatgctggaagggtttttaaatttagaacttcataagcatgataaaaactagctaacgcttaactagcaggtgccctgacacacctttgcacaggaaggggcaggtgctcataaccctcatttcttcacagggtcctgataaaaacctttaaccaagaccactggagttgaatctgttctttcttaaacttgcccttgccctatgctctgcgtctgaactatatatagagttcccatctccattttctgatggtttgagcaatactgaacttatgtttttcatctggtttcccatgactttaagttcagggcttttttttttttttttttttttgccaggactacacagattttatctactttcacttattgttgtctcactctttttgataaaataacatttctgaatataaaattaatatgttaaattatagaaactttcaaaaatatggaaacacaaaaagaagaaactatgtatcactcatttccactacctggaggcaagtgctataaattttttgtgttttcgtctaaagattttccatgtcctatatactttaaaaatcaattttatattttacttttgttcatagaacactatgttgtgaggattttctcaatgttgatacaaactcatcaaagcattccttattccatgaaatggctgtgttaaatagactttaatattcatatcctattagatgtttagcctggctactcacattctgatgctacaataacattgagctcagcctaatgttcataagtctctaaacacatgtctaataattttcttaggattatttttagaagtaaataactaggttggacaatgcaaatgttttagaactttgggcaaatattgtaaaattgcattccactgcataagaatgtggatttcttaattctctttctcatttgggatgctaagattaaaaattagcataacacttccagtttcataagccttccccaccaacaagaagcacatcagcctgttttaatctgcatgtcctccatgactagccctctcattgtctctgcttctcttattatgtctaattgtcattacccaaatctgtgcgctactttcctctgaagacttgtgttgtgattgtaagaattgtataaatatcaatggctttagccaaagagttttacaaagtacgttttgggcagcatgctgcctataataagttctatcattcacaaaagctctttcttctatggttggctctgagtttctttggaaaatgttcgaggggcaaaggcaaggtcaaaccctccttttagcaagttttgtttgaccttgaagcagccatttaacaggtggattacagagccacagaaggatggcatcttcccaggtgcgcttcctgctgagccgagggtgcagtgcaggaggctgacaaagatgagtgaatagattattcttcattctcacattgaaatgacatgtgcgggtggggcatgaggttagggatgcgtgagctcccattgttaaatccttttgtgaacgaatttttaatgatgttaagaaatgatggctggaggcggtggctcttgccaacactctcagcattttgggaggctgaggtgggaggattatgagagcctaggatttcaagatcagcctgggcagcatagtgagaccctgtctctacaaaacaatttaaaagggaagtgattacagtgtactacaaaataccatatataatatcatccccagttaaatacataaaagagagatatgctaaaataaaaaagaagcattctctgagtggtagagttatgagtgatactttaaatttgattgttaaaattgatttttaaattatataggatatggaaaaattttagaaggaaatacaaaaaatgcatggcccttgcctcaggtagtggaattatgagtgatatctagttttgctttatgcgtttccatatttttgtttcttctaattattcctaattgtttttattttccaaatttcttactgtacccaaaggggaggacattgtaaattttctccaccagcctggcattgccctgactctttccgtcttagtgtcacttctcagtgggtatcatgaccagtgcaaagtgctgaggcacagggccaggcggcccacgaggtagccctgaaggggcagcgatctgtggctccaatgagccaatcattgggaaactggcatcttcctggggctgggacaggagagctttgttggtgggagaggagggcagagccgaaggagagagggcaggggagaggcaaggtcacccttgctcagcgccttcttttctctgccagggctttactgccttaacaccctaacactcccaggccaggcaggcagcaagacacatggggacagtgagctataaagcagtgagtgccgcaggttcctgagacacaagaggcagattgttccctctctgcatatgtttgtatttaaatagcctcatgttcagacaagcaacttggatttgcttcagtcttctcaggatgggtgagaaaaacactggaaatgctcaaaacgaggcagaacagaagacacctgtcatggtggagatggcatccaagtgaatgaacgcgagtctgttatcaatgaattatatccggtgtaaccagatgtagagagattgtcttgtgtgagaatgcaccagcaggcaaaggggtttacgaggcagagtcagggtccctgcaccggcagctgttttaaatgaattgacccagggtttccacattcattcattaaacatttgttcattgaacaaatatttggctctaattccgcttccacctcagacagctatgcagcccggagcacatcactgagctttcctgagactctgtttctctactgtgctgacagccagaggtttgctgcagagctggagaccttggtagcctccaggtgtgtgtctggcaacatgaactgtgcatgaatccttgtcttctcagatgctggagcacccacctacatgtatgagtttcagtaccgtccaagcttctcatcagacatgaaacccaagacggtgataggagaccacggggatgagctcttctccgtctttggggccccatttttaaaaggtaatgctccttcctgtctgtgagctaggagttagagacttgggttccagtcttggtgtggtggccttcagcaattttgtcctctcctggtcttagtttcctgattctgtataacagggttgaatgtcactgggttgtccgccggcttcctcccactctaacacatgtgaggagggatgtgtattcatgtgccctctgcacagacgctcagaatcctgctgtgtgacatcaagacaggtggtggtgggggtctcctgcaggattcactatgacagggttgaatgtcactgggttgttccccggcttcctcccgctctaacacacctgagggaggatgtgtatcatgtaccctctgccacagacactcagaatcctgggctgtgtcatcaacaagataggcggtgggacacatgcggggccctgctctaggttgagaagccaccaatcaatttccctctcccaggagcgctgagacagtgagtgagggtgagattagaattggtgttaaccctaagtcaggtctaatgtgtgcctgtttgtctttctgtctgtctctaacaaaaactcataaacctttacatttagtacatttaataattgttgatttagaattcacaggatgtatgaatttggaacaggagaagagaatattttttatatggtttatagtcagcaaggtggccatcccacaggctgggaaggtgcctctggccaagcccagaaacgggctcattggaggaggggttgggtaggagctttatgtgaacggattggctaaacatacatgttcaacaggttacagggggagcaatggatattcatgaagacagtcctgacacatgtgtattaaacaaacatgtatgtaacatggcccatgttcacctggtggtggagacctaatatttaaatgtattacaattagggcctgtaagtccaaaggtcttctcaggacacgaagctcagcaagtgaggagcttctgtacaccggccaggtccagtccatggctggtgatgttcttatctggagaaagttactgaaatcagtctcttatgcaatcaaagcagtagttaagtctggcaagtcagggttctgtttgtctgcgtatcccagctgcagccgttgttattgttttccttttgcttagctccaggccagtgctggtttagctgctagagaaagagaagcacctcgtggcagtgagaacaaagtggattcttttttttttttattattatactttaagttttagggtacatgtgcacattgtgcaggttagttacatatgtatacatgtgccgtgctggtgcgctgcacccactaactcgtcatctagcattaggtatatctcccagtgctatccctcccccctccccccaccccaccacagtccccagagtgtgatattccccttcctgtgtccatgtgatctcattgttcaattcccacctatgagtgagaatatgcggtgtttggttttttgttcttgcgatagtttactgagaatgatggtttccaatttcatccatgtccctacaaaggacatgaactcatcattttttatggctgcatagtattccacggtgtatatgtgccacattttcttaatccagtctatcattgttggacatttgggttggttccaagtctttgctattgtgaataatgccgcaataaacatacgtgtgcatgtgtctttatagcagcatgatttatagtcatttgggtatatacccagtaatgggatggctgggtcaaatggtatttctagttctagatccctgaggaatcgccacactgacttccacaatggttgaactagtttacagtcccaccaacagtgtaaaagtgttcctatttctccacatcctctccagcacctgttgtttcctgactttttagagatgcaggcctgaacccttgcccgacgtggccttaggtctcgtttttaatttggtgtcttattgccacaaagagtgtgttctgtcagaatgatgaccttcattttattgctgatgctggtccggtggtgtctaaatcacaaaagggagggagtattatgaggcgtgtctgacctcctgtccgttcccaggcaggaacagagttgaaggtttttcgaggctccccttagcccagagagggtctgttcagtcagttgggggtgtcaggattttatttttagtttacattatacaatagtttaaaaaatcctttcacggtgtcttctaagtcttaactctcagcagttacagatcagatgggactcaccccatggctgctcagaacgcaccagcgcctctgggcatctcactgtgcatgcttaggcgccttgcggctctgttgtttttcagaatgtgaattgtgggtgtgtgctggggagggagaaaagacccaagagagagcaaccctggaaagtggggttgctgtagaggaaaccctggggtggggcatttcctgtgaccctgagcggggaggaggcaggcgagggtcacaggagacaaaggcagccagccaatggaatgacgcatgcccattgctgagccctaggtcagatatgctgaaaggtaaagtacacacaaatccaccacgatcttctaaaagtgcagacctcagctcagtgggtgtggggcatgtgccaagactatgtttccagcagactcgcatgtgatgttgacgctgcagtccacggaccacactttgagtaacaaaggctttttttcttcttccccacagagggtgcctcagaagaggagatcagacttagcaagatggtgatgaaattctgggccaactttgctcgcaatgggtgaggctggtggcaaagacagagcacggctggtgagggtggggggcggggcatgcctattgggaaggggcagcttctaaggttctagtgatcaaacttctgaccctgtgaccatagcactctgataatgagagctctctgcaaacggagaggccgcccctggagatagtgagctctccatctctggaggtatacaagcctcttgacagagataacttgggcatcctcacacatctctgaagattgttgggaacacacagcagctttggggcaattctaattgattctgtttccagaaaccccaatggggaagggctgccccactggccagagtacaaccagaaggaagggtatctgcagattggtgccaacacccaggcggcccagaagctgaaggacaaagaagtagctttctggaccaacctctttgccaagaaggcagtggagaagccaccccagacagaacacatagagctgtgaatgaagatccagccggccttgggagcctggaggagcaaagactggggtcttttgcgaaagggattgcaggttcagaaggcatcttaccatggctggggaattgtctggtggtggggggcaggggacagaggccatgaaggagcaagttttgtatttgtgacctcagctttgggaataaaggatcttttgaaggcc

SEQ ID NO: 8 - DNA, 33573 bp, Homo sapiens
acagtccctctgagctgcacggagacctcgcaggcccccggaactgtcgcccttccaggatgtggctccctgctcttgtcctggccactctcgctgcttccgcggcttggggtgagtccttctgaagtcaaatatgcggggcactttttgaaatccttgttctgggccgaactgggcgcagatgcgtagaaaggcaaagacacaaaaggtccggctccgtggcggggcgcgacctccgtacttggaattggacttggagcagctgagcccagccagcctggcccgcgcggagacacagggagggtgaacatgctgagaccgccctgcactgcttgccccaagaaggccggcagccggcccacctcactccgcacacaagagaaatttaggcagctcagtttattgtcccataattcgtcccgttatggctcgataacggatagcggctcaggagaatattaagagcatcgactctggagccagagtgcctgggttcgagtccccgctgggcgcttcctcgctgtgtgtacttaggcaagagactgagctattctgtgcttgggttaccccctctctgaaacagaggtggcagtgctacctacctcactgggctttggtgaggaggaaatgggtggttgtttctaaagcactcttggaagcgcctggtatgtagaaagcatgaaaaaggggtggataaatttaaaggcgctgtttaaaaatgtagtgtatgaatagaaaattcgagctgtggtcaggccactatatcaggacctcttgccattaaaaagacagtttcttactcatgttctcaacaggagggggacaccagtgcgcggggcacaaggggaagcagcagagcctggggaggggacagggcggatctgaggccaaggggcttttactgtggcttccaaggatagaagaggaaaggcagggcaggcaggctgaggactggcaagttgggatacttcaggggctctggggcacaggggctgttcctaattgttttgtacctagccctggcatgattagggcaggtgggtagtggccagggcatggcaacccgatcaaagaggcggttggagtgtgggctccaggttggctggaccctgagagggagtttctccaaggtcaacaaggcctcagatgccagagcatcagaatacagaaaacaaaacacatggtacatagaggggtgcagcgaggggcctcgaaatacccccatgccattttatagacatagaaactgaaactcacacagggaaatgtacttgcccagggtcaacccgaaaatcacgcctaggtctgtctctgggtcctgaatcctgacagcccagccccgtctgcatcctgtcttccttctggcagcctgacactgcgagagctctggaactgtggggaagggcagaggaagagaggaagcctatttggaactgaatgttcctttacctctggttactcctggcaatgccatccatttccttccttaggaagaccatacaggctgcctccacttcctcagtttcctcctctgtaaaatgggcatgttgatgcctacccagcaaggcttatttgaagattaatttaaaaaactggaatgcatgcctggcacatagtaggtgcccaccatgttcgtgcttcatataacatcagcagcaactggtcttgttctcacacttgtttctgcctccccccattcacccactttttcacccttggcaatctggctgccatccacctcatcctgttgaagggaatccctgagctgagcatgaccagcccagtggtttcttctctgacttgactctcctccacctccctgaagtcctcagcaatcttagttggttccttgccctcttcctccctgggcttccaggacgtgggaacgttctctccctgctactctgatggatctttcgtcttggctgtagtggtccctgtgtttgatctttgtttttttctcttccctctcacactggcatcttcaatgaagccatacaaatccatggtgtgtatgctcactgtctaggagaacattctgccctgctcccccaccaggcttccttccagccttgaagtgcactcgtcctgtggcgtcatcttgccatgatatcccactgacatctccaccgagcctatgtaaacagggatccaacatcacaagccaggtctttgatcttctttcccttccatttccccccacgtctagtcagggacagagtctttttcttttcttttcttttctttcccttcctccctcgctcctgcttgcatgcctgcctgcctgccttccttccatccttccttccttccttccttccttccttccttccttccttccttccttccttccttccttctttcctttccttctttccttcttctctgtctccagaggagccaggattacaggctcatgctaccacgcccagctaatttttgtattttcagtagagaggggtttttaccatgttggccaggctggtcttgaactcctaacctcaaatgatctgcctgccttggccttccaaagtgctgggattacaggtgagccactgtgcctggtctccaagtctttcaaaataccttttagacatcatcatatttctacatatttttttctttttttatttaaaattttaaaatagttaagatcggttgggaagatttcaggtgaagaccagaaatcccatacagtgctgaaaaggtggaaatcaccaagaaaacaggccacatgtaatgccaccacccattgctatgtcgataaacatgcttccaaggaagttcatctcagtattgtttgtaattagcaacaacaaaagaaaagaagctaaatattgacattgcagtgttggctattaataagatgagacatggatgctgttgtgtaactgggctttgcataaggagtttccattgtgattggatattcccctctaagaccaaatctgaattgttctgttctgttctagccccacactttgagggaggtgttaacatcttgtctttatccaaagcagagcagctgggaggacaagaggctggaaatcatttcctaggagggaagctcagacaaactgaggtgactggagccagataggacctgaggggtgggcagcaagatcaagtgtctgaaaggccacaggctgcagatcagatcaggccagtatagtcccatgacaggtagaaccagaactgatgcggaggctgtgggaaatgaatttcagctcagtgaggaaacattgtctgtgctgaaagaatggagcagacctgcccagggccaccagaagaatcttccttgacctgatcacatcctttatctggaacggttcctgctgcttatttgtctgtaaagaaaactcaagcgcttagcctggcattctaagccctccctgacatgcgcggtctcattccaggaatattttccactccccccagactccacctggtgcatgtgggccatgcccccttccccaggcaggctctgcattctgctgctctgagcctcagcaaattcccttctctctgctgtgtaccactctctgcttctcatctcccctcattcaatcctcctctactgcaagtcccagctcctccatgatgcccgcaatcagaaggccttgccccctcttctaattcctagactgcgttatgggtacctctcccaggacacggccacttccttcccagctgtagccccaggtgtatatgtgacccttccctatgatggtcagtgcttgtgagaatatgggtcaccccttgttcatcctctcatccagcatgcggctcagtgccaggattctaatggataaatgtttccagagacttctaagggggaagctaaatgtcttgttctttcctagtagctatccttcttgcatttatttttggctggatgtttttttgcctccaattctaatttgctccttaaaccagcttgatgagttaggaaggacattgatccctcatccctattgtacatcaagagaaactgaggcctagagggtttaggtgacttatttaaggtcactcacttagagagtgggaaaccccacctggaatctgggtccagccttttgcctctgatgcatcctgatttcttctccatatccagcagggcatctgtcctcgccacctttggtggacaccctgcatggcaaagtgttggggaagttcgtcagcttagaaggatttgcacagcctgtggccgttttcctgggaatcccttttgccaagccgcctcttggacccctgaggtttactctaccacagcctgcagagccatggaactttgtgaagaatgccacctcgtaccctcctatgtaagccaggggtggctgtggcatgtgtccttggggatgttcgcctcacagtgatgcaggaaggagtcaaggcagtcccctgatgggctgatcctttgctctggaatccttaagatcattgtagatccttaagaacattccagaactctcacagcattctggagtccattatttaacacgtttattgagcacctactgtgtgccaggcatgttctgggttcttgggatccattagtgaacaaagcgaagatccctaggttcatggagcgtgcattctagcagctggacagataaaaacagcatgcaaattactccatgcgtcatgtgatatgttagaacggaataagtgccttggaaacaaagaagcagttgagcaggtgagggagttcaaaaggaaggatctcatgcattcctctgtcttgaactttcacatagttggggagagggctgggccaggaagtactgaggggtccacttggcaagtgctcaactctcaggtgtgccaattcctgaggagaaaaatggcaggtgtgcccattcaccctggccaatctgggaagaaaagcccaaaggttctaattggcctcatcaccactccctagggtaccaaggtccctacaggcatcagagcagaccctgctcatcttggcagccaggtgttcggaggctttccagcagcccgcatctggctgccttcttttctttgctattttttaaaaaataatgtttactgtttttattttctgattgcaaatgtaatatatacttattacagaaaattcaggaaatacagagaaggaaaagtaagaaaattaaaatcacatacaatatcccaccacccaaagagaacaactatcaatattttagatagcaaagttttatgatataatattttgtttgtattccagtttttttgcctgttgctatactctcttcctttaaaagggaatgctatattgagataatttggatgcctgctttgttcattataagtaatcatgagcatcattccatgccattaaatagcacagattggcattaacagctgctttacattgtattgaaccataattaatctccacaagcttttacttggggtcattaagtgacatatgatgttttcaataatataacatcatgaatgtgaagccttatgcacatatatgaatattttaggatagctgctcagaaacgacatttctgagtcaaagagtatatacctcttgtttgatttccctcccaaaatattccaactacactcctcccattgatgtatgaacatctccatttggcagactcttggcaatgcttgggattattaaaaaatgtttttggctattggataggcaaaataataataacaataacagcaatattattttcctctttaatttgcatttatctgaagactagtgaagttgagttcttttccctttcttcacattggaatgtctttttttttattattattgttgttatgcctcttgcacttggtgatctgtaggaaaatttcccaggttggaaatcaagaagtcatctttgctggctctgattctgtcacttgacagctgtgtcatcccagagtctgagtgcctcagtttcctcatgtacctagtgggggcggtgatgactgccttttggtacattgaggtctgggactgttgcttataatatacttgtgtataagcaccacatggcatggatcttcttggctatgtgatgttggtcaggttctctaacctctctaggcttcagttttctcatctgtaaaatggaaataatagaacctacctaatgggactgttctgaggcttaaatgagttaatatacaaaaggtacttaaaacagtgcctggaacactgggagaactcaataactgttaccaatgttcatctatgttgtatcctcaagatctttcatcagggcaaccacagcagcacttgcctgggataggcggatggtagaatcttggcatgtcagggctgcaagacccatgtagaaatcactgctctcacttagacctggcagatttgggacccagaatgtgaaaggaattcacccaaggtcatcccgtaaatgtgtgggagagacaaaactagagcacagttctcctgatgttgtcccaaaaagattttacccctaagacattgcaagaaatgtcacctcccagggaagatcgttgtattggttttcttccctccttgatgctgggagtcccgaaggctccaggaagggaagaggtgtgaagcccttctcactctgcttggtcttaggagaccttagtgagtcccagcacccccacctgaagccactgatagcctcctacccactacaatgtcgtgagtctgtaaatatcaagtccatttccaggcttgaacctcccagcccaaggcactgccctagaacacacctcgcttagatctggctgaacttcaggggttcttctttccccctccccaggttcacccaagatcccaaggcggggcagttaatctcagagctatttacaaactgaaaagagaacattcctctcaagctttctgaagactgtctttacctcaatatttacactcctgctgacttgaccaagaaaaacaggctgccggtaagccatgggatcccctggtcaaggcgtgtcagcgccagtaccctgcatcttctggggcagaatggaagggagtgaaggcagcttggggagggagctgcacaggccaggattgtttcagagaccccagggcctccacaggaaacactggttttccacacctcagtttcccctcatgacacagggactctgggggagtttgctgtgcatctttaatgaacatcttaattgttccaagttccctcatgctaatagaaggatgataatgattacttttagttactagattaaaaatctgatatccacagtgggtaagacatgattttcaataatgttagtggtggaaggaacttagagaaaagccaacaccccaactcttctttttcaaatagagaaactgaggcccagacagggaaggagaggtcacacaatagggtagttttggctcaagtcttctgatcttcaaatcagtgctttcgacagggattagggcattgagcttttatgtggccttctccagtacatcaacattcatctggggtaactttcactgtccaggtacaggtgttgagggaagaacatgggacaatcccagaagcatttccttttgcctttggccaaatatctgggattttttttacctaatttccctccttaattgtcttggcaacggctgatgacccagtgagttagcatttcccaaataaatgaagggcgggagatggggcaggctgccagctgcaggttggagggtggggagggtctgctgtcaaaggaggccactccagggtgtaaaaagaaggggtgttgtgtccagccagtctcccaggggcaccgtggcatggtgcctaggagtgggggagcaggtctgcaaacatgggactagaagttggccctacgtgtactttgtaagacaaaggaagtgggagaaacgtttactcattcattcattcattcattcattcattcttctattgtgtactcagtgccccatatacacagttagacccagggcttcagggaaaggaggaacatgaatccactccacgcagcccctgccttcaaagatcttgccatctgggctgtagagacatattctctcttcagcaggttattgcaggccagcccttgggctcagccttcacacaaagatgaatgcacactcagtccttccccaggaggcagccatggattagatgtgtgggagaaacaggggctctgggaacctggaggcaggagaacctctaggaatggccacaggttctagaactacacagtctaggtttaaatcctggttctgcctttgagtagctgggcatttcttaacttttctaagcctctgtttcctcaaaatgagagtcaccacatatggtcagtcaagggtgaccactaagcttgatctcggaccctggttaagtaggcagaggcaggctcatacccctgccctaacagtggactcactgcactgtggccggcgcctgcagtatgctgggcagctcaacatgtgaactctggagccagaccgcctgggttaaagtcctgctgctaccacttttgagttgtaggaccttgggcaggttaagtgcctccattttcccacctggagaatgccacagtattagtatctacccaatggatagtttattttactgtgcttagaacagcacttgggacatagtttatgttacgagttcttttatatttcatgaataatattattacaatatcattattttagccactctcacttcttccccactatgtggacttttaaaatgcaagaatcatgcatgaggaattgtatccactctcatcctccagtattagcagagtccctggcacttgctaagttctcgacagtgttagtccattgactgaaggcatcctgaagtgggtgggttttaggctgggttctgcagggtggataggatttggggatgcagaggcataaggaggcatttgaggaagagacaccatcacaggcacgtgtgcttctcgagacaatcattcatgcacagagaagtttacttcaccaagagatagttaactgagatgaagctgagatacaagggctgccccatgggtacgctgagtgcatgaatagtccaggcttgagggtgatgggagtgagggggagaagaggacatccttagcctttgctatgcccatgatgatgttctcagcatcaagagcctttcggggaggaggcactggaggctgggtagaaaggaagggacagccagggtatgtgcagtagggtggtggcttgggtcccagttgggagaattatgcaggagagagattgccttttgcaaagttgctgggagctggtgaactttgggtagcatcctccttcagttgtaaccaacaggtaggtcaccaaacaagacatcttctgagtgctccctggctctgtgtcctgtaactggtatgttgcttcctctctgcccaggtgatggtgtggatccacggaggggggctgatggtgggtgcggcatcaacctacgatgggctggcccttgctgcccatgaaaacgtggtggtggtgaccattcaatatcgcctgggcatctggggattcttcaggtaagaaattggactctcctcactgcactttggcccccagaacgaggatgctaggacccagctcttgtcatgccagccctcaggggagcttagctaggttccacagtaaggcatccaagccccttcataattggacactacctaccctctcactacccagccactcatccacttgcctctgagcttttgcatgtgctgttccctctgcctggaatgcttgttctatccgaagaacacctcttcatcctgcaagacccagcccccagttacctccactgaaagactcatctcctcttcacctatgtttctccagcacattgcttttctctgtcatgacacttagcaggcagcccagcaggtgaaggttgacacatctgacttctcatggaaaggagggaaggggcagagtcacagagggagctcagggcatgttgtgggtgaaggaatggggcctgcagatggtggggtgtgggagcatctgaccccttcctgtaggagtgttgggacccactcaccccattttctgctcgaaatttggcaagcgcgggaagcaaggatggagtcttccccatcaggtctccatggggacaggagtcagatctccttcaaggtgcaattactatgaaccttttctaagtgttaacatcccagcacctcaatcccgagtgagtgatgcgagggttgtgggagggattccactcactgatgagaaagcctagggagatggaggttgagttctggtctacccatcagcaaggcatcccaggccagtcttggtggtatcaccttcatccctgtcagccacagcctgtgtctctgctccccattacaataaagctgcttcatgatctctctccacttcctctcctccgtgtgtctcctccacccatgccagtcaggtttcttcctgaacacaccactaggctgctctcattgtggtcaccaatgacctccccctgtgctccacccaacctcacaggacttcccagcagcagcagatggccgctcagcctctgtgacatgcttccttcccagcccctttcctggtcccccctcctccctgtccacaggcctctcttgtctccctctccctccctactaattcccagtgatcccacggagcagcccagagcccctgtttccagtccacctcacctttgaagcccacaggtgtcactcactgcctcttccctgcccctggctgtttaaagggtgccccactcttcccatggacaagctgaatccttgactcccactgcccctccccaacagagaccccctcctcctctcaccacacctccactcagctgctgctgccacaccctcccttgggctcaggagaaggccttgtgggcagcctggactctctcttttctcacaaccctcttggctccaccttcagaatccacttagaatccactactttgcacctcttccctggctcctaccctagccaaagccttctgtctgtcaccttgattatgatcttaagacttactttcagatctctgccactttcttactgaggctgagtcgcacagtaccgagagtgatcacgtaacatgtgcgttgaatcatgtgatgcctctgcacaaaaccctgcagggacttcccatgttccttaccatggcagccgaagttcttgccagggccccagggctctctctagctgcacctcctgacctacactttctttcactgggccccaggcccattgcacttgctttttcttctgcctggaattcctttctttggatatttacaaggttcatgtgctcgttgtgcttgttttatgaaggtctttgcctccaaaacctcaaatgccactgcctccaaaaggccttccctgattgggatctgaaatggcaccccctgtcccacccctgtttttacccttgttccattttatttacctcaatgcactttgactacctgacatcattatattctctctatatatctgtttatctgcttgtgatctgtagtgggagctcagtgtcatcacaggttttgcctgttttgctctcaggtgtgctctagctcataggacagtgtctggcatatagtagatgctcagtaaatagttgccagttgagtgaatgtagaaccatacatcaccacaatgctgtactaatgaaagcagggtctcagagatgctgaagcccagccttattcttaaaggcttattgatagcccccagccgcatctgtagtcctgaaagtcctgccatgatatctctgctccccaccctcaacctgttctcttccttacagcacaggggatgaacacagcccggggaactggggtcacctggaccagctggctgccctgcactgggtccaggacaacattgccagctttggagggaacccaggctctgtgaccatctttggagggtcagtgggaggagaaagtgtctctgttcttgtgagtattcctggcaccgggcccaaccccaggcttgatgtgatgctgatgagatctcttggacacctgtcaatccagctatgcacacttctgattacagtctcaggactggccctactcactgcccaatcagggggtggaaagctaaagaccaagctctccttggcccacaggaagctcagagctcagcttagtgtccgggggccatatgaatctctagttgcagcctgcaacggtggcattctcctcttctagcttgattgagctctctctctctctctctttctctctctccttcccactcattttaacataaggactcacttcccttctcttgggaagtagggataggcataaattatgagtaagggcaggcgaagacaagaggtggacagaggtcattgtttagcttaaaccagaactatgagtggaggggacatggacactgagcaggctgggaattcctctggggtggtctcatggctggtccatgcccaagaaggaggcaacagtttctgtgactgtgggacccggtcggctaggggaggcgggtgagaggaaggagggatggagctggggatggggagggatggggcaggggttgtgggtcatagacacaacccggggtgtgaggggtcctcctggggttggggattgggtgcgggagacactgggggatctgggatgaaaacccagatgagaggtgccgggagctgtaggaagacttctgcctgcctcgaggtgggcagagggtcagcccactactggattcctcaatcctgtgttggttttatagtggggtagatctagcctggaagagcaagtgagtcactgaccccactcctgagcatgaactctcctcccctccactctgctgtcaggttttgtctccattggccaagaacctcttccaccgggccatttctgagagtggcgtggccctcacttctgttctggtgaagaaaggtgatgtcaagcccttggctgaggtaggtctccggctggtacgtctccggctggacacccccacctccttggctctatgctcctgaatcctcagggatctctcttgtggtcggttgtagctaatgttctcctagaatcactgaggcaccaatggctgagcaggaagggcgaggagacaccttgatcagcgtcccagtttcacagccaggcaaaccgacacagggcttggaagggatttgccaagggcagcaggtgatcagggcagagctgggactccagctcatggccctagcagccagtacagtgccctgtctgtgaccacactccacctatgtgccagggcctggtgccatattgggcagtgatggtgtcttgtgtctctcagggtctgagttctgtggacccacttgtgggctgtgggcctgaagcagttccacaatgagcgcctgataaccagggttggttcctggagaattcactcattgattcatttgttcacacaacaaaactaggtgactaagtgaaggcaaaaacaagaaatgggcagacgtcatcctttggctcaaagccagatgtccgtgtggaggggacatagacactgcatggccctatgtggctctgcattctagtcagacacctaacacctccccaagcttctgctataatgtagggaatagatgaatagctacagaatcacacaactagaaagtgtcacctatgacaagagcagtgaaggtgaggtacttgctgccacagcagaatctaaagaaagcattgagtcctggggctggaagaggttaccagggaaggcttgctggaaaagtgactaatgagtcaggagcaaggaacgtccagggagtgggagcagcacatgcgcgtgctgtggcaggaggacgcatcccaatcgggagggagagacagagacagagccgatggggccagagcaggcagaacaggcggagcgcggcgagtactgacagaggggacgttgggaggggccaccctgcacaggaccctggcaaggattttgtcatcatctggagagtggttgaaagccaaaggaagaggtgatcgataggaatccagacctaggctgaggatcgcccactggagccagtggcatggaggatttcggtagctttgaaagcttgtttggggaaagcatccaaatttaaagggctggtacataggaggagagaaaatggggatgccaagaatttttagaatttttgagaattttttaagaattcattggttataagcaacagttgcccattgaccagacttaagtcaagaaggagcattagcctggtgtggtggctcatgcctgtaatccctgcaatttgggagaccgaatgagaaggattgcttgagcccaggagtttgagaccagccagggcaacagagtgataccctgtctctacaaaaaaaaaaaaaattacgctggtcatggtggtgcaagcctgtgggcccatctacatggagtctgaagagggaggatcacccaagcccaggaggtctaggctgcagtgagctgtgtttgcatgacggcactccagcctgggcaacaaagcaagaccttgtctcaaaatcttgtttataaacggagaattaatttttcaaggaaatagaggtctgggatgaggctgggctttgtgaaaaaaacacgaaacagaaattacaaaatgatgggaatcctagactctcttattctttttcctctttctctcctctgtctctctctctgtctttctctctctcgccctctacttctccctgtttatttattcaagaaacaccctgggtattgattctgtcccagatcctacacgaggctcttggggcatgtgcactcgccctggctcccccaccatggagcctgcctgctctccctctgtgcccttatttctggctgcactttgcttccagtctgcatgagcaccctatcagcccctgagcttaaggactctcctttggttccagaaagtagtcaggctgtcttttctgttgtgaaaaatgagcgtttttacattagcagctctccaccctgattccaggtcacttaggagagaggctcactgtctcgtctgtgcttcagtgtccctggattcttagaaaagaaggaaaccagagtgtgcttggcagactgaccccaatccccaggacaggcagaaggcagcctctccctcaggagggctttttttattttaatattttttacattttttaaagttggaaagaggttcttggtgcattaattggtgttgctctgtggcttcacttaggacaaaggtgaacagaccatcactcatggccaaatctggatctgtgcctgcatggcccttgagctaaggatggttttttaaattttttgatgattgaaaggaatcaaaaggagaataatatttcatgaaccataaaattatatgaaattcaaatttcagtgtccataaaggaagttttctttgaacacagtgacactcattcatttacctcatgtctatggctgtgtttcatgtcaacagcagtgttgaagttacagcagagaccacgtggcccacacagcctccaatatttactatttggcctttgatgggcacattgccgacctgtgccctaaagagtcgtgcttgattgctgcttgggactctagtacacaaatgtgcaggtggaaactcctggccatgaaaccccatctatgtcttgcaaaggatgacaccctggtttttttcagaactcacctttaaatcacattcgctaccttctgaagcacctctgtgtgaatcatcttgtctaggcctcctacaattctgcacatgtttattgtgtgggagacatggcactctcccacatgtgttaactcatgaggtaggtgcagtcattattcccatccatgagtggagaaaccaaggcccaaagaaatcaggtggactgcctgcagcctaatggctggtaagtggaacaaccaggatttgaaagcagacaaggaaattcaggattccatgctctctatcccagctccaccttgcctttcatgacacattcagctggagatgattatgtccgtttcaatggagataaggtagcagagatgagagattacataattgacccatgttacaattgagattagtaacagaggcaacgatctgtgggacctggaacccacacccatgggtctacagtatcttctgctgccccctgccactggtacaagttgggcttgggatagaatgccactttccatgttgatggagggaagggacttcactcttgaactctgttgcctgtgatctttgcagcaaattgctatcactgctgggtgtaaaaccaccacctcagctgtgatggttcactgcctgcgacagaagacggaagaggagctcttggagacgacattgaaaatggtaggttgcctgttcccgtagcccaaacactgtaaacttggtcccagacttcttcatttcagcggtcctcttgccctgggacagttacctgggacaatttctcaagtctcgggagtctcagtatctgaatggggaatctaatttgtcctttttaatcgtaaaatggcacaaatgtataaaaagaaagtcaaaaaataacatgataaagaattgaccaaatttactatttgatgaaaatttaatattttgccattcttgttttcagatgtatttttgagaaactaaacattacatattcccaggggacaccatcatgctgaatttgggggatggagatattaagctcaaagattttcagaaagatgtcacaatttatcttggttgacttagaaactgtctgtattagacctggtggtggtccagttacatatattttctgagactctgactcatttattgttctaggatagtcatttgtccactcaatcattaatccatctactatgatcttcttatccatctgtatgttcactaattcatcgctttcacttatctatttttatcaatctaatatcaacctgttcgtaactttttatgtatctattttcaatccatccaccattcatggatcatccagccaccttatatctcaactccatcacccattcctccaaaatcaacaatccagttatcgcctgtctgctagttttcacccatctattcatgtatccattcaattcaactgtactccatgtattgaccaactccatccatccctccattgatccatccatccatccatgctaaatatgtaggggtgggtgttagaggtagcaaaacagacatgaagtggacatagtccctgctctcaaggaactatccaaagagaaatacattcatatacttcgcaggttgagtatggtggcagcacagaggggaagcattcattgtagtaatcagggatgcttcacagagaaggcggaggagccagatttgagaccagacagtggaattcaggagttcatgcccttaaccctgactccaccttatctttcctgagaaattcagctttcagatcattgtgcccattttaacagaggtaacaaagacagataaattttgtaattgccccatgtcacagttctaattagtcagaggcaacactgtgggacacagagcccacacctgcgggtctagagtatcttctgccttccctgctccctgccatcctcacatattaggacctggggacctttaggattgatggatggctgtggcagtctctcatcacagggaagcccagcaggacaaacacccagatgacacagcacccagggccgttgggaactattccctttgagggagaagagtgtgaattccaagctcaggagtagcctggactctctctttggacctgaggctactcctgggtcccagggccggcctctaccactggactctgcttgtgtttccatgggttgagtccaatgtggtcttggggcttaggtcttggttcagtctctaagtgaccaaagtgttcaaggaattaaaacacacctgttttctaatgccaggaaggaggcaaatattccagcaattctgcatgtggcatcagaggacccagcttaaaaggggagagttgagtctttgggaaaacctatccctaagatcctggaacatccttttgagtttttctgagatcttgtggaagtgcttccatgattacgcattgcctccagtatgcacacatgcagacacacagacaaacacacatagacacacacagagaaacacatacaggcacacacacacacacacacagacacacacagacacccacacacacaaagagacacacacacacactcttggctagtgggacttcaatccttaatggagggacatgcactggttacgtccggagcacaggtcagatcttggaactacaatgcaacactacagtcactgcaatcttggtgaacacacaccaaagagaagcatggggtagggtgagtctcagtgagcttgcatgagtcagggctttctgataggagatgaggaaactcccttaagtaccttgaacagaaaaagaaatgtgttgcaatagcatcctcatgtaaactgaaaatctcaggagttgatggcttcaggcatggctggatccagatgatcataggatattattgagaatctaccattctctatccttcagctttgcacatttctgtgttggtttcactcttaggtagatgtgtctcctctgaaggttaaagacaacagcactctggacttatgttctatctgcttaaaaaccctgtttctaaataattcctgcaaaagttttggggtaaactattggattgacatgggtgtatgcccatccctgaactaatattcatgttcagggagatggagcaccccaggtcacatatagatatatgaattcatggagtgatgggagaagaaggaggtctctgaaggagaattggtgagttcttcccaggtgaagaggaaggggagagggatggggggccaggcagcctcaaccagcgggggtcctctccagagctccatcgaccctggctcagagtccccggcctgtcttccccatgctgatgcccacccctccctgctatgccttcaccctgtgcttaggcctagcagagccagagtcctgcccacttcctttcagcccagccagcttggcaccaggagggagaacctgacacccctgttgccccactcacccagctcagtgttctcctgggaagcctctcacccacatcctctgcttttgtcttcacagaaattcttatctctggacttacagggagacctcaaagaggtaaggaccttttgtttctggattacgggttttgagtcttagcacctttaagttccaatgaactgtgagtgaaagaatcccctctcgggtggttatagtaactcctgtgtgtttgttgccgaggcccagagaggggtagtgactcacctgggtaacacaaccaggaagactagtggctggcctggaacctattttcctgactcccagtccagtgctcccaggcatcacctctgtgtgccctgggctctgcccactcccctgcttttttacattttctactccccaaaatggcactatggaagaaggctgaaccagaatcttccactctcaggaggcagagataatagcgatgattagccatggagggaggaagcctaaatctcagtccaaggacactcgcctcctcccagcacacaggaaactccaacagtatcctcctgatctcctcacccccacccccacctcccaatgggttgacagtttctggtgacatcacctctgacgaatcttacaatcctgtcctctctgctgcctccctggaggccgcagcactctcctaaatggtcatgggcggagtacatgagattcattcagcaaagataataaacacttcctatgtgccaggacttgtttaggagctggggatataacagtgaaaaaagaaagactccctgccctcttgggaccttcctttacagtgcaaggacagtgcaataaaaaagcaaatcaagtatgtattccatttgatggtgtgcagcattaggggatagagtttgtgggtggtttcaatttcagtagcaggatcagggaatggttcaatgagaaggtggcatttgagccaagctctggaggaggcaggaagccagctgtcaggaaacctggagaagcctattccaggcagagggaatagtcactgcaaagaccctgacagaaggggccagtctggctggagaggaccgagtgtggggacagactggcttgaggctaaagaggtaattggcagtgggagagagatcaattaaagtacttggatttggattcagagcaagatgggaaacctttggaggttttgaacagaggagtcacatgttcttagatctcacaaaggtctctgcctccggtgttcaaatagactgtaggaaaggggcagagtggatgcagttgaccagctgggcagccactgcattgccgtaatccaggcaagggcaagggctcctggctgcttagacagggctgtggcagtggggatgggaggagtagttggagtctggatatctttgaaggaatagctgacaggatttgctgatggacaggatgcagcgggtgagaaagagagaggagtggaggacgccctgagttttctgacacaagcaaggttatgcccacacagggcagccacttgcaaactctgagtggacaacagcaatcacttgtcattgttgctgatggcagtcttcctgcattgctgctgcccaactggggctctcctgtctcttgcctcaacttttatcctcttctacttcacctcccctccccatttccacgtctcatggtgcagccagactgtccttccttaacagagttagagcacgtcactcccacttcttaaaagcctgaccttccttatgagacaaatccccttccctccatggagttagtccttcaaaacacacctgcgtgcacactgtgtgccagactgtgcagaaggtaggctctgcacctgtccctccctctagaccctgccccacagcatttctccaagcccagaactcttggttgtttctggaatttaccctgcacagtgacacctatgtccttttgctcatgcggttcccactgtttagaaattctcactcctttcctcacctgtcaataccattcactgcccagagcccaccgcaggcgggcggcacctcctccacgtagtctgccctgcatcttccttccagaggggctgctcctctttccggcccctcacggcagccctgcctgaactgcacagcctctcaaggcacccagacttgcaccccaatcctgttctgtgcgtcagccatgttggattaggatctgatcttgtccagctaggtctggtccacttttcagaacatctcccccaaggcagtccgaactagcttgtcattcattggcttttgtaggcagaccaaaggtcccggaagccatagggtctcaaaggctaggaattttccagttccatctgatatccaggagggaaacacagcccatggggcgtgaaactgggaagatggcacagaggactggtgtctatgagagggacccacagcaaccttgctaacaagtaatacctaacagttattcagcctttccagacaccaggtgctgtgctaagtattttacatgtgttcagctatttaatcctcacaataactcacctcttagatgagaaaactgaggcccaaacaggtgaaactcctagccagtaagtagcagagccaggcttcagatgcagttaatctggtttcaaagtccatgctctatcttagcaactacaccattgtgactaaatatgaaatataactgtacccagaaccagctgccctgatggcaacgcgtgagtcagactctctctaatctgtgaccacataaaaattattcattaaaggtaaaacctaaaattaagacatggatcaatatactgtgagttaatcactgattcttttactcataattttctctctaatagggaatctcgtcctagtctaggctccttgagtgatgagggttcagtacttcctcaaagccacccatggatcaataacagctcttgtttaaatacagatagatagcataatctcttcctcattaatcatggattttgcgtttataaattcacctgcttgctaaaatgtccttataaaccccaaatcgatactggtggcactttcatggtcacacaggcaggcacagagcaggggaagctggatttgcatgatgggcatgttccatctgagatctggtgaggcaacccctgtcttcttgtttcatctctgatgtttacaagtgtcctcttcgcagtctatttagtgccgtgtgttctgcatttttgtgctttttgtagtgatgtcactgtttagagcgaacccaagagtagcgctgctgtctggcgttcctgagctaaacaggctgtgctgtgccttacggggaaggtgcctgtgtgagacaagcttggatgaggcaggagctgcagtgctgctggccgtgcatttggtgttgaaaaatccacataatagcacattcagaaaaaggagaaggaaattggctgattcatacatgaggctgtgatagaaagggctaaagtaacacctattgtgtgggatggagccgtggaagtctctttcaacagaaacccacacataaaagaaggttgatgaaaatgttgtggccagaggtttgcaaaaacctaacccagtatttcccccttgagtgatgaccctgtattctctaattcagtactcatgggggctttatcagaagcaactgccaggactaccgaaaatcaactgtggacacacagaggaaaatgcatgcacttcaaattgaaatatacagacatgtaattttacatagataatacatgtatttcaatatgagtgaagatttcttcctctcctcatcatctgaccagtgtcagcctctgcctgaagttctacccctgagtcacagaggcctacccccttaagttaggaccctgagtatgtgcagccgtggcccatggccatgccggtctgtggtactggtctcaccctcagacaggcagagttggggacatgggtgtgactagggagggaatcacaggcactgcacttctctctcccccagagtcaccactatttggccaccgtgattgatggggtggtgctgctgaaaacacctgaagagcttcaagctgaaaggaagttccacactgtcccctacatggtcggaattaacaagcaggagtttggctggatgcttccaatggtgagaaggtgcaagcctcttggagggactcacccaccaccatcagccctcctgtctctagtcccagaagtgcttccctctccgagccacactctgagtcctgggcagctgttcccttcagcaggagttaccatttccatggaaatctgctccaggagggcacagttgtcaccgggatatcagtgccctgggatcccctctccccagactctctgattatcttcctgttgaagaatcctgaagtgtctgtgctgggaggaccagtagagaaaataactgtgcagatgggaaaactgagggggcctgcatgggggaaggagttgaggagtctggagaggcagtcgggggcttcccggcttcctgcctcgggctgggtgtgcgtagttcccctgaaaatcactcatgctcttcatcacctcattgaaaatatccaaagaaagtggcttcttcttgagttaccattgacttctagggaacagtagacatcagggtctactaagagagtggagggtcagaggaagatgaggatggaaaagctacctaatcgggtgctatgctcaatacctggtaacaaaataatctatacagcaaactccagagacccaaattacccatgatcaaacctgcacagttacccccaaacctaaaataaaagttggaaacagataaatggaaaaagtaattgagttctagaagtaaagaagcgttctccttccagttgaagataaacaaaactgacccttgttaaagcagtaggacaggtttattcagtaatatgattgcaataggggaaatattccggcgtggactggactcacatccccaaaacaaaagcctggtgatgtttcataagccaggggcgccaagggaaacacactgtggtctgtggggagaggcttggtccatgtgactagaccacctggatctcttcatcctggctcacctggggcagaaacaaactcctcttatctttaggacaggaagccatgcgttagactgcaggaagaatccactgaagtcaggctctattctttccacagggactgggaagatcaagagtgagctccttgaatgtttgcactgcaaagagaagggtctcacgtcccccaggaaacagggttgggtgatagattcacatcccaaacgacagagaaagggtttattattgcaggctttctaaagtaactgctctatttgggtccaggggacatctgcttttctctaggttttcgctggagcaagagtaaattatcctggcagagttgagcctccacaggcaggcatgttaaggatgctgcggtcattctagggacacagccttaagctgctagaagtgaggctagagtttgattgagtctgtaagtgcagaggttctggaagagttgttatgtgcgtagagatgtgcagttcacccctcagaaggatttcagaggttataccaaagaccaaagccatgaaggcaggatctaaggctttctgtgtcatctgtgccaggtgggggtacttgggaggtctgtgtcctcttccccagagccctctattctacactggcctggcagaaactttccctctgtgtgtcactgaccactgtagggatctcaaccacacacacatacacacacacacacacacactgcaaaacactgtcacagcgtctccaattttttttttataatctttgtaatgcttgaatttctggatgtaagcccgtttgattcattaattttgccaatttcagtaaaaacccatgcccctgaaagcccaacaaaatttgaggaccttcagactgtattgatttggttgggtggtaagtttgtttcttctcattaattcccaatgactcataaacgcttaacttttttttttaaaacagttgatcagctatccactctccaaagggaaactggaccagaagacagccatgtcactcttattgaagtcctatccccttcttgtaagaatctaggaatcacgggaattggctgagacctagagagggacaaggacttgcccaaatcatagagaaattaaatggcagaatgaagactggggcctgagggtttcccccatctttccacctttcccacttcactttccaccctagcggggagttgcacagggcttgtgagtttcaccactgaggtaggccttcctggtgggctggagaagctacatcactcaccagggggtggtggtcactttattgatctattttagcacattcctaaggaactgattccagaagccattgagaagtacttaggaggaacagatgaccctgtcaaaaagaaagacctgttcctggacttaatgggggatgtgatcttgggtgtcccatctgtgattctggcccagaaccacagaggtaagtcccagaggtcgaacaggggagggacatgaaccccacctgcttctgtatctgacccacttacctcccaacatagacagacatggaaacagctgagggtcaggcctcaaaagctgattccatatggcatgggatgaggtgctgtcttattttggtttatgccagcagaagactgtgagaaaaagaatccagaggcaagtggttttttttaaggtgatgatcccagataacagcagtagggagatggggaagtgagagaggaaagagatggaatccaatccatgatgtgttgccaagggagtatcctctgtggacaactggagctgggaaactcatggaaacacggagaatacagcactcatgggcatcccatctgagtggtaagggagccgggtatttatccaccagcttccactggtcattgtttgaaggttcttgcaaggagttgtttattccctatgactttgtcctgctgcacacaagggcagagtagtctcttgtgaacagacaaaggcctcaggctgggagctgcagatgctggaggtggaagtcaggctggcatgcccaggaagaggggatataggtgagacacagaaagcatctgctgcaagggctccatgcctggagttatcatgggacatagggctgctgtaggacaaatattgaaagatgagtcttggaaagtttttaaggtccaagctggctgcaaacaagtttgttaagtattggagctggaggaacagtgggagccctgcatgggatccagagggttgtctaggaccaagaagaccaaaaggacaagccagggcaggtgtgcgggaggtggtctggaaaatggcaatgtctgagcctggcttttgtctatctcagtgtgtgcaggaacgtgcaagcacagggccaggacagggtctatgcaggaccttctaccactgacccaaggctgtgtgggtggggcgtgaggttagggatgcttgggctcacctctagagagaagtatcctttcctgggtgccaactagaggaggctcacaggactctttcttccagctctcattcgcctagtctgcaagccaggaaatgtcctctctctaaccatgctggaagggtttttaaatttagaacttcataagcatgataaaaactagctaacactgatctagcaggttcccagacactcctttgcacaggaaggggcaggtgctcataaccctgatttcttcacagggtcttgataaaaccatttaaccaagaccactggagtttaatctgttctttcttaaacttggccttgcccaatgctctacgtctgatctacatatagtgttcccgtctccatcttctgctggtttgagcaatactgaacctctgttcttcatctgatttcccatgactttaagttcagggttttttttgttgttggtttgtttgtttgtttgtttgtttgtttttaccaggactacacagattttatcaactttcacttattgttgtctcactctttttgatgaaaataacatttctgaatataaaattaatatgttaaattatagaaagtttcaaaaatatggaaacacaaaaagaagaaactaggtatgactcataattccactacctggaggcaagtgctataaattttttgtgttttcatctaaagattttccatgtcctatatagtttaaaaatcaattttatattttgcttttgttcgtagaacactatgttgtgaggattttctcaatgttgatacaaactcatcaaagcatgccttattccatgaaatggctgtgttaaatagactttaatattcctatcctattagacatttagcctggctgctcacattctgatgctacaataacattgagctcagcctaatgttcataagtctctaaccacatgtctaataattttcttaggattatttttagaagtgaataactaggcaggaccacagaaacgttttagaactctgagcaaatattgcaaaattgcattccattgcataagaaagtggatttcttctttttttttttttttgggggggggcggatggagtctcgctctgtcacccaggctgagagcagtggcatggtctcggctcactaccagctctgcctcctgggttcacaccattctcctgcctcagcctcctgagtagctgcctgccaccacgctcggttaattttttgtatttttagtacagacggggtttctccgtgttacccaggatggtctcgatctctggacctcgtgatccgcccatctcagcctcccaaagtgctgggattacaggcgtgaaccaccgcacccagccaataatgtggattctttttctcatttgggatgctaaggttaaaaattaacataacgctgccagtttcataaaccttccccaccaataagaagcacatcagctagcataacactgccagcttcataagccttccccaccaacaagaaacacatcagcctgttttaatctgcatgtcctccatgactagccctctcattgtctctgcttctcttattatgtctaattgtcattacccaaatctgtgcgctactttcctctgaagacttgtgttgtgattgtaagaattgtataaatatcaatggctttagccaaagagttttacaaagtacgttttgggcagcatgctgcctataataagttctatcattcacaaaagctctttcttctatggttggctctgagtttctttggaaaatgttcgaggggcaaaggcaaggtcaaaccctccttttagcaagttttgtttgaccttgaagcagccatttaacaggtggattacagagccacagaaggatggcatcttcccaggtgcgcttcctgctgagccgagggtgcagtgcaggaggctgacaaagatgagtgaatagattattcttcattctcacattgaaatgacatgtgcgggtggggcatgaggttagggatgcgtgagctcccattgttaaatccttttgtgaacgaatttttaatgatgttaagaaatgatggctggaggcggtggctcttgccaacactctcagcattttgggaggctgaggtgggaggattatgagagcctaggatttcaagatcagcctgggcaacatagtgagaccctgtctctacaaaacaatttaaaagggaagtgattacagtgtactacaaaataccatatataatatcatccccagttaaatacataaaagagagatatgctaaaataaaaaagaagcattctctgagtggtagagttatgagtgatactttaaatttgattgttaaaattgatttttaaattatataggatatggaaaaattttagaaggaaatacaaaaaatgcatggcccttgcctcaggtagtggaattatgagtgatatctagttttgctttatgcgtttccatatttttgtttcttctaattattcctaattgtttttattttccaaatttcttactgtacccaaaggggaggacattgtaaattttctccaccagcctggcattgccctgactctttccgtcttagtgtcacttctcagtgggtatcatgaccagtgcaaagtgctgaggcacagggccaggcggcccacgaggtagccctgaaggggcagcgatctgtggctccaatgagccaatcattgggaaactggcatcttcctggggctgggacaggagagctttgttggtgggagaggagggcagagccgaaggagagagggcaggggagaggcaaggtcacccttgctcagcgccttcttttctctgccagggctttactgccttaacaccctaacactcccaggccaggcaggcagcaagacacatggggacagtgagctataaagcagtgagtgccgcaggttcctgagacacaagaggcagattgttccctctctgcatatgtttgtatttaaatagcctcatgttcagacaagcaacttggatttgcttcagtcttctcaggatgggtgagaaaaacactggaaatgctcaaaacgaggcagaacagaagacacctgtcatggtggagatggcatccaagtgaatgaacgcgagtctgttatcaatgaattatatccggtgtaaccagatgtagagagattgtcttgtgtgagaatgcaccagcaggcaaaggggtttacgaggcagagtcagggtccctgcaccggcagctgttttaaatgaattgacccagggtttccacattcattcattaaacatttgttcattgaacaaatatttggctctaattccgcttccacctcagacagctatgcagcccggagcacatcactgagctttcctgagactctgtttctctactgtgctgacagccagaggtttgctgcagagctggagaccttggtagcctccaggtgtgtgtctggcaacatgaactgtgcatgaatccttgtcttctcagatgctggagcacccacctacatgtatgagtttcagtaccgtccaagcttctcatcagacatgaaacccaagacggtgataggagaccacggggatgagctcttctccgtctttggggccccatttttaaaaggtaatgctccttcctgtctgtgagctaggagttagagacttgggttccagtcttggtgtggtggccttcagcaattttgtcctctcctggtcttagtttcctgattctgtataacagggttgaatgtcactgggttgtccgccggcttcctcccactctaacacatgtgaggagggatgtgtattcatgtgccctctgcacagacgctcagaatcctgctgtgtgacatcaagacaggtggtggtgggggtctcctgcaggattcactatgacagggttgaatgtcactgggttgttccccggcttcctcccgctctaacacacctgagggaggatgtgtatcatgtaccctctgccacagacactcagaatcctgggctgtgtcatcaacaagataggcggtgggacacatgcggggccctgctctaggttgagaagccaccaatcaatttccctctcccaggagcgctgagacagtgagtgagggtgagattagaattggtgttaaccctaagtcaggtctaatgtgtgcctgtttgtctttctgtctgtctctaacaaaaactcataaacctttacatttagtacatttaataattgttgatttagaattcacaggatgtatgaatttggaacaggagaagagaatattttttatatggtttatagtcagcaaggtggccatcccacaggctgggaaggtgcctctggccaagcccagaaacgggctcattggaggaggggttgggtaggagctttatgtgaacggattggctaaacatacatgttcaacaggttacagggggagcaatggatattcatgaagacagtcctgacacatgtgtattaaacaaacacgtatgtaacatggcccatgttcacctggtggtggagacctaatatttaaatgtattacaattagggcctgtaagtccaaaggtcttctcaggacacgaagctcagcaagtgaggagcttctgtacaccggccaggtccagtccatggctggtgatgttcttatctggagaaagttactgaaatcagtctcttatgcaatcaaagcagtagttaagtctggcaagtcagggttctgtttgtctgcgtatcccagctgcagccgttgttattgttttccttttgcttagctccaggccagtgctggtttagctgctagagaaagagaagcacctcgtggcagtgagaacaaagtggattcttttttttttttattattatactttaagttttagggtacatgtgcacattgtgcaggttagttacatatgtatacatgtgccgtgctggtgcgctgcacccactaactcgtcatctagcattaggtatatctcccagtgctatccctcccccctccccccaccccaccacagtccccagagtgtgatattccccttcctgtgtccatgtgatctcattgttcaattcccacctatgagtgagaatatgcggtgtttggttttttgttcttgcgatagtttactgagaatgatggtttccaatttcatccatgtccctacaaaggacatgaactcatcattttttatggctgcatagtattccacggtgtatatgtgccacattttcttaatccagtctatcattgttggacatttgggttggttccaagtctttgctattgtgaataatgccgcaataaacatacgtgtgcatgtgtctttatagcagcatgatttatagtcatttgggtatatacccagtaatgggatggctgggtcaaatggtatttctagttctagatccctgaggaatcgccacactgacttccacaatggttgaactagtttacagtcccaccaacagtgtaaaagtgttcctatttctccacatcctctccagcacctgttgtttcctgactttttagagatgcaggcctgaacccttgcccgacgtggccttaggtctcgtttttaatttggtgtcttattgccacaaagagtgtgttctgtcagaatgatgaccttcattttattgctgatgctggtccggtggtgtctaaatcacaaaagggagggagtattatgaggcgtgtctgacctcctgtccgttcccaggcaggaacagagttgaaggtttttcgaggctccccttagcccagagagggtctgttcagtcagttgggggtgtcaggattttatttttagtttacattatacaatagtttaaaaaatcctttcacggtgtcttctaagtcttaactctcagcagttacagatcagatgggactcaccccatggctgctcagaacgcaccagcgcctctgggcatctcactgtgcatgcttaggcgccttgcggctctgttgtttttcagaatgtgaattgtgggtgtgtgctggggagggagaaaagacccaagagagagcaaccctggaaagtggggttgctgtagaggaaaccctggggtggggcatttcctgtgaccctgagcggggaggaggcaggcgagggtcacaggagacaaaggcagccagccaatggaatgacgcatgcccattgctgagccctaggtcagatatgctgaaaggtaaagtacacacaaatccaccacgatcttctaaaagtgcagacctcagctcagtgggtgtggggcatgtgccaagactatgtttccagcagactcgcatgtgatgttgacgctgcagtccacggaccacactttgagtaacaaaggctttttttcttcttccccacagagggtgcctcagaagaggagatcagacttagcaagatggtgatgaaattctgggccaactttgctcgcaatgggtgaggctggtggcaaagacagagcacggctggtgagggtggggggcggggcatgcctattgggaaggggcagcttctaaggttctagtgatcaaacttctgaccctgtgaccatagcactctgataatgagagctctctgcaaacggagaggccgcccctggagatagtgagctctccatctctggaggtatacaagcctcttgacagagataacttgggcatcctcacacatctctgaagattgttgggaacacacagcagctttggggcaattctaattgattctgtttccagaaaccccaatggggaagggctgccccactggccagagtacaaccagaaggaagggtatctgcagattggtgccaacacccaggcggcccagaagctgaaggacaaagaagtagctttctggaccaacctctttgccaagaaggcagtggagaagccaccccagacagaacacatagagctgtgaatgaagatccagccggccttgggagcctggaggagcaaagactggggtcttttgcgaaagggattgcaggttcagaaggcatcttaccatggctggggaattgtctggtggtggggggcaggggacagaggccatgaaggagcaagttttgtatttgtgacctcagctttgggaataaaggatcttttgaaggcc

SEQ ID NO: 9 - DNA, 1393 bp, Homo sapiens
gcttcatccacgttgtagcatgttcttcttatggctggatgatatgcctctgcacgcatacaccacacttcatttacacattcatcaggtcatggacatgcgggttgtgcccactttttgactaggatgaatgacactgctctttgtgtacaagcttttgtgtggacatgctttcagttctcttcggtagacacctaggaggggaattgctgggtcatatggaaactctgtgttgaactttttgaggaactgcctacaccgcacaacagtttataacctattattttctcctttcacaacgcatcctgtcttcattgttaataaatagctgtgcacatcactattttgagtgaatacatcgtatttccttagccagcggtaccacagtgtgtttagtgaattccctattgttggacatttaataatcccatgaaagaggctttgagtgacttccttgtgtccccagcagcttgtaaatgacagttttccaggtgtgcccagagcactctgtatccccttaatttggtgatttcacattgccttgacatcacccctactgctcctccaccccatctgtcttgtctacaatggtgagccccttgagaagctgaaccatgtcttgaattggtctttgtatccattatacatggccaggtgccaagcattttggtaaatgttcacaaaatggatgggtacttggatggatggatgataacagccagggactatggggggacggagttcatttttacattctgtccaaaaatccctgctttggaaagtagtgagttctctgacaccgatggtgtgtgacaggaggctggaccagttggcaggatattgtgaagctaattcaggcctcagaaaggggactcgatgaaatttaagatcgcttccaagcttgagagcctggaaagctatgaaaacacaagccctgggagctgagatatgtcctaacttacccagctgagctgtgaggtgtgagtggctctaacattttccagttgtttctgaggacctcagatcaaagcttccctttgcctaaaagcatctgcctgctggtgtgggcccttgggggccgtcacagtgcactgaggttagagtcctgcaagggtgaacccttatgtaacaagtagttgggcaagtttacagctctctgtaatctgacagtagagtccagactggtttgatgaaagagggtaaactgtgggtgggcgtggcctgaggccccactagaagcccagggagatctgaggaaagggagggcttttctgatctctcccaattagaggattaggcaattggcagcgcagggcggtaactctgggcggggctgggctccagggctggacagcacagtccctctgaactgcacagagacctcgcaggcccc

SEQ ID NO: 10 - DNA, 1388 bp, Homo sapiens
ttcttatggctggatgatatgcctctgcacgaatacaccacactttatttacacattcatcaggtcatggacatgtgggttgtgcccactttttgactaggatgaatgacactgctctttgtgtacaagttttttatggacatgctttcagttctgttcggtagacacctaggaggggaattgctgggtcatatggaaactctatgttgaatttttggaggaactgcctacaccgcacaacagtttataacctgttattttttcctttcacaacgcatcctgtcttcattgttaataagtagctgtgcacatcactattttgagtgaatacatcatatttccttagccagcggtatcacagtgtgtttagtgaattccctattgttggacatttaataatcccatgaaagaggtttgagtgacttccttgtgtccccagcagcttgtaaatgacagttttccaggtgtgcccagagcactctgtatccccttaatttggtgatttcacattgccttgacatcacacctactgctcctccaccctatatgtgttgtctacaatggtgaaccccttgagaagctgaaccatgtcttgaattgacctttgtatccattatacatggccaggtgcctggcattttggtaaatgttcacagaatggatgggtacatgaatggatggatgattaaagccaggagctattgagagatggaatcatttttacattctgtccaaaaattcctgctttggaaggtagtgatttacctgagattggaagtgtgtgagaaggcttggctagttggcaggatactgtgaagctaattcaggcatcagaaagtggactggataaaatttaggatcgcttccaagcttgaaatcctggaacgctatgaaacacaagccctgggagctgagatatgtcctaacttacccagctgagctgtgaggtgtgagtggctctaacattttccagttgtttctgaggacctcagatcaaagcttccctttgcctaaaagcatctgcctgctggtgcgggcctttgggggccctcagagagcactaaggttagagtcctgcaagggtgacaccgttatgccacaagcagttgggcgagtttacagctctctgtaatctgagagtagagtccagattggtttgatgaaagagggtaaactgtgagtgggcgtggcttgaggccccactggaagcccagggagatctggggaaagggagggcttttctgatctctcccaattagaggattaggcaattggcagtgcagggcggtaactgggggccagggtggcgccagggctggacagcacagtccctctgagctgcacggagacctcgcaggcccccggaactgtcgcccttccaggatgtg

(19) United States
(12) Patent Application Publication
(10) Pub. No.: US 2014/0162896 A1
(43) Pub. Date: Jun. 12, 2014

(54) COMPOSITIONS AND METHODS FOR GENOTYPING CES1 GENETIC VARIANTS AND USE THEREOF

(71) Applicants: John S. Markowitz, Gainesville, FL (US); Haojie Zhu, Gainesville, FL (US)

(72) Inventors: John S. Markowitz, Gainesville, FL (US); Haojie Zhu, Gainesville, FL (US)

(73) Assignee: UNIVERSITY OF FLORIDA RESEARCH FOUNDATION, GAINESVILLE, FL (US)

(21) Appl. No.: 14/113,162

(22) PCT Filed: Apr. 19, 2012
(86) PCT No.: PCT/US12/34255

§ 371 Date: Feb. 4, 2014

Related U.S. Application Data
(60) Provisional application No. 61/477,475, filed on Apr. 20, 2011.
(60) Provisional application No. 61/491,668, filed on May 31, 2011.

(51) Int. Cl. C12Q 1/68

(52) U.S. Cl. CPC C12Q 1/6827
USPC 506/9; 435/6.11; 536/24.31; 506/16

(57) ABSTRACT

The invention features compositions and methods that are useful for genotyping CES1 isoforms (CES1A1, CES1A2, CES1A3). The ability to specifically genotype one or more CES1 isoforms (e.g. CES1A1) is useful for assessing drug metabolism in a subject and guiding treatment selection.

### CROSS-REFERENCE TO RELATED APPLICATION

This application claims the benefit of the following U.S. Provisional Application No. 61/477,475, filed Apr. 20, 2011, and 61/491,668, filed May 31, 2012, the entire contents of which are incorporated herein by reference.

### BACKGROUND OF THE INVENTION

Mammalian carboxylesterases (CESs) have prominent roles in the hydrolysis of numerous and diverse compounds including carboxylic acid esters, carbamates, thioesters, and amide containing agents. These substrates are represented in almost every drug class, as well as in many prodrugs and agents purposely formulated as esters for the purpose of improving oral bioavailability of the active moiety. Human carboxylesterase 1 (hCES1) is the major carboxylesterase expressed in the liver, and contributes approximately 80% of total hepatic hydrolytic activity and its functionality is important for the inactivation of numerous xenobiotics including therapeutic agents, such as methylphenidate (MPH; Ritalin®) which serve as substrates. Further, functional hCES1 is important for the bioactivation of a number of specific prodrugs, such as oseltamivir, and trandolapril. Finally, hCES1 is recognized as the hepatic enzyme catalyzing transesterification reactions between xenobiotics and some endogenous compounds with orally ingested ethanol.

hCES1 is encoded in humans by the CES1 gene. The expression of CES1 and catalytic activity of hCES1 exhibit substantial interindividual variability. Three isoforms of the CES1 gene have been identified, i.e. CES1A1, CES1A2, and CES1A3. Although the DNA sequences of the three genes have high similarity, CES1A1 and CES1A2 are functional whereas CES1A3 is not. Genetic variation is one of the major contributing factors of varied hCES1 function in humans.

In the liver, the majority of hCES1 is the product of the CES1A1 gene because transcription of the CES1A2 gene is substantially lower than that of the CES1A1. Thus, CES1A1 variants influence CES1 enzyme activity more than CES1A2 variants. However, current Taqman®-based methods are incapable of distinguishing variants in CES1 genes due to the high similarity of CES1 DNA sequences. For example, homozygous variants of either CES1A1 or CES1A2 are often mistakenly read as heterozygous mutations in a Taqman® CES1 assay.

The ability to genotype CES1 variants has the potential to improve a variety of pharmacotherapeutic treatments directed at curing or ameliorating symptoms of selected diseases and disorders. Despite rapid progress in methods for detecting CES1 genetic variants, current methods are unable to distinguish between CES1 variants encoded by CES1A1, CES1A2, and CES1A3 and/or to distinguish CES1 homozygotes from heterozygotes, (e.g., between the presence of two or one CES1 variant alleles). Thus, there is an urgent need to develop a discriminative CES1A1 genotyping assay in order to better guide pharmacogenetic-based therapy.

### SUMMARY OF THE INVENTION

As described below, the present invention features compositions and methods for genotyping CES1 genetic variants, which is useful for predicting whether a subject will respond to a drug treatment, identifying a subject as responsive to drug treatment, identifying the cause of exaggerated pharmacodynamics responses or overt toxicity related to specific drugs, or selecting an appropriate therapy for a subject. The present invention further provides compositions and methods to personalize a therapy and/or avoid adverse consequences of altered metabolism of a therapeutic agent or compound (e.g., enalapril, methylphenidate), associated with the presence of a CES1A1 variant allele linked to reduced drug metabolism in a subject.

In one aspect, the invention provides a method of genotyping a subject for CES1, the method involving amplifying a CES1 nucleic acid in a biological sample from the subject by long range polymerase chain reaction (PCR); and detecting a variant allele of CES1, thereby genotyping the subject for CES1.

In another aspect, the invention provides a method of genotyping a subject for CES1, the method comprising amplifying a CES1 nucleic acid in a biological sample from the subject by long range polymerase chain reaction (PCR); and detecting a variant allele of CES1, thereby genotyping the subject for CES1, wherein the alteration is in a residue that stabilizes substrate-enzyme intermediates or that alters enzyme activity; and wherein analyzing comprises nucleic acid sequencing, allele-specific hybridization, allele-specific PCR, oligonucleotide microarray analysis, or mass spectrometry.

In another aspect, the invention provides a method of selecting an appropriate therapy for a subject, the method involving genotyping CES1 in a subject, where a genotype having a variant allele having reduced carboxylesterase activity or expression relative to a reference indicates that drug therapy is not appropriate for the subject, and where a genotype that is homozygous wild-type indicates that drug therapy is appropriate for the subject.

In yet another aspect, the invention provides a method of identifying a subject as responsive to drug therapy, the method involving genotyping CES1 in a subject, where a genotype having a variant allele having reduced carboxylesterase activity or expression relative to a reference identifies the subject as not responsive to drug therapy, and where a genotype that is homozygous wild-type identifies the subject as responsive to drug therapy.

In another aspect, the invention provides a method of monitoring drug therapy in a subject, the method involving genotyping CES1 in a subject, identifying a subject as having a variant allele of CES1, and measuring plasma levels of the drug in the subject to determine an appropriate dosage.

In still another aspect, the invention provides a method of characterizing subject sensitivity to a drug, the method comprising genotyping CES1 in a subject, where a genotype having a variant allele having reduced carboxylesterase activity or expression relative to a reference identifies the subject as sensitive to the drug, and where a genotype that is homozygous wild-type identifies the subject as not sensitive to the drug.

In an additional aspect, the invention provides a method of genotyping a subject, the method involving amplifying a CES1 nucleic acid in a biological sample from the subject by long range polymerase chain reaction (PCR); and detecting a variant allele of CES1 relative to a wild-type reference sequence, where the variant allele encodes a CES1 polypeptide having reduced carboxylesterase activity or expression relative to a reference selected from the group consisting of Gly143Glu and Asp260Glu frameshift.

In a further aspect, the invention provides a kit for genotyping a CES1 isoform in a subject containing a set of nucleic acid probes, where the nucleic acid probes can selectively bind to and amplify a nucleic acid at a CES1 isoform locus.

In various embodiments of any of the aspects delineated herein, the CES1 or CES1 isoform is one or more of a CES1A1, CES1A2, or CES1A3 isoform. In particular embodiments, the CES1 or CES1 isoform is a CES1A1 isoform. In various embodiments of any of the aspects delineated herein, the method distinguishes a heterozygous genotype from a homozygous genotype. In various embodiments of any of the aspects delineated herein, the subject is heterozygous or homozygous for a variant allele having reduced carboxylesterase activity or expression relative to a reference. In various embodiments of any of the aspects delineated herein, analyzing comprises nucleic acid sequencing, allele-specific hybridization, allele-specific PCR, oligonucleotide microarray analysis, or mass spectrometry.

In various embodiments of any of the aspects delineated herein, the variant allele encodes a CES1 polypeptide having reduced or increased carboxylesterase activity or expression relative to a reference. In various embodiments of any of the aspects delineated herein, the method identifies at least one alteration in an evolutionarily conserved residue of CES1. In various embodiments, the conserved residue is in a triad residue, in the active site, or in the oxyanion hole. In particular embodiments, the conserved residue serine 221 (S), glutamic acid 354 (E), histidine 468, or Gly141-143.

In various embodiments, the alteration is in a residue that stabilizes substrate-enzyme intermediates or that alters enzyme activity. In particular embodiments, the variant allele is selected from the group consisting of Gly143Glu and Asp260Glu frameshift. In other embodiments, the variant allele is one or more of an allele set forth in Table 1.

In various embodiments of any of the aspects delineated herein, the variant allele is indicated by the presence of a single nucleotide polymorphism (SNP). In particular embodiments, the SNP is selected from the group consisting of 428G>A and T891del. In other embodiments, the SNP is one or more of a SNP set forth in Table 1.

In various embodiments of any of the aspects delineated herein, the method further comprises assaying the carboxylesterase activity of the protein encoded by the variant allele of CES1. In various embodiments of any of the aspects delineated herein, the method further comprises assaying substrate hydrolysis for the variant allele of CES1. In various embodiments of any of the aspects delineated herein, substrate hydrolysis is determined by contacting a drug with the protein and detecting the half-life (t_{1/2}), pharmacokinetics of the drug, or altered pharmacodynamic (PD) effect of the drug.

In various embodiments of any of the aspects delineated herein, the method involves selecting an appropriate drug therapy for the subject. A genotype comprising the presence of a variant allele having reduced carboxylesterase activity or expression relative to a reference identifies the subject as sensitive to the drug. A genotype that is homozygous wild-type identifies the subject as not sensitive to the drug. In various embodiments of any of the aspects delineated herein, the subject is administered an effective amount of the drug. In other embodiments of any of the aspects delineated herein, drug administration to the subject is discontinued.

In various embodiments of any of the aspects delineated herein, the drug is a compound selected from the group consisting of a prodrug, an illicit drug, an opioid, a dopaminergic or noradrenergic drug, an ACE Inhibitor, an HMG-CoA reductase inhibitor, a statin, an anesthetic, a toxin, a chemical warfare agent, an insecticide (e.g., an organophosphate insecticide), an antiviral drug, and an anti-cancer drug. In various embodiments of any of the aspects delineated herein, the drug is a compound selected from the group consisting of lidocaine, cilazapril, delapril, imidapril, cocaine, enalapril, quinapril, temocapril, methylphenidate, benazepril, trandolapril, lovastatin, oseltamivir, meperidine, prasugrel, simvastatin, valacyclovir, capecitabine, heroin, clopidogrel, sarin, soman, tabun, cholesterol, irinotecan (CPT-11), and mycophenolate.

In various embodiments of any of the aspects delineated herein, the drug is a prodrug that requires CES1 activity for bioactivation. In specific embodiments, the prodrug is a compound selected from the list consisting of oseltamivir, cilazapril, enalapril, capecitabine, delapril, quinapril, imidapril, temocapril, and lovastatin. In various embodiments of any of the aspects delineated herein, the method further comprises selecting an alternative therapy.

In various embodiments of any of the aspects delineated herein, the subject is identified as having an adverse reaction to a drug that requires CES1 activity for bioinactivation. In some embodiments, the drug is a compound selected from the list consisting of cocaine, methylphenidate, meperidine, capecitabine, and clopidogrel. In other embodiments, the drug is a compound selected from the list consisting of an insecticide, an organophosphate insecticide, or paraoxon.

In various embodiments of any of the aspects delineated herein, the subject is heterozygous or homozygous for a variant allele having reduced carboxylesterase activity or expression relative to a reference. In various embodiments of any of the aspects delineated herein, genotyping CES1 in a subject involves amplifying a CES1 nucleic acid by long range polymerase chain reaction (PCR) and analyzing the sequence of the amplified nucleic acid for a variant allele of CES1 relative to a wild-type reference sequence. In various embodiments of any of the aspects delineated herein, the method further involves decreasing a dosage of a drug administered to the subject or discontinuing treatment in the subject.

In various embodiments of any of the aspects delineated herein, the kit contains a nucleic acid probe set forth in Table 3. In various embodiments of any of the aspects delineated herein, the kit contains instructions for selecting an appropriate therapy for a subject, identifying a subject as responsive to drug therapy, monitoring drug therapy in a subject, or characterizing subject sensitivity to a drug based on the genotype of a CES1 isoform of the subject.

In various embodiments of any of the aspects delineated herein, the kit contains a microarray for detecting an SNP in a gene encoding an enzyme involved in drug metabolism or a drug transporter. In specific embodiments, the enzyme is one or more of cytochrome P450 2D6 and cytochrome P450 2C19.

Other features and advantages of the invention will be apparent from the detailed description, and from the claims.

### DEFINITIONS

Unless defined otherwise, all technical and scientific terms used herein have the meaning commonly understood by a person skilled in the art to which this invention belongs.

The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “alteration” is meant an increase or decrease. An alteration may be by as little as 1%, 2%, 3%, 4%, 5%, 10%, 20%, 30%, or by 40%, 50%, 60%, or even by as much as 75%, 80%, 90%, or 100%.

By “allele” is meant one of a series of two or more different gene sequences that occupy the same position or locus on a chromosome.

By “amplify” is meant to increase the number of copies of a molecule. In one example, the polymerase chain reaction (PCR) is used to amplify nucleic acids.

By “binding” is meant having a physicochemical affinity for a molecule. Binding is measured by any of the methods of the invention, e.g., hybridization of a detectable nucleic acid probe, such as a TaqMan® based probe, Pleiades based probe.

By “biological sample” is meant any tissue, cell, fluid, or other material derived from an organism (e.g., human subject).

By “bioactivation” is meant the chemical alteration of a compound within a subject to generate a compound or form of a compound having a pharmacological effect. For example, a prodrug undergoes bioactivation to remove protective groups, thereby producing a drug in active form (e.g., increasing the pharmacodynamic (PD) effect of the drug). In the context of the invention, the hCES1 enzyme bioactivates specific prodrugs and/or selected non-prodrugs by carboxylesterase catalytic activity (hydrolysis of a carboxylic acid ester, carbamate, thioester, or amide).

By “bioinactivation” is meant the chemical alteration of a compound within a subject to decrease or eliminate the pharmacological effect of the compound or otherwise serve in a detoxifying capacity. For example, bioinactivation results in a decrease in the half-life (t_{1/2}) of the drug or a decrease in the pharmacodynamic (PD) effect of the drug. In the context of the invention, the CES1 enzyme bioinactivates compounds by carboxylesterase activity (hydrolysis of a carboxylic acid ester, carbamate, thioester, or amide).

By “biomarker” is meant a polypeptide or polynucleotide that is differentially present in a sample taken from a subject having a disease or disorder relative to a reference. Exemplary biomarkers include nucleic acid molecules encoding variant alleles.

By “carboxylesterase 1 polypeptide” or “CES1 polypeptide” is meant a polypeptide or fragment thereof having at least 85% amino acid identity to NCBI Accession No. AA110339 and having carboxylesterase activity.

By “carboxylesterase 1 nucleic acid molecule” or “CES1 nucleic acid molecule” is meant a polynucleotide encoding a CES1 polypeptide. Exemplary CES1 nucleic acid molecules are provided at NCBI Accession Nos. AB119997 (CES1A1); AB 119998 (CES1A2); and NT_{—}010498 (CES1A3).

By “carboxylesterase activity” is meant the hydrolysis of a carboxylic acid ester, carbamate, thioester, or amide containing agent.

By “detect” refers to identifying the presence, absence, level, or concentration of an agent.

By “detectable” is meant a moiety that when linked to a molecule of interest renders the latter detectable. Such detection may be via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “drug” is meant a chemical compound, composition, agent (e.g., a pharmaceutical agent) capable of inducing a pharmacological effect in a subject. A drug when properly administered to a patient as a pharmaceutical agent has a desired therapeutic effect. Additionally, the term “drug” also encompasses drugs regarded as illicit or having the potential for abuse, such as cocaine, meperidine (Demerol®) or heroin.

By “genotype” is meant the genetic composition of a cell, organism, or individual. With reference to the invention, the genotype of an individual is determined as heterozygous or homozygous for one or more variant alleles of interest.

By “genotyping” is meant the characterization of the two alleles in one or more genes of interest (i.e., to determine a genotype).

By “heterozygous” is meant that a chromosomal locus has two different alleles. In one embodiment of the invention, heterozygous refers to a genotype in which one allele has a wild-type CES1 sequence (e.g., encoding a CES1 that has carboxylesterase activity) and the other allele has a sequence encoding a CES1 variant that does not have carboxylesterase activity (e.g., an alteration in serine 221 (S), glutamic acid 354 (E), histidine 468 (H), Gly141-143 (G), Asp260Glu frameshift).

By “homozygous” is meant that a chromosomal locus has two identical alleles. In the invention, homozygous wild-type is meant to refer to a genotype in which both alleles have a wild-type CES1 sequence (e.g., encoding a CES1 that has carboxylesterase activity). In some embodiments, homozygous can refer to a genotype in which both alleles have a sequence encoding a CES1 variant that does not have carboxylesterase activity (e.g., an alteration in serine 221 (S), glutamic acid 354 (E), histidine 468 (H), Gly141-143 (G), Asp260Glu frameshift). In particular embodiments, the inactive CES1 variant alleles are identical at one or more SNPs.

By “increases” is meant a positive alteration of at least 10%, 25%, 50%, 75%, 100%, 200%, 300%, 400%, 500%, 1000%, or more.

By “long range polymerase chain reaction” or “long range PCR” is meant the amplification of a nucleic acid having a length of 4, 5, 6, 7, 8, 9, 10, 11, 12, 13, 14, 15, 16, 167, 18, 19, 20, 21, 22, 23, 24, 25 kb, or more. Long range PCR involves one or more thermostable DNA polymerases, any one of which may have 3′→5′ exonuclease activity.

By “marker” is meant any protein or polynucleotide having an alteration in activity, expression level, or sequence that is associated with a disease, disorder, or condition.

By “native” is meant endogenous, or originating in a sample.

As used herein a “nucleic acid or oligonucleotide probe” is defined as a nucleic acid capable of binding to a target nucleic acid of complementary sequence through one or more types of chemical bonds, usually through complementary base pairing, usually through hydrogen bond formation. As used herein, a probe may include natural (i.e., A, G, C, or T) or modified bases (7-deazaguanosine, inosine, etc.). In addition, the bases in a probe may be joined by a linkage other than a phosphodiester bond, so long as it does not interfere with hybridization. It will be understood by one of skill in the art that probes may bind target sequences lacking complete complementarity with the probe sequence depending upon the stringency of the hybridization conditions. The probes are preferably directly labeled with isotopes, for example, chromophores, lumiphores, chromogens, or indirectly labeled with biotin to which a streptavidin complex may later bind. By assaying for the presence or absence of the probe, one can detect the presence or absence of a target gene of interest.

By “prodrug” is meant any compound that must undergo bioactivation before exhibiting its intended pharmacological effects. Prodrugs can be viewed as compounds which have incorporated specialized non-toxic protective groups which are intended to exist only transiently to alter or eliminate undesirable characteristics of the active compound. Such undesirable qualities or impediments to adequate delivery of the therapeutic moiety to the intended site of action often relate to poor aqueous solubility, absorption and permeability as well as high first-pass hepatic extraction-all factors which contribute to overall poor oral bioavailability.

By “reduces” is meant a negative alteration of at least 10%, 25%, 50%, 75%, or 100%.

By “reference” is meant a standard or control condition. In various embodiments of the invention, the reference is the wild-type sequence of a gene or gene isoform.

By “subject sensitivity” is meant the pharmacological response of a subject to a compound or drug. From a drug metabolism perspective, an increase in subject sensitivity may result from a decrease in bioactivation and/or a decrease in bioinactivation of a specific compound. For example, a decrease in bioactivation of a prodrug or a decrease in bioinactivation of a drug may lead to an accumulation of the inactive prodrug, as well as non-prodrug agents which serve as hCES1 substrates. Such accumulation may lead to unanticipated concentrations of these agents following typical clinical dosing which may lead to adverse side effects, as well as the potential for toxicity in a given patient. Furthermore, in the case of prodrugs, the lack of requisite bioactivation would likely lead to therapeutic failure since the active moiety may be only minimally liberated from the prodrug, or not liberated at all. In the context of the invention, CES1 enzyme activity is indicative of subject sensitivity. Subjects having one or more variant alleles at a CES1 locus (e.g., CES1A1) have reduced CES1 enzyme activity. In a particular embodiment, a decrease in bioinactivation of an insecticide due to reduced CES1 enzyme activity causes subject sensitivity when the insecticide accumulates to toxic levels in the subject.

By “responsive” or “responsiveness” is meant that a subject to which a compound is administered will obtain the pharmacological effect of the compound. A decrease in responsiveness may result from a decrease or complete loss in bioactivation or an increase in bioinactivation of a compound. For example, a decrease in bioactivation of an administered prodrug results in a decrease in systemic concentrations of the intended active compound such that the subject does not obtain or benefit from the intended pharmacological effect(s). In the context of the invention, CES1 enzyme activity is indicative of subject responsiveness. Subjects having two wild-type alleles at a CES1 locus (e.g., CES1A1) have normal CES1 enzyme activity and are responsive. Subjects having one or more variant alleles at a CES1 locus (e.g., CES1A1) have reduced CES1 enzyme activity and are not responsive. In one embodiment, a subject having normal CES1 enzyme activity indicates that drug therapy is appropriate. In another embodiment, a subject having reduced CES1 enzyme activity indicates that drug therapy is inappropriate.

The phrase “selectively (or specifically) hybridizes to” refers to the binding, duplexing, or hybridizing of a molecule only to a particular nucleotide sequence under stringent hybridization conditions when that sequence is present in a complex mixture (for example, total cellular or library DNA or RNA).

By “single nucleotide polymorphism” or “SNP” is meant a DNA sequence variation occurring when a single nucleotide in the genome differs between members of a biological species or paired chromosomes in an individual. SNPs are used as genetic markers for variant alleles.

By “target nucleic acid molecule” is meant a nucleic acid or biomarker of the sample that is to be detected.

By “variant” as is meant a polynucleotide or polypeptide sequence that differs from a wild-type or reference sequence by one or more nucleotides or one or more amino acids. Exemplary CES1 variants include CES1A1, CES1A2, and CES1A3.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

### BRIEF DESCRIPTION OF THE DRAWINGS

FIGS. 1A and 1B depict genotyping of individuals heterozygous for CES1A1 428G>A (Gly143Glu) allele, which is a marker for poor drug metabolism. FIG. 1A is a gel showing PCR amplification of ˜14 kb fragments from CES1A and CES1A3/CES1A2 genes. FIG. 1B depicts sequencing chromatograms of CES1A exon 4 of a poor metabolizer (PM) and the biological parents, indicating that the PM and the father are heterozygous for CES1A1 428G>A (Gly143Glu) and that the mother is wild-type (WT).

FIG. 2 is a DNA sequencing chromatogram depicting the identification of an individual homozygous for the CES1A1Gly143Glu (428G>A) allele.

FIG. 3 is a graph depicting the detection of variant Gly143Glu (428G>A) of CES1A 1 using a previously developed Taqman® assay. The Taqman® assay was not able to distinguish the homozygote from the heterozygotes.

FIG. 4 provides exemplary sequences of human CES1A1, CES1A2, and CES1A3 polypeptides and nucleic acid molecules.

### DETAILED DESCRIPTION OF THE INVENTION

As described below, the present invention features compositions and methods that provide for genotyping CES1 isoforms (CES1A1, CES1A2, and CES1A3).

Advantageously, the present invention provides genotype information for one or more of CES1A1, CES1A2, and CES1A3 genes. The invention is based, at least in part, on the discovery that genotyping CES1A1 alleles characterizes carboxylesterase activity in a subject. A reduced level of carboxylesterase activity in a subject is indicative of abnormal drug metabolism. Current Taqman®-based methods, which employ hydrolysis probes relying on the 5′-3′ exonuclease activity of Taq polymerase to cleave a dual-labeled probe during hybridization to the complementary target sequence (with fluorophore-based detection) are incapable of distinguishing variants between CES1 gene isoforms due to the high similarity of their DNA sequences. The current Taqman®-based methods for detecting CES1 alleles are incapable of distinguishing between homozygous and heterozygous genotypes for one or more of the CES1 gene isoforms (CES1A1, CES1A2, and CES1A3). The results presented herein describe the successful application of a novel specific CES1 genotyping assay to human DNA samples. Genotyping performed in accordance with the methods of the invention are useful for the diagnosis, monitoring, or characterization of virtually any disease, disorder, or condition characterized by an alteration in nucleic acid sequence in a gene having high sequence similarity to one or more genes, for example, CES1A1, CES1A2, and CES1A3.

### Mammalian Carboxylesterases (CESs)

Mammalian carboxylesterases (CESs) are members of the α/β-hydrolase family of proteins encoded by multiple genes (Imai et al., 2006) and ˜1% of the human proteome is comprised of serine hydrolases (Ross and Crow, 2007). CESs assume prominent roles in the hydrolysis of numerous and diverse compounds including carboxylic acid esters, carbamates, thioesters, and amide containing agents. These substrates are represented in every therapeutic drug class inclusive of many prodrugs, agents purposely formulated as esters for the purpose of improving oral bioavailability of the active moiety. Further, endogenous substrates such as triacylglycerols, fatty acyl-CoA esters, and some environmental toxins have been identified as substrates (Satoh et al., 2002). In general, CESs catalyze the conversion of lipophilic ester substrates into more water-soluble carboxylic acids facilitating their elimination (Brzezinski et al., 1997). CES-mediated hydrolysis is a catabolic process that transforms an active substrate to an inactive metabolite (e.g., metabolism of methylphenidate or clopidogrel (Plavix®), or yields more active products (e.g., conversion of heroin to monoacetylmorphine and morphine) (Brzezinski et al., 1997; Bencharit et al., 2003; Bencharit et al., 2006; Imai, 2006). The expression of CESs is generally highest in the epithelia of most organs within the endoplasmic reticulum. Localization suggests that CES enzymes provides a protective function against toxins. The highest CES hydrolytic activity occurs in the liver.

At least four main groups of CESs (CES1-CES4) as well as several subgroups are recognized in humans according to the homology of the amino acid sequence (Imai, 2006). However, two human isoenzymes hCES1 and hCES2, which belong to classes CES1 and CES2, respectively, are relevant to human drug metabolism. hCES1 and hCES2 are known to exhibit broad substrate specificities. In some cases, both hCES1 and hCES2 contribute sequentially to the metabolism of the same molecule (e.g. irinotecan [CPT-11]). However, significant differences exist between hCES1 and hCES2 relative to localization, substrate specificity, immunological properties and gene regulation (Satoh and Hosokawa, 1998; Imai et al., 2006).

Human Carboxylesterase 1 (hCES1)

hCES1 is the most abundant carboxylesterase expressed in the human liver (˜50-fold greater than hCES2), and contributes approximately 80% of total hepatic hydrolytic activity (Imai, 2006). hCES1 is essential for the activation of a number of specific prodrugs, such as oseltamivir, trandolapril (Shi et al., 2006; Zhu et al., 2009b). Additionally, hCES1, (but not hCES2) catalyzes transesterification reactions with ester drugs and endogenous substrates when ethanol is co-ingested (i.e. forming ethyl-esters). Transesterification products include a variety of agents which can be active, inactive, or potentially toxic (Bencharit et al., 2003; Bencharit et al., 2006). hCES1 is also known to exhibit stereoselectivity for a number of substrates, such as methylphenidate (MPH) and cocaine (Brzezinski et al., 1997; Sun et al., 2004). Drug metabolism and dispositional data generated in animal models is problematic due to interspecies differences in CESs (Hosokawa, 2010). For example, humans are one of the few mammals that have no plasma carboxylesterase activity, and even non-human primates differ significantly from humans relative to tissue expression of CES1 and CES2.

hCES1 is encoded in humans by the CES1 gene. Three isoforms of the CES1 gene have been identified, i.e. CES1A1, CES1A2, and CES1A3 (Hosokawa et al., 2007; Fukami et al., 2008). CES1A1 and CES1A3 are inversely located on chromosome 16 q13-q22.1 while CES1A2 is a variant of the CES1A3 gene (Fukami et al., 2008). Both CES1A1 and CES1A2 are functional whereas CES1A3 is a pseudogene due to a premature stop codon located in exon 3. CES1A1 is identical to CES1A2 except its promoter region and exon 1 (Fukami et al., 2008). CES1A) and CES1A2 isoforms produce an identical mature protein. In the liver, the majority of hCES1 is the product of the CES1A1 gene because the transcription efficiency of the CES1A2 gene is substantially lower than that of the CES1A1 due to the different promoter structures associated with the two genes (Fukami et al., 2008; Hosokawa et al., 2008).

CES1A1 variants have a more significant influence on enzyme activity than CES1A2 and CES1A3 variants, which contribute less to hCES1 activity. Thus, genetic variants present in CES1A1 have a greater impact on hCES1 function than when the same variants are present in CES1A2 or CES1A3.

The expression and activity of hCES1 exhibit substantial interindividual variability (Yang et al., 2009; Zhu et al., 2009a). Genetic variation is a contributing factor to varied hCES1 function in humans. Significant adverse effects can result as a consequence of variable metabolism or pharmacokinetics of a drug between individuals. Adverse effects (e.g., toxicity, etc.) may occur when a typically therapeutic amount or dose of a medicinal or therapeutic agent, e.g., methylphenidate (Ritalin®) is administered to an individual and results in elevated systemic concentrations (e.g., blood concentration), and attendant CNS concentrations, due to altered metabolism of the therapeutic agent. For example, therapeutic doses of di-methylphenidate can occasionally cause significant increases in a number of cardiovascular parameters due to both central dopaminergic effects and increased plasma epinephrine concentrations. In rare instances, stroke or sudden death have been reported in patients with underlying risk factors and has led to the United States Food and Drug Administration to mandate that drug manufacturers of psychostimulants provide educational literature on the risks to patients. In the instance of the prescribing and administering of a prodrug serving as a hCES1 substrate, such as the antiviral oseltamivir (Tamiflu®), a patient with deficient hCES1 activity is subject to risks of both therapeutic failure (in lieu of requisite bioactivation to active drug), and potential toxicity secondary to the unintended accumulation of the non-hydrolyzed prodrug.

Previous studies identified two novel SNPs Gly143Glu and Asp260fs within the CES1A1 and CES1A2 genes, respectively, in a normal volunteer who participated in a pharmacokinetic study of dl-MPH (Patrick et al., 2007; Zhu et al., 2008). The subject was found to have enormous systemic blood concentrations of both d- and I-MPH and an unprecedented enrichment of l-MPH relative to d-MPH following a single modest dose (0.3 mg/kg) (Patrick et al., 2007). Additionally, a prolonged half-life (t_{1/2}) and significantly altered pharmacodynamic (PD) effects were observed (Zhu et al., 2008). Subsequent investigations have revealed the minor allele frequency (MAF) of Gly143Glu to be 3.7%, 4.3%, 2.0, and 0% in Caucasian, Black, Hispanic, and Asian populations, respectively. The Asp260fs variant was extremely rare as none of the 925 screened subjects carried this mutation.

The functional consequences of both mutations have been investigated utilizing cell lines stably expressing each mutant (Zhu et al., 2008; Zhu et al., 2009b; Zhu and Markowitz, 2009). Briefly, cell lines stably expressing wild-type or a mutant CES1 are established using standard co-transfection methods, and a cleared supernatant from lysates of each of the cell lines is prepared. Hydrolysis reactions of a substrate (e.g., p-nitrophenol acetate (PNP), methylphenidate (MPH)) are performed using the CES1 wild-type and CES1 mutant supernatants, using a range of substrate concentrations. Collected data, accounting for spontaneous hydrolysis, were fit to the Michaelis-Menten equation, and kinetic parameters were calculated with nonlinear regression analysis with Graphpad Prism software (Graphpad Software, San Diego, Calif., USA).

In vitro incubation studies demonstrated that the catalytic function of both Gly143Glu and Asp260fs was profoundly impaired in terms of hydrolyzing the hCES1 selective substrates MPH, trandolapril, and oseltamivir (Zhu et al., 2008; Zhu et al., 2009b; Zhu and Markowitz, 2009). This finding represented the first direct correlation between a CES1 mutation and aberrant drug concentration of a medication in a human subject.

A clinical study investigating the association of the Gly143Glu mutation with response to MPH therapy in ADHD patients (Nemoda et al., 2009) expands and supports Applicants' discoveries regarding the function of Gly143Glu in CES1 activity. Preliminary data from the study demonstrated that patients carrying the Gly143Glu variant required significantly lower doses of MPH for symptom reduction relative to the subjects carrying WT CES1 (0.410±0.127 vs. 0.572±0.153 mg/kg, t(1,88)=2.33, p=0.022). However, this study did not examine MPH blood concentrations (Nemoda et al., 2009).

In addition to the novel variants, the CES1 SNPs Cyc88Phe, and Arg200His have been determined to exert significantly less catalytic activity, while Ser76Asn exhibits slightly higher enzymatic activity relative to WT enzyme in vitro (Shi et al., 2006). To date, a total of 26 nonsynonymous SNPs of CES1 have been recorded in the NCBI dbSNP database (Table 1). Except for the two variants (rs71647871 and rs71647872) identified by the Applicants, the genomic locations (in CES1A1, CES1A2, or CES1A3 genes) of the CES1 variants listed in Table 1 have not been determined.

TABLE 1
Nonsynonymous CES1 variants

| Region | dbSNP rs# | Mutations | Minor Allele Frequency |
|---|---|---|---|
| Exon 1 | rs111604615 | G119C, Arg4Pro | NA |
| | rs114788146 | A127G, Ile7Val | NA |
| | rs116258771 | C139A, Leu11Ile | NA |
| | rs28563878 | G142T, Ser12Ala | NA |
| Exon 2 | rs3826190 | G164T, Gly19Val | NA |
| | rs3826191 | C208A, Leu34Met; | NA |
| | | C208G, Leu34Val | |
| | rs3826192 | G223A, Val39Ile | NA |
| | rs72808055 | A256G, Ile50Val | NA |
| | rs3177828 | C278A, Ala57Gly | NA |
| | rs75463934 | C284T, Pro59Leu | NA |
| | rs2307240 | G335A, Ser76Asn | NIHPDR: 0.051; CEU: |
| | | | 0.034, HCB: 0.068, |
| | | | JPT: 0.023; YRI: 0.009 |
| | rs62028647 | C356T, Ser83Leu | NA |
| Exon 3 | rs5023782 | G371T, Cyc88Phe | NA |
| | rs5023781 | C400A, Leu98Ile | NA |
| | rs5023780 | C421T, Arg105X | NA |
| | rs28760313 | C449T, Ser114Phe | NA |
| Intron 3 | rs469103814 | T9298C | W: 0.028, B: 0.000, |
| | | | O: 0.000 |
| | rs469103815 | A9324G | W: 0.023, B: 0.000, |
| | | | O: 0.000 |
| | rs469103816 | T9386C | W: 0.000, B: 0.071, |
| | | | O: 0.000 |
| | rs469103817 | C9387A | W: 0.011, B: 0.000, |
| | | | O: 0.000 |
| Exon 4 | rs71647871 | G539A, Gly144Glu | W: 0.037, B: 0.043, |
| | | | H: 0.020, A: 0.000 |
| | rs4784575 | G629A, Gly174Asp | NA |
| Exon 5 | rs60054861 | G668C, Arg187Pro | NA |
| Intron 3 | rs469103818 | C9604T | W: 0.011, B: 0.321, |
| | | | O: 0.000 |
| | rs469103819 | G9635A | W: 0.006, B: 0.000, |
| | | | O: 0.000 |
| | rs2307241 | G668del, Arg187fs | NIHPDR: 0.013 |
| | rs2307243 | G707A, Arg200His | NIHPDR: 0.012 |
| | rs2307227 | C720A, Asp204Glu | NIHPDR: 0.037; CEU: |
| | | | 0.018; HCB: 0.045; |
| | | | JPN: 0; YRI: 0.043 |
| Exon 6 | rs71647872 | T891del, Asp261fs | NA |
| Exon 7 | rs115629050 | G913T, Ala289Ser | NA |
| | rs114119971 | C963G, His285Gln | NA |
| Exon 11 | rs114277361 | T1406C, Ile433Thr | NA |

CEU: HapMap Utah residents with Northern and Western European ancestry from the CEPH collection
HCB: HapMap Han Chinese in Beijing
JPN: HapMap Japanese in Tokyo
YUI: HapMap Yoruba in Ibadan
NIHPDR: the NIH polymorphism discovery resource
W: White; B: Black; H: Hispanic; A: Asian; O: Other than White, Black

A recently published study reported a number of novel SNPs in both CES1A1 and CES1A2 genes (Yamada et al., 2010). However, based upon the primers used in this report, the investigators were not able to distinguish between CES1A1 and CES1A3/CES1A2 genes. For instance, alignment analysis of CES1A1 (AB 11995) and CES1A2 (AB 11996) cDNA suggested that nonsynonymous SNPs (11G>C, 15C>T, 17T>C, and 19A>G) within CES1A1 exon 1 were not CES1A1 variants but rather the wild-type CES1A2 gene. Thus, genotyping of the nonsynonymous and promoter SNPs reported in the study need to be performed to determine the location of the CES1 SNPs in CES1A1, CES1A2. CES1A3 isoforms.

In addition to nonsynonymous SNPs, a total of 11 SNPs within the promoter region of the CES1A2 gene were identified and reported in a recent study (Yoshimura et al., 2008). Among them, −816A>C was determined to be significantly associated with the efficacy of the ACE-inhibitor imidapril, a prodrug activated by hCES1 in humans (Geshi et al., 2005). In addition to these promoter and nonsynonymous mutations, a significant number of other CES1 variants, such as synonymous mutants and SNPs within introns, have been documented in several SNPs databases. However, no functional consequences or clinical significance have been attributed to these mutations to date.

CES1 Genetic Variants and hCES1 Structure and Function

The hCES1 enzyme belongs to a larger family of serine hydrolases, which include human acetylcholinesterase (AcChE) and butyrylcholine esterase (BuChE). Crystal structures of human CES1, AcChE, and BuChE indicate that each has an analogous active site groove containing a catalytic triad consisting of a serine, a glutamic acid, and a histidine residue (Fleming et al., 2005).

Glycine at position 143 of hCES1 is important for hCES1 protein function. For hCES1, the corresponding active site triad residues are serine 221 (S), glutamic acid 354 (E), and histidine 468. A series of three consecutive glycine residues are also located in the active site of hCES1 (Gly141-143) and create what is referred to as an “oxyanion hole.” The oxyanion hole is thought to stabilize substrate-enzyme intermediates via hydrogen bonds formed with the oxyanion form of the carbonyl oxygen and, thus, would be fundamental to proper hCES1 function (Fleming et al., 2005). The catalytic triad and oxyanion hole are evolutionarily conserved both across species (fish to humans) and within related serine hydrolases (Fleming et al., 2005). When the glycine in hBuChE analogous to Gly143 in hCES1 was mutated both substrate affinity and catalysis were markedly reduced or abolished (Masson et al., 2007). Without being bound to a particular theory, the mutation of Gly143 to glutamic acid (Gly143Glu) in hCES1 results in reduced carboxylesterase activity due to disruption of the oxyanion hole. hCES1 activity can be assessed by in vitro incubation assays previously described (Zhu et al., 2008; Zhu et al., 2009b; Zhu and Markowitz, 2009).

Similarly, the 12785T>del mutation (Asp260fs) causes a significant change in hCES1 structure. The deletion of nucleotide 780 in CES1 causes an early truncation and alteration of residues 260-299. Thus, this frameshift mutation eliminates two of the three conserved catalytic triad residues as well as other residues involved in protein function. Both of the polymorphisms Gly143Glu and Asp260fs, either alone or in combination, result in a significant decrease of loss of hCES1 activity. A slow metabolizer was identified as heterozygous for both mutations and each mutation occurred on a different allele. The identification of the slow metabolizer was instrumental in the discovery the two SNPs.

### Diagnostic Methods

The present invention provides a number of diagnostic assays that are useful for characterizing the genotype of a subject. The present invention can be employed to genotype a gene of interest in a subject, where the gene of interest has a similar isoform(s). Desirably, the methods of the invention discriminate between the genotype of a gene of interest and the genotype of the similar isoform(s). Preferably, both or all alleles corresponding to a gene of interest are identified. Accordingly, the invention provides for genotyping useful in virtually any clinical setting where conventional methods of analysis are used.

In various aspects, the methods of the invention determine or detect the CES1 genetic variants comprising the genotype of CES1A1 and distinguish the CES1A 1 genotype from those of CES1A2 and CES1A3. In contrast to previous methods for detecting CES1 allelic variants, the genotyping methods described herein are able to more accurately assess CES1 activity by examining the contributions of the CES1 isoforms, in particular CES1A1. The present methods provide a genetic means for the analysis of biomarkers in CES1 associated with drug metabolism. Results obtained from CES1 genotyping assays may be used to select an appropriate therapy for a subject, monitor drug therapy in a subject, identify a subject as responsive to drug therapy, or identify a subject as sensitive to a drug. This level of genotyping will better enable individualized pharmacogenetic-based therapy. In particular embodiments, the invention provides for the detection of CES1 allelic variants and SNPs listed in Table 1 (above). Advantageously, the methods of the invention distinguish between homozygous and heterozygous alleles of CES1.

### Types of Biological Samples

The genotyping methods of the invention involve detecting or determining a genetic variant or biomarker of interest in a biological sample. In one embodiment, the biologic sample contains a cell having diploid DNA content. Human cells containing 46 chromosomes (e.g., human somatic cells) are diploid. In one embodiment, the biologic sample is a tissue sample that includes diploid cells of a tissue (epithelial cells) or organ (e.g., skin cells). Such tissue is obtained, for example, from a cheek swab or biopsy of a tissue or organ. In another embodiment, the biologic sample is a biologic fluid sample. Biological fluid samples containing diploid cells include saliva, blood, blood serum, plasma, urine, hair follicle, or any other biological fluid useful in the methods of the invention.

### Genotyping of CES1 Polymorphisms

A CES1 isoform is amplified by long range PCR to determine the genotype of the isoform, e.g., CES1A1. The amplified nucleic acid corresponding CES1 isoform may be analyzed using a variety of methods for detecting variant alleles to determine the genotype. The presence or absence of one or both of the 12754T>del or Gly143Glu (428G>A) polymorphisms in the CES1 gene may be evaluated using various techniques. For example, the carboxylesterase-1 gene is amplified by long range PCR and sequenced to determine the presence or absence of a single nucleotide polymorphism (SNP). In certain embodiments, real-time PCR may be used to detect a single nucleotide polymorphism of the amplified products. In other embodiments, a polymorphism in the amplified products may be detected using a technique including hybridization with a probe specific for a single nucleotide polymorphism, restriction endonuclease digestion, primer extension, microarray or gene chip analysis, mass spectrometry, or a DNAse protection assay.

### Long Range Polymerase Chain Reaction (PCR)

Polymerase chain reaction (PCR) is widely known in the art. For example, U.S. Pat. Nos. 4,683,195, 4,683,202, and 4,800,159; K. Mullis, Cold Spring Harbor Symp. Quant. Biol., 51:263-273 (1986); and C. R. Newton & A. Graham, Introduction to Biotechniques: PCR, 2.sup.nd Ed., Springer-Verlag (New York: 1997), the disclosures of which are incorporated herein by reference, describe processes to amplify a nucleic acid sample target using PCR amplification extension primers which hybridize with the sample target. As the PCR amplification primers are extended, using a DNA polymerase (preferably thermostable), more sample target is made so that more primers can be used to repeat the process, thus amplifying the sample target sequence. Typically, the reaction conditions are cycled between those conducive to hybridization and nucleic acid polymerization, and those that result in the denaturation of duplex molecules.

In the first step of the reaction, the nucleic acid molecules of the sample are transiently heated, and then cooled, in order to denature double stranded molecules. Forward and reverse primers are present in the amplification reaction mixture at an excess concentration relative to the sample target. When the sample is incubated under conditions conducive to hybridization and polymerization, the primers hybridize to the complementary strand of the nucleic acid molecule at a position 3′ to the sequence of the region desired to be amplified that is the complement of the sequence whose amplification is desired. Upon hybridization, the 3′ ends of the primers are extended by the polymerase. The extension of the primer results in the synthesis of a DNA molecule having the exact sequence of the complement of the desired nucleic acid sample target. The PCR reaction is capable of exponentially amplifying the desired nucleic acid sequences, with a near doubling of the number of molecules having the desired sequence in each cycle. Thus, by permitting cycles of hybridization, polymerization, and denaturation, an exponential increase in the concentration of the desired nucleic acid molecule can be achieved.

The methods of the present invention involve amplifying large regions of a polynucleotide with high fidelity using a thermostable DNA polymerase having 3′→5′ exonuclease activity. As defined herein, “3′→5′ exonuclease activity” refers to the activity of a template-specific nucleic acid polymerase having a 3′→5′ exonuclease activity associated with some DNA polymerases, in which one or more nucleotides are removed from the 3′ end of an oligonucleotide in a sequential manner. Polymerase enzymes having high fidelity 3′→5′ exonuclease activity are useful, for example, when primer extension must be performed over long distances (i.e., when the desired PCR amplification product is greater than about 5 kb). Polymerase enzymes having 3′→5′ exonuclease proofreading activity are known to those in the art. Examples of suitable proofreading enzymes include TaKaRa LA Taq (Takara Shuzo Co., Ltd.) and Pfu (Stratagene), Vent, Deep Vent (New England Biolabs). Exemplary methods for performing long range PCR are disclosed, for example, in U.S. Pat. No. 5,436,149; Barnes, Proc. Natl. Acad. Sci. USA 91:2216-2220 (1994); Tellier et al., Methods in Molecular Biology, Vol. 226, PCR Protocols, 2nd Edition, pp. 173-177; and, Cheng et al., Proc. Natl. Acad. Sci. 91:5695-5699 (1994); the contents of which are incorporated herein by reference. In various embodiments, long range PCR involves one DNA polymerase. In some embodiments, long range PCR may involve more than one DNA polymerase. When using a combination of polymerases in long range PCR, it is preferable to include one polymerase having 3′→5° exonuclease activity, which assures high fidelity generation of the PCR product from the DNA template. Typically, a non-proofreading polymerase, which is the main polymerase is also used in conjunction with the proofreading polymerase in long range PCR reactions. Long range PCR can also be performed using commercially available kits, such as LA PCR kit available from Takara Bio Inc.

### Sequencing

DNA sequencing may be used to evaluate a polymorphism of the present invention. One DNA sequencing method is the Sanger method, which is also referred to as dideoxy sequencing or chain termination. The Sanger method is based on the use of dideoxynucleotides (ddNTP's) in addition to the normal nucleotides (NTP's) found in DNA. Dideoxynucleotides are essentially the same as nucleotides except they contain a hydrogen group on the 3′ carbon instead of a hydroxyl group (OH). These modified nucleotides, when integrated into a sequence, prevent the addition of further nucleotides. This occurs because a phosphodiester bond cannot form between the dideoxynucleotide and the next incoming nucleotide, and thus the DNA chain is terminated. Using this method, optionally coupled with amplification of the nucleic acid target, one can now rapidly sequence large numbers of target molecules, usually employing automated sequencing apparati. Such techniques are well known to those of skill in the art.

Pyrosequencing is another method of DNA sequencing that may be used to evaluate a polymorphism of the present invention, for example as described in U.S. Pat. Publ. No. 2006008824; herein incorporated by reference). Pyrosequencing, which is also referred to as sequencing by synthesis, involves taking a single strand of the DNA to be sequenced, synthesizing its complementary strand enzymatically one base pair at a time, and detecting by chemiluminescence the base that is added. In one embodiment, the template DNA is immobile, and solutions of A, C, G, and T nucleotides are sequentially added and removed from the reaction. Light is produced only when the nucleotide solution complements the first unpaired base of the template. The sequence of solutions which produce chemiluminescent signals allows the determination of the sequence of the template. The templates for pyrosequencing can be made both by solid phase template preparation (streptavidin-coated magnetic beads) and enzymatic template preparation (apyrase+exonuclease).

In a specific embodiment, ssDNA template is hybridized to a sequencing primer and incubated with the enzymes DNA polymerase, ATP sulfurylase, luciferase and apyrase, and with the substrates adenosine 5′ phosphosulfate (APS) and luciferin. The addition of one of the four deoxynucleotide triphosphates (dNTPs)(in place of dATP, dATPαS is added, which is not a substrate for a luciferase) initiates the second step. DNA polymerase incorporates the correct, complementary dNTPs onto the template, and the incorporation of the nucleotide releases pyrophosphate (PPi) stoichiometrically. ATP sulfurylase quantitatively converts PPi to ATP in the presence of adenosine 5′ phosphosulfate. The ATP generated acts to catalyze the luciferase-mediated conversion of luciferin to oxyluciferin and generates visible light in amounts that are proportional to the amount of ATP. The light produced in the luciferase-catalyzed reaction is detected by a camera and analyzed in a program. Unincorporated nucleotides and ATP are degraded by the apyrase, and the reaction can restart with another nucleotide.

Pyrosequencing, optionally coupled with amplification of the nucleic acid target, can sequence large numbers of target molecules, usually employing automated sequencing apparati, including long sequences (e.g., 400 million bp/10 hr in a single run).

Real-Time PCR (rtPCR)

The presence or absence of polymorphisms in CES1 isoforms may be detected using real-time PCR. Real-time PCR typically utilizes fluorescent probes for the selective detection of the polymorphisms. Various real-time PCR testing platforms that may be used with the present invention include: 5′ nuclease (TaqMan® probes), molecular beacons, and FRET hybridization probes. These detection methods rely on the transfer of light energy between two adjacent dye molecules, a process referred to as fluorescence resonance energy transfer (see, e.g., Espy et al (2006) Clin Microbiol Rev. 2006 January; 19(1): 165-256 for a review of various rtPCR approaches that may be used with the present invention).

### 5′ Nuclease Probes

In certain embodiments, a 5′ nuclease probe may be used to detect a polymorphism of the present invention. 5′ nuclease probes are often referred to by the proprietary name, TaqMan® probes. A TaqMan® probe is a short oligonucleotide (DNA) that contains a 5′ fluorescent dye and 3′ quenching dye. To generate a light signal (i.e., remove the effects of the quenching dye on the fluorescent dye), two events must occur. First, the probe must bind to a complementary strand of DNA, e.g., at about 60° C. Second, at this temperature, Taq polymerase, which is commonly used for PCR, must cleave the 5′ end of the TaqMan® probe (5′ nuclease activity), separating the fluorescent dye from the quenching dye.

In order to differentiate a single nucleotide polymorphism from a wild-type sequence in the DNA from a subject, a second probe with complementary nucleotide(s) to the polymorphism and a fluorescent dye with a different emission spectrum are typically utilized. Thus, these probes can be used to detect a specific, predefined polymorphism under the probe in the PCR amplification product. Two reaction vessels are typically used, one with a complementary probe to detect wild-type target DNA and another for detection of a specific nucleic acid sequence of a mutant strain. Because TaqMan® probes typically require temperatures of about 60° C. for efficient 5′ nuclease activity, the PCR may be cycled between about 90-95° C. and about 60° C. for amplification. In addition, the cleaved (free) fluorescent dye can accumulate after each PCR temperature cycle; thus, the dye can be measured at any time during the PCR cycling, including the hybridization step. In contrast, molecular beacons and FRET hybridization probes typically involve the measurement of fluorescence during the hybridization step.

Genotyping for the 12754T>del (“Asp260fs”) or Gly143Glu (428G>A, “Gly143Glu”) in the carboxylesterase-1 gene may be evaluated using the following (5′ endonuclease probe) real-time PCR technique. Genotyping assays can be performed in duplicate and analyzed on a Bio-Rad iCycler Iq® Multicolor Real-time detection system (Bio-Rad Laboratories, Hercules, Calif.). Real-time polymerase chain reaction (PCR) allelic discrimination assays to detect the presence or absence of specific single nucleotide polymorphisms in the CES1 gene, Gly143Glu (genomic: nt 9486; Cdna: nt 428) and Asp260fs (genomic: nt 12754; Cdna: nt 780), may utilize fluorogenic TaqMan® Probes.

Real-time PCR amplifications may be carried out in a 10 μl reaction mix containing 5 ng genomic DNA, 900 Nm of each primer, 200 Nm of each probe and 5 μl of 2xTaqMan® Universal PCR Master Mix (contains PCR buffer, passive reference dye ROX, deoxynucleotides, uridine, uracil-N-glycosylase and AmpliTaq Gold DNA polymerase; Perkin-Elmer, Applied Biosystems, Foster City, Calif.). Cycle parameters may be: 95° C. for 10 min, followed by 50 cycles of 92° C. for 15 sec and 60 C.° for 1 min. Real-time fluorescence detection can be performed during the 60° C. annealing/extension step of each cycle. The IQ software may be used to plot and automatically call genotypes based on a two parameter plot using fluorescence intensities of FAM and VIC at 49 cycles.

### Molecular Beacons

Molecular beacons are another real-time PCR approach which may be used to identify the presence or absence of a polymorphism of the present invention. Molecular beacons are oligonucleotide probes that are labeled with a fluorescent dye (typically on the 5′ end) and a quencher dye (typically on the 3′ end). A region at each end of the molecular beacon probe is designed to be complementary to itself, so at low temperatures the ends anneal, creating a hairpin structure. This hairpin structure positions the two dyes in close proximity, quenching the fluorescence from the reporter dye. The central region of the probe is designed to be complementary to a region of a PCR amplification product. At higher temperatures, both the PCR amplification product and probe are single stranded. As the temperature of the PCR is lowered, the central region of the molecular beacon probe may bind to the PCR product and force the separation of the fluorescent reporter dye from the quenching dye. Without the quencher dye in close proximity, a light signal from the reporter dye can be detected. If no PCR amplification product is available for binding, the probe can re-anneal to itself, bringing the reporter dye and quencher dye into close proximity, thus preventing fluorescent signal.

Two or more molecular beacon probes with different reporter dyes may be used for detecting single nucleotide polymorphisms. For example, a first molecular beacon designed with a first reporter dye may be used to indicate the presence of a SNP and a second molecular beacon designed with a second reporter dye may be used to indicate the presence of the corresponding wild-type sequence; in this way, different signals from the first and/or second reporter dyes may be used to determine if a subject is heterozygous for a SNP, homozygous for a SNP, or homozygous wild-type at the corresponding DNA region. By selection of appropriate PCR temperatures and/or extension of the probe length, a molecular beacons may bind to a target PCR product when a nucleotide polymorphism is present but at a slight cost of reduced specificity. Molecular beacons advantageously do not require thermocycling, so temperature optimization of the PCR is simplified.

### FRET Hybridization Probes

FRET hybridization probes, also referred to as LightCycler® probes, may also be used to detect a polymorphism of the present invention. FRET hybridization probes typically comprise two DNA probes designed to anneal next to each other in a head-to-tail configuration on the PCR product. Typically, the upstream probe has a fluorescent dye on the 3′ end and the downstream probe has an acceptor dye on the 5′ end. If both probes anneal to the target PCR product, fluorescence from the 3′ dye can be absorbed by the adjacent acceptor dye on the 5′ end of the second probe. As a result, the second dye is excited and can emit light at a third wavelength, which may be detected. If the two dyes do not come into close proximity in the absence of sufficient complimentary DNA, then FRET does not occur between the two dyes. The 3′ end of the second (downstream) probe may be phosphorylated to prevent it from being used as a primer by Taq during PCR amplification. The two probes may encompass a region of 40 to 50 DNA base pairs.

FRET hybridization probe technology permits melting curve analysis of the amplification product. If the temperature is slowly raised, probes annealing to the target PCR product will be reduced and the FRET signal will be lost. The temperature at which half the FRET signal is lost is referred to as the melting temperature of the probe system. A single nucleotide polymorphism in the target DNA under a hybridization FRET probe will still generate a signal, but the melting curve will display a lower Tm. The lowered Tm can indicate the presence of a specific polymorphism. The target PCR product is detected and the altered Tm informs the user there is a difference in the sequence being detected. Like molecular beacons, there is not a specific thermocycling temperature requirement for FRET hybridization probes. Like molecular beacons, FRET hybridization probes have the advantage of being recycled or conserved during PCR temperature cycling, and a fluorescent signal does not accumulate as PCR product accumulates after each PCR cycle.

### Primer Extension

Primer extension is another technique which may be used according to the present invention. A primer and no more than three NTPs may be combined with a polymerase and the target sequence, which serves as a template for amplification. By using less than all four NTPs, it is possible to omit one or more of the polymorphic nucleotides needed for incorporation at the polymorphic site. It is important for the practice of the present invention that the amplification be designed such that the omitted nucleotide(s) is(are) not required between the 3′ end of the primer and the target polymorphism. The primer is then extended by a nucleic acid polymerase, in a preferred embodiment by Taq polymerase. If the omitted NTP is required at the polymorphic site, the primer is extended up to the polymorphic site, at which point the polymerization ceases. However, if the omitted NTP is not required at the polymorphic site, the primer will be extended beyond the polymorphic site, creating a longer product. Detection of the extension products is based on, for example, separation by size/length which will thereby reveal which polymorphism is present. For example, U.S. Ser. No. 10/407,846, which is which is hereby incorporated by reference, describes a form of primer extension.

### RFLP

Restriction Fragment Length Polymorphism (RFLP) is a technique in which different DNA sequences may be differentiated by analysis of patterns derived from cleavage of that DNA. If two sequences differ in the distance between sites of cleavage of a particular restriction endonuclease, the length of the fragments produced will differ when the DNA is digested with a restriction enzyme. The similarity of the patterns generated can be used to differentiate species (and even strains) from one another.

Restriction endonucleases in turn are the enzymes that cleave DNA molecules at specific nucleotide sequences depending on the particular enzyme used. Enzyme recognition sites are usually 4 to 6 base pairs in length. Generally, the shorter the recognition sequence, the greater the number of fragments generated. If molecules differ in nucleotide sequence, fragments of different sizes may be generated. The fragments can be separated by gel electrophoresis. Restriction enzymes are isolated from a wide variety of bacterial genera and are thought to be part of the cell's defenses against invading bacterial viruses. Use of RFLP and restriction endonucleases in SNP analysis requires that the SNP affect cleavage of at least one restriction enzyme site.

### Mass Spectrometry

Mass spectrometry may also be used to detect a polymorphism of the present invention. By exploiting the intrinsic properties of mass and charge, mass spectrometry (MS) can resolved and confidently identified a wide variety of complex compounds. Traditional quantitative MS has used electrospray ionization (ESI) followed by tandem MS (MS/MS) (Chen et al., 2001; Zhong et al., 2001; Wu et al., 2000) while newer quantitative methods are being developed using matrix assisted laser desorption/ionization (MALDI) followed by time of flight (TOF) MS (Bucknall et al., 2002; Mirgorodskaya et al., 2000; Gobom et al., 2000). Methods of mass spectroscopy that may be used with the present invention include: ESI, ESI tandem mass spectroscopy (ESI/MS/MS), Secondary ion mass spectroscopy (SIMS), Laser desorption mass spectroscopy (LD-MS), Laser Desorption Laser Photoionization Mass Spectroscopy (LDLPMS), and MALDI-TOF-MS.

### Hybridization

There are a variety of ways by which one can assess genetic profiles, and may of these rely on nucleic acid hybridization. Hybridization is defined as the ability of a nucleic acid to selectively form duplex molecules with complementary stretches of DNAs and/or RNAs. Depending on the application envisioned, one would employ varying conditions of hybridization to achieve varying degrees of selectivity of the probe or primers for the target sequence.

Typically, a probe or primer of between 13 and 100 nucleotides, preferably between 17 and 100 nucleotides in length up to 1-2 kilobases or more in length will allow the formation of a duplex molecule that is both stable and selective. Such fragments may be readily prepared, for example, by directly synthesizing the fragment by chemical means or by introducing selected sequences into recombinant vectors for recombinant production.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

For certain applications, for example, lower stringency conditions may be used. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In certain embodiments, it will be advantageous to employ nucleic acids of defined sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known that can be employed to provide a detection means that is visibly or spectrophotometrically detectable, to identify specific hybridization with complementary nucleic acid containing samples.

In general, it is envisioned that the probes or primers described herein will be useful as reagents in solution hybridization, as in PCR, for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772. The relevant portions of these and other references identified in this section of the Specification are incorporated herein by reference.

### Pharmacogenetics: Consequence for Drug Therapy

Carboxylesterase-1 is important for the function and metabolism of many known compounds in humans and non-human animals. Thus, the presence or absence of one or more polymorphisms of the present invention may be used to “individualize” or modify a therapy for a subject or patient based on the sensitivity of the subject to a therapeutic due to the presence or absence of a polymorphism of the present invention.

The hCES1 enzyme catalyzes the hydrolysis of drugs from numerous classes. The hydrolysis generally produces inactive metabolite(s) (e.g., MPH and cocaine). However, hCES1 is also involved in the generation of active metabolites (e.g., conversion of heroin to monoacetylmorphine and morphine) or the activation of prodrugs such as the angiotensin converting enzyme (ACE) inhibitors quinapril and benazepril. Although minor overlap exists between CES1 and a related isoform CES2, only the CES1 isoform mediates transesterification reactions. hCES1 is the most abundant carboxylesterase expressed in the liver (˜50-fold higher than hCES2), and contributes approximately 80% of total hepatic hydrolytic activity (Imai, 2006). Of the CES1 isoforms identified CES1A1 variants influence CES1 hepatic hydrolytic activity more than CES1A2 variants, because transcription of the CES1A2 gene is substantially lower than that of the CES1A1 (Fukami et al., 2008; Hosokawa et al., 2008).

In certain embodiments, evaluating the presence or absence of a polymorphism of the present invention may be used to individualize a therapy and/or determine the sensitivity of a subject to a compound. The compound may be a prodrug, an illicit drug, an opioid, a dopaminergic or noradrenergic drug, an ACE Inhibitor, an HMG-CoA reductase inhibitor or “statin”, an anesthetic agent, an antiviral drug, or anti-cancer drug, a toxin, a chemical warfare agent, or certain insecticides, (e.g., an organophosphate). Examples of known hCES1 substrates, including compounds of the types listed above, are shown in Table 2.

TABLE 2
Examples of known hCES1 substrates

| | | | |
|---|---|---|---|
| lidocaine | cilazapril¹ | delapril¹ | imidapril¹ |
| cocaine² | enalapril¹ | quinapril¹ | temocapril¹ |
| methylphenidate² | benazepril | trandolapril | lovastatin¹ |
| oseltamivir¹ | meperidine² | prasugrel | simvastatin |
| valacyclovir | capecitabine^{1,2} | heroin | clopidogrel² |
| sarin⁵, soman⁵, tabun⁵ | cholesterol³ | irinotecan | mycophenolate |
| | | (CPT-11)^{1,4} | |

¹= prodrug substrate requiring metabolic activation,
²= agents subject to transesterification,
³= endogenous compound,
⁴= human CES-2 also contributes to biotransformation,
⁵= chemical warfare agents

With regard to drug abuse, the existence of an unrecognized hCES1 deficiency could potentially lead to idiosyncratic toxicities and/or fatal exposures (e.g., misinterpreted as intentional or accidental drug overdoses on the basis of antemortem or postmortem blood concentrations). For example, the potential for the role of dysfunctional hCES1 variants in such inaccurate conclusions is highlighted by the observation of a slow metabolizer participating as a normal volunteer in a pharmacokinetic study of methylphenidate metabolism and disposition. Following the administration of a single immediate-release weight-based dose of dl-methylphenidate, profound elevations in blood concentrations of methylphenidate were measured over time that were both unprecedented and far higher than any of 19 other study peers participating in the study. Furthermore, hydrolytic reactions can proceed on a stereoselective basis resulting in a distortion of the anticipated isomeric disposition of a racemic compound following the administration of a medication such as dl-methylphenidatem and such an effect was observed in this case. The potential risk for individuals with deficient hCES1 activity who abuse methylphenidate would be expected to be amplified given the likelihood of the self-administration of doses larger than typically prescribed and at more frequent intervals. Additionally, the crushing of tablets and nasal insufflation (i.e. snorting) of the powder are well documented. Such routes of administration would be expected to deliver even greater amounts of the drug to the systemic circulation and ensuing difficulties with biodeactivation.

Methylphenidate is the most common pharmacologic agent used to treat attention-deficit hyperactivity disorder which afflicts school-age children with an estimated worldwide prevalence of 8-12%. Significant interindividual variability in MPH pharmacokinetics and pharmacodynamics is well recognized yet remains unexplained. The most common formulation of methylphenidate is the racemic mixture of d-threo-(R,R)- and l-threo-(S,S)-methylphenidate (MPH) enantiomer, with the d-isomer regarded as the active therapeutic isomer. The primary metabolic pathway governing the metabolism of MPH is rapid deesterification to the inactive metabolite, ritalinic acid. This process is mediated by hCES1. Additionally, Sun and coworkers demonstrated in vitro that this hydrolytic process is highly enantioselective in that the catalytic efficiency of hCES1 is up to 6-fold higher for l-MPH than d-MPH. Furthermore, pharmacokinetic studies that have measured both isomers have consistently shown that the l-isomer is present at only 1-15% of the blood concentration of d-MPH. Additionally, the plasma half-life (t_{1/2}) of d-MPH is markedly longer than that of l-MPH.

Based on these observations, the present invention permits one to establish a drug metabolism profile for each drug and CES1 or variants thereof. By examining the CES1 gene or protein of the subject involved, one can then predict which drugs will be effective in the subject (if at all), and at which doses.

### Kits

The invention also provides kits for genotyping any one or more of a CES1 isoform (CES1A1, CES1A2, and CES1A3). Such kits are useful for the diagnosis of a sequence alteration in CES1 relative to wild-type CES1 in a biological sample obtained from a subject. Alternatively, the invention provides for selecting a drug treatment regimen or adjusting a dosage. In various embodiments, the kit includes at least one primer pair that identifies a CES1 nucleic acid sequence (e.g., CES1A1), together with instructions for using the primers to genotype in a biological sample. In additional embodiments, the kit also includes instructions for selecting an appropriate therapy for a subject, monitoring drug therapy in a subject, identifying a subject as responsive to drug therapy, or identifying a subject as sensitive to a drug. Advantageously, such testing is carried out prior to drug administration or after an adverse event associated with drug administration. Preferably, the primers are provided in combination with a thermostable DNA polymerase capable of long-range PCR amplification (e.g., a high density array). In yet another embodiment, the kit further comprises a pair of primers capable of binding to and amplifying a reference sequence. In yet other embodiments, the kit comprises a sterile container which contains the primers; such containers can be boxes, ampules, bottles, vials, tubes, bags, pouches, blister-packs, or other suitable container form known in the art. Such containers can be made of plastic, glass, laminated paper, metal foil, or other materials suitable for holding nucleic acids.

The instructions will generally include information about the use of the compositions of the invention in genotyping a CES1 gene isoform. In particular embodiments, the genotype identifies or characterizes a subject as having altered drug metabolism. In other embodiments, the instructions include at least one of the following: descriptions of the primer, methods for using the enclosed materials for the identification of a subject having altered drug metabolism; precautions; warnings; indications; clinical or research studies; and/or references. The instructions may be printed directly on the container (when present), or as a label applied to the container, or as a separate sheet, pamphlet, card, or folder supplied in or with the container.

In various other embodiments, the kit includes reagents or components for genotyping CES1 in combination with reagents or components for the detection of a single nucleotide polymorphism (SNP) or variant of a gene encoding an enzyme involved in drug metabolism (e.g., Cytochrome P450 2D6, Cytochrome P450 2C19) or drug transporter (e.g., an ATP-binding cassette (ABC) transporters). The kits which contain reagents and components for determining a CES1 genotype and for detecting variants in enzyme and transporters involved in drug metabolism, are useful for guiding disease specific pharmacotherapies. For example, in the treatment of congestive heart failure (CHF), one or more drugs, including ACE inhibitors, the ionotropic drug digoxin, and beta-blockers may be prescribed depending on their predicted efficacy in a patient. The patient is evaluated for CES1 expression or catalytic activity to predict the responsiveness of the patient to an ACE inhibitor that is a CES1 substrate. Additionally, the patient is evaluated for one or more of P-glycoprotein SNPs, which predict the responsiveness of the patient to digoxin or CYP450 2D6 SNPs, which predict the responsiveness of the patient to beta-blockers (CYP450 2D6 SNPs). Such kits may contain one or more genomic tests of enzymes or drug transporters documented to have important SNPs. SNPs may be evaluated using a disease targeted panel of tests (e.g., a microarray). Such panels include commercially available microarrays for detecting one or more SNPs (e.g., AmpliChip® CYP450 Test; Roche). In other embodiments, the kit includes instructions for selecting one or more treatments based on the results of genotyping CES1 and detecting one or more genetic variants in an enzyme involved in drug metabolism or drug transporter. Thus, testing performed on a patient using the kits of the invention may guide treatment selection specifically tailored to the individual.

The following examples are offered by way of illustration, not by way of limitation. While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims along with their full scope of equivalents.

It should be appreciated that the invention should not be construed to be limited to the examples that are now described; rather, the invention should be construed to include any and all applications provided herein and all equivalent variations within the skill of the ordinary artisan.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

### Genotyping of CES1 Isoforms Identifies the Presence of a Variant Allele in CES1A1 Linked to Reduced Drug Metabolism

The majority of hCES1 activity in the liver is due to the expression of the CES1A gene. When a variant allele linked to altered drug metabolism is present in CES1A1, it has a greater effect on altering drug metabolism than when the variant allele is present in CES1A2 or CES1A3 isoforms. CES1A1 is identical to CES1A2 except in the promoter region and exon 1 (Fukami et al., 2008). Current Taqman®-based methods for detecting CES1 variants are incapable of distinguishing variants in specific CES1 isoforms due to the high similarity of their DNA sequences. Therefore, genotyping of CES1 isoforms was used to distinguish in which CES1 isoform a variant allele was present in an individual having reduced drug metabolism.

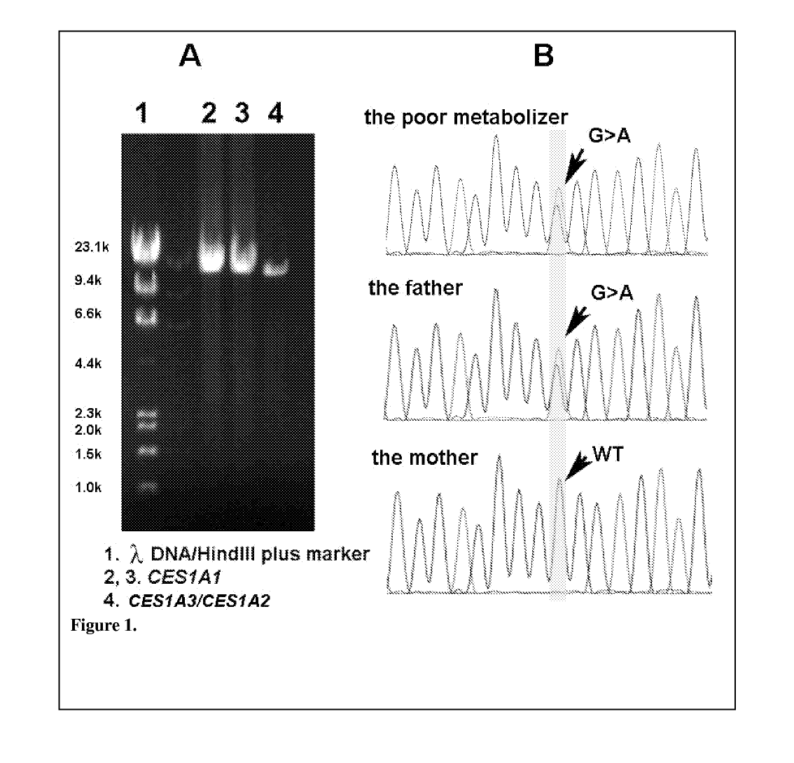

To develop a novel specific genotyping assay for CES1A and CES1A3/CES1A2 genes, a long-range PCR approach was used. Two sets of primers were designed in order to specifically amplify the fragments from CES1A1 and CES1A3/CES1A2 genes. A commercially available thermostable DNA polymerase was used (TaKaRa LA Taq™). The primer sequences and PCR reaction conditions are shown in Table 3. Both PCR reactions yielded ˜14 kb amplification products, which span from exon 1 to exon 6 of CES1A1 and CES1A3/CES1A2 genes (FIG. 1A). Bidirectional sequencing of the long-range PCR products revealed that specific amplification of CES1A1 and CES1A3/CES1A2 genes was achieved under the experimental conditions summarized in Table 3. The long-range PCR products can be used as the templates for CES1A1 and CES1A3/CES1A2 genotyping (e.g., via direct DNA sequencing or Taqman® assay).

TABLE 3
Specific long-range PCR of CES1A1 and CES1A3/CES1A2 genes
Primers for the long-range PCR of CES1A1 and CES1A3/CES1A2 genes

| Genes | Primers |
|---|---|
| CES1A1 | Forward: 5′-TTC CAC GAT GTG CCG TGC CTT TA-3′ |
| | Reverse: 5′-GGC ACA TAG GAG GAG TGT GGT CAC A-3′ |
| CES1A3/CES1A2 | Forward: 5′-TTC CAG GAT GTG GCT CCCTGC TCT TG-3′ |
| | Reverse: 5′-GGC ACA TAG GAG GAG TGT GGT CAC A-3′ |

PCR mixture composition

| | |
|---|---|
| 10×LA PCR buffer II (Mg²⁺ plus) | 5 μl |
| dNTP mixture (2.5 mM each) | 8 μl |
| genomic DNA | 200 ng |
| TaKaRa LA Taq (5 units/μl) | 1 μl |
| Forward primer | Final concentration 0.2 μM |
| Reverse primer | Final concentration 0.2 μM |
| | Add water up to 50 μl |

PCR reaction conditions

| | |
|---|---|
| 94° C. | 1 min |
| 94° C. | 30 sec 30× |
| 68° C. | 14 min |
| 72° C. | 10 min |
| 4° C. | ∞ |

To determine whether the novel CES1 SNP Gly143Glu (428G>A) allele was located in CES1A1 or CES1A3/CES1A2, genotyping using the novel assay was performed on DNA samples collected from a poor metabolizer (PM) and the biological parents. Sequencing of the CES1A1 PCR product from the poor metabolizer and the father demonstrated G and A peaks at position 428. In the mother, sequencing detected only an A peak at position 428, corresponding to the wild-type CES1A1 allele. The results demonstrated that the PM and the father were heterozygous Gly143Glu/wild-type in CES1A1, and the mother was homozygous wild-type in CES1A1 (FIG. 1B). Additional sequencing revealed that all three subjects were wild-type for CES1A2, but not CES1A3.

Thus, the data indicated that in poor metabolizers SNP Gly143Glu was located in CES1A1 and not in CES1A3/CES1A2. Because the majority of hCES1 in the liver is the product of CES1A1 gene, the genotyping results validate clinical findings that the variant Gly143Glu has a profound impact on hCES1 functions.

### Example 2

### Genotyping of CES1A1 and CES1A3/CES1A2 Distinguishes Individuals Homozygous or Heterozygous for CES1A1 Variant Alleles

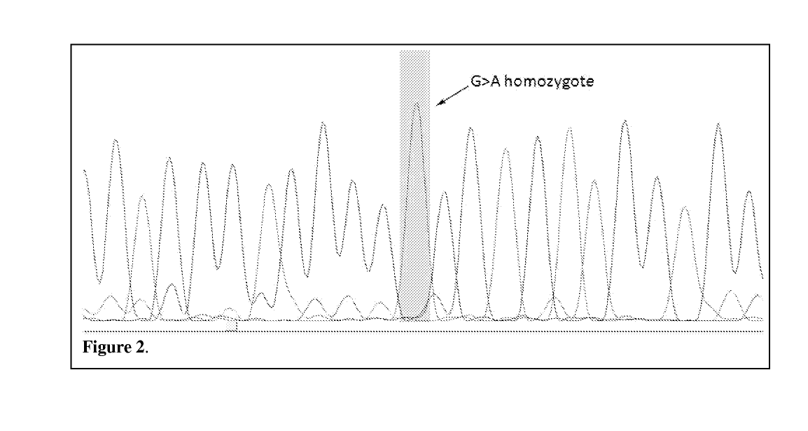

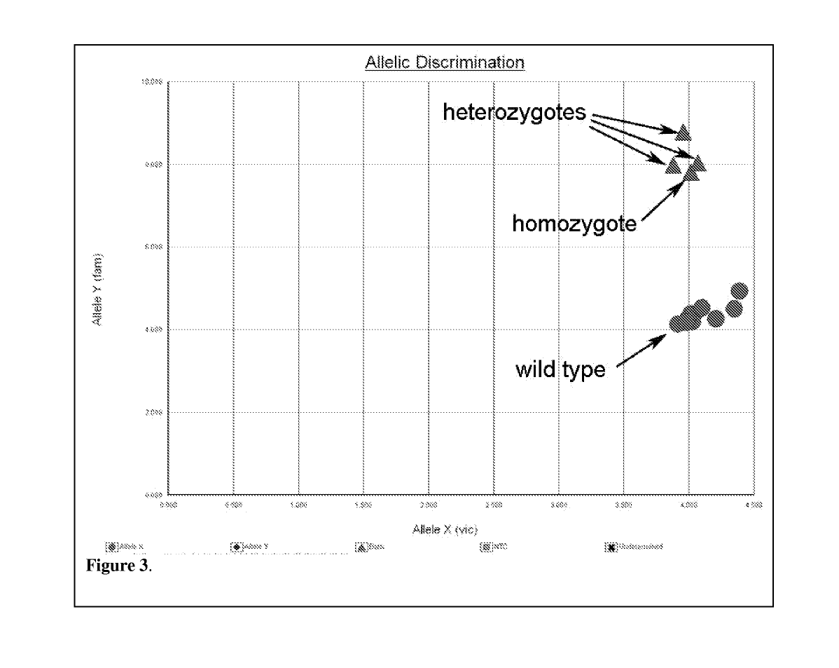

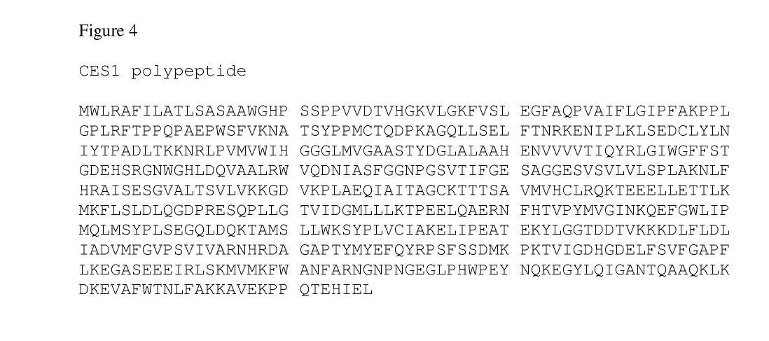

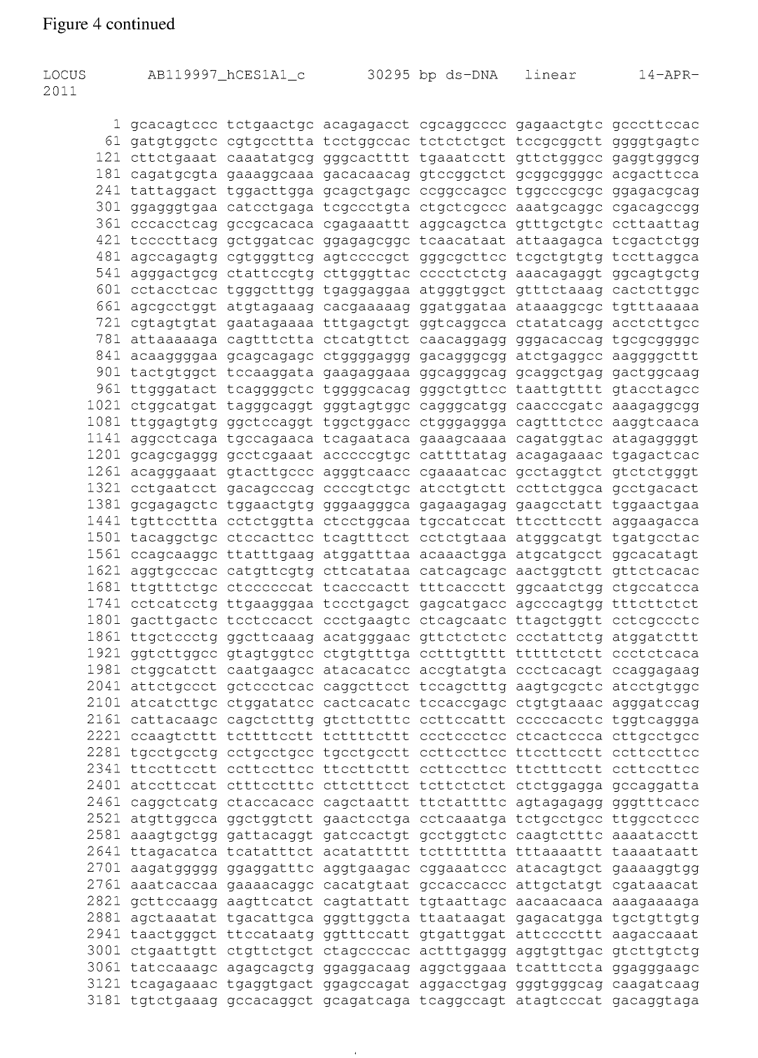

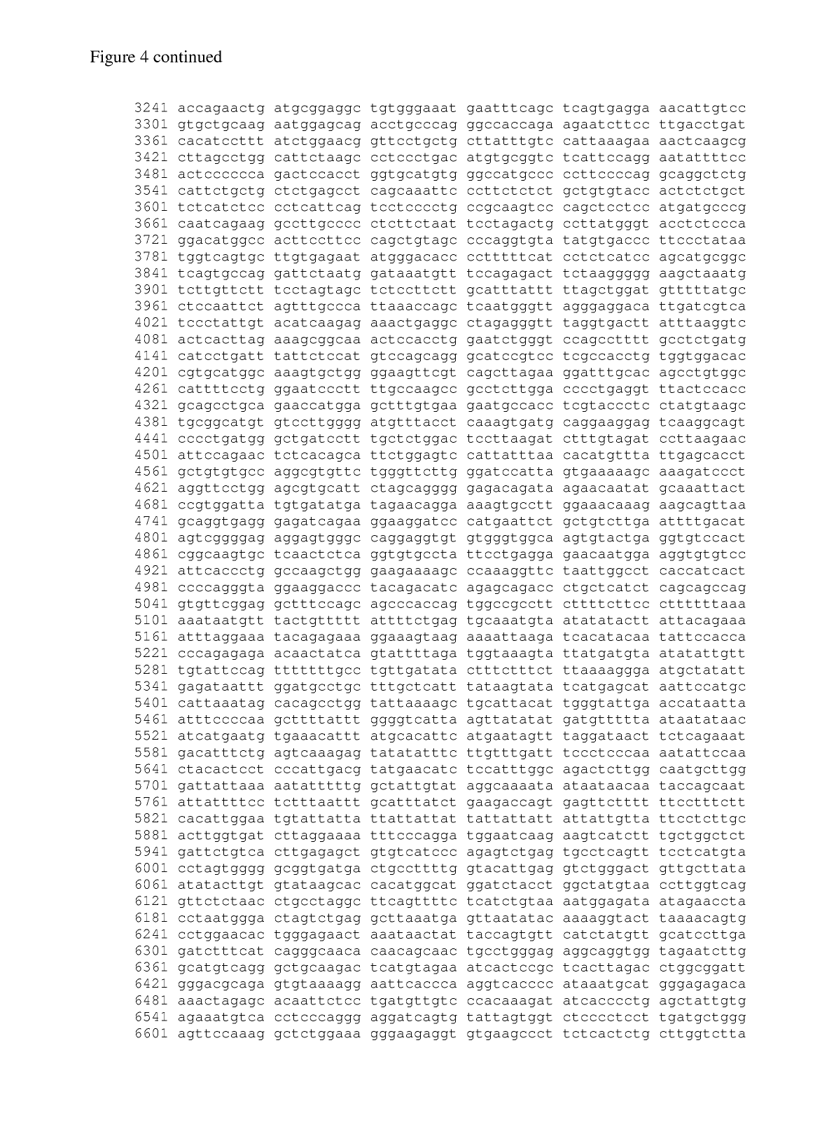

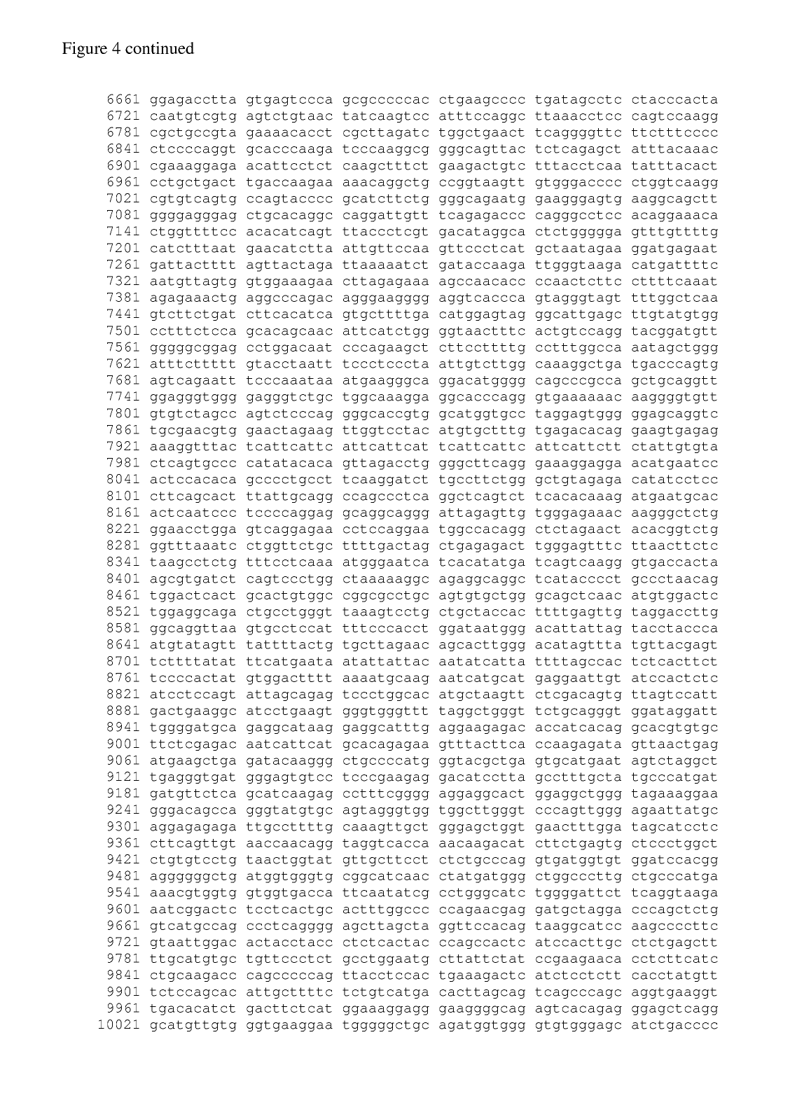

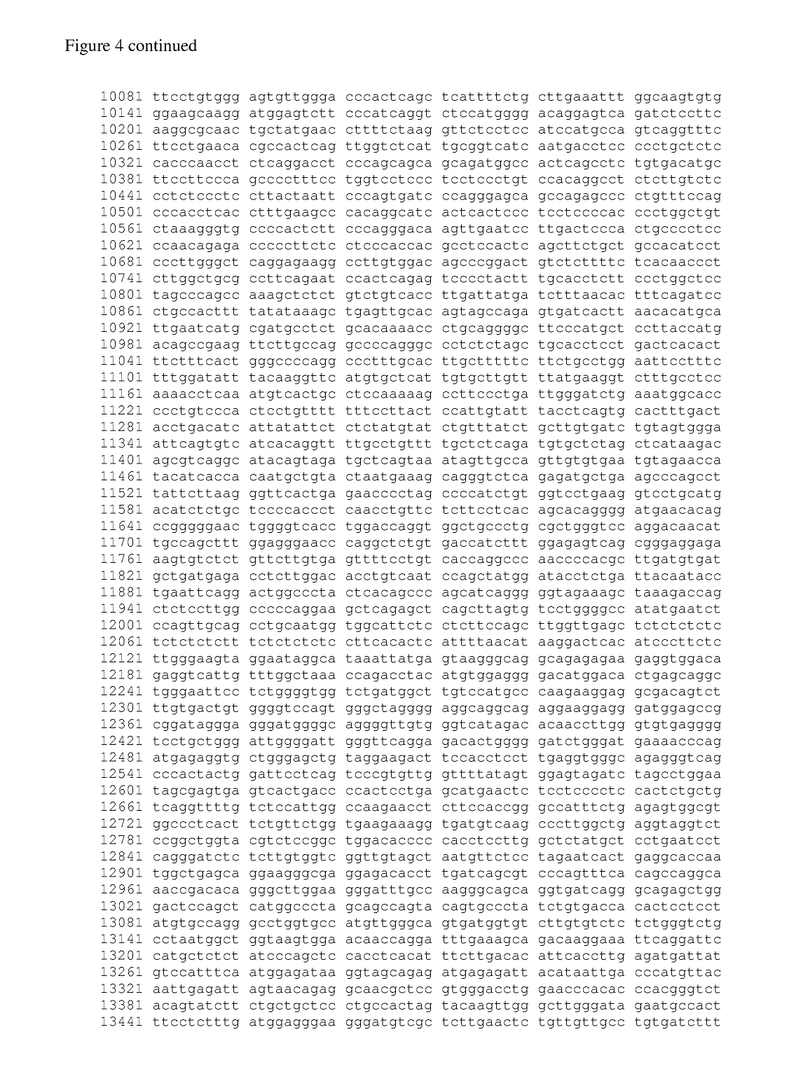

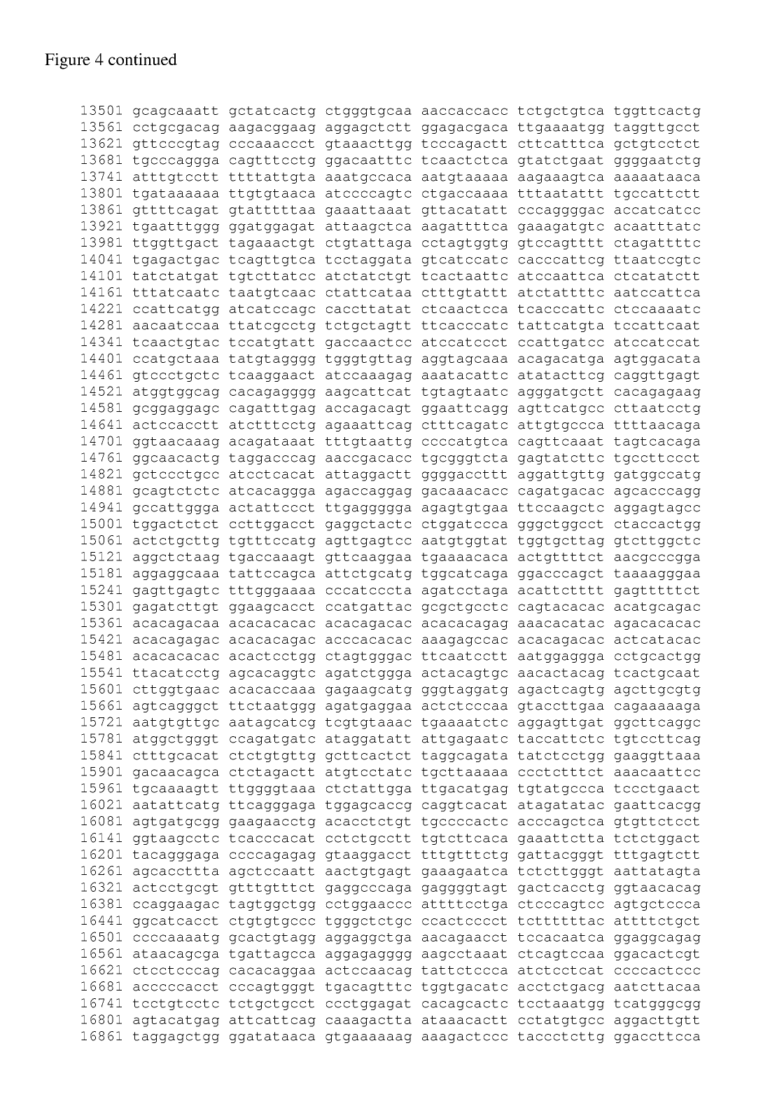

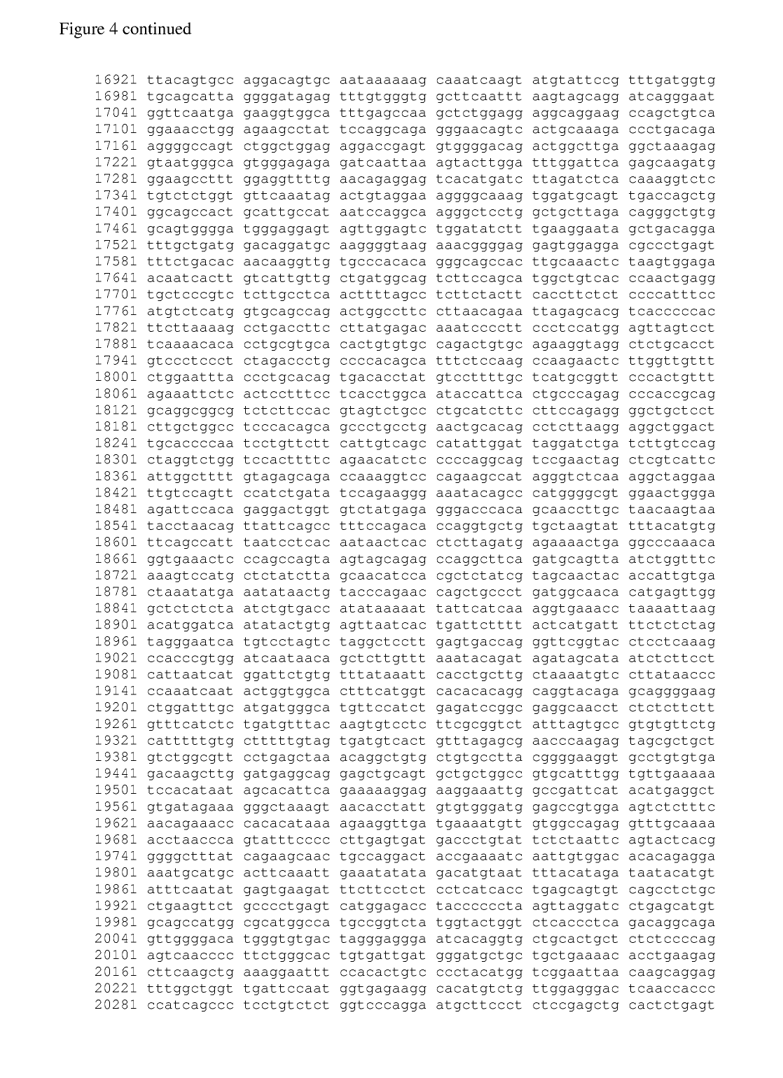

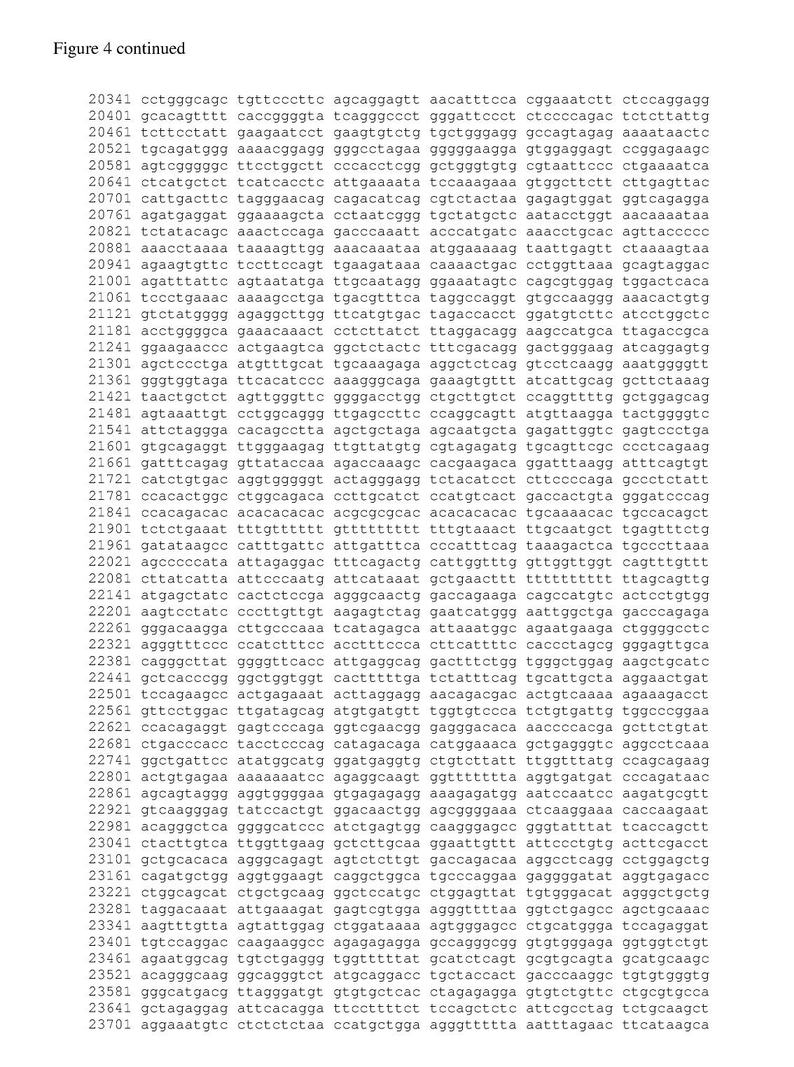

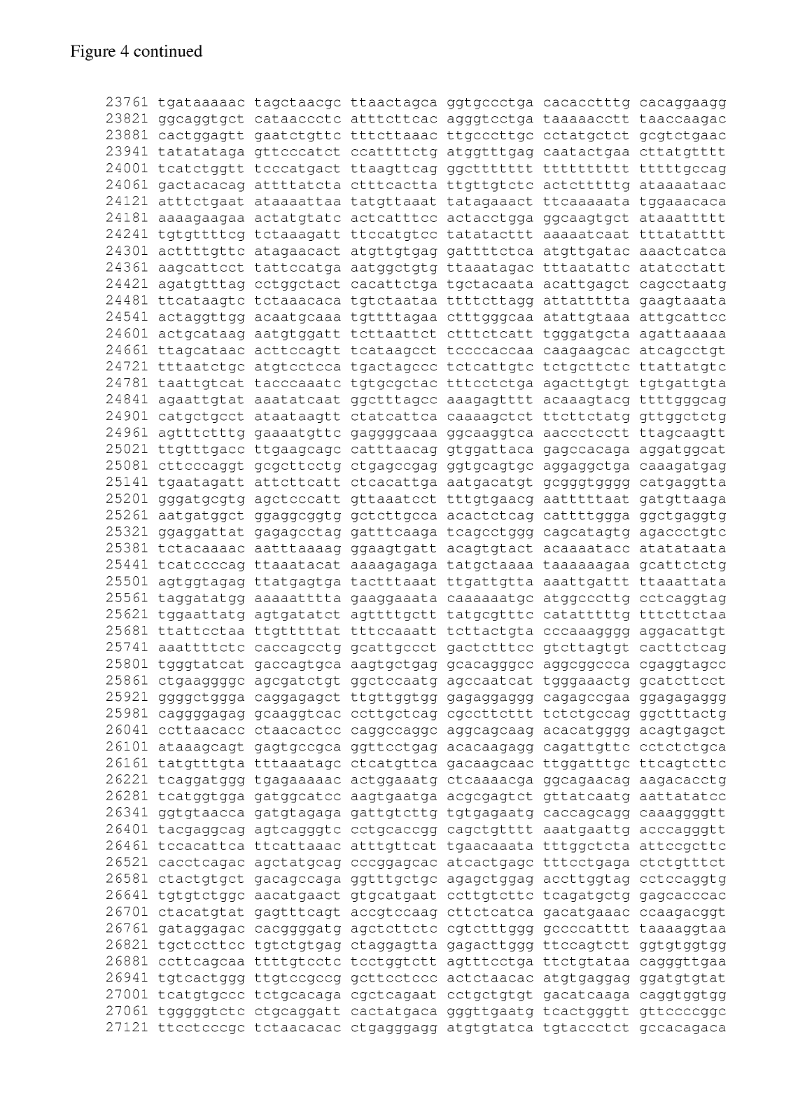

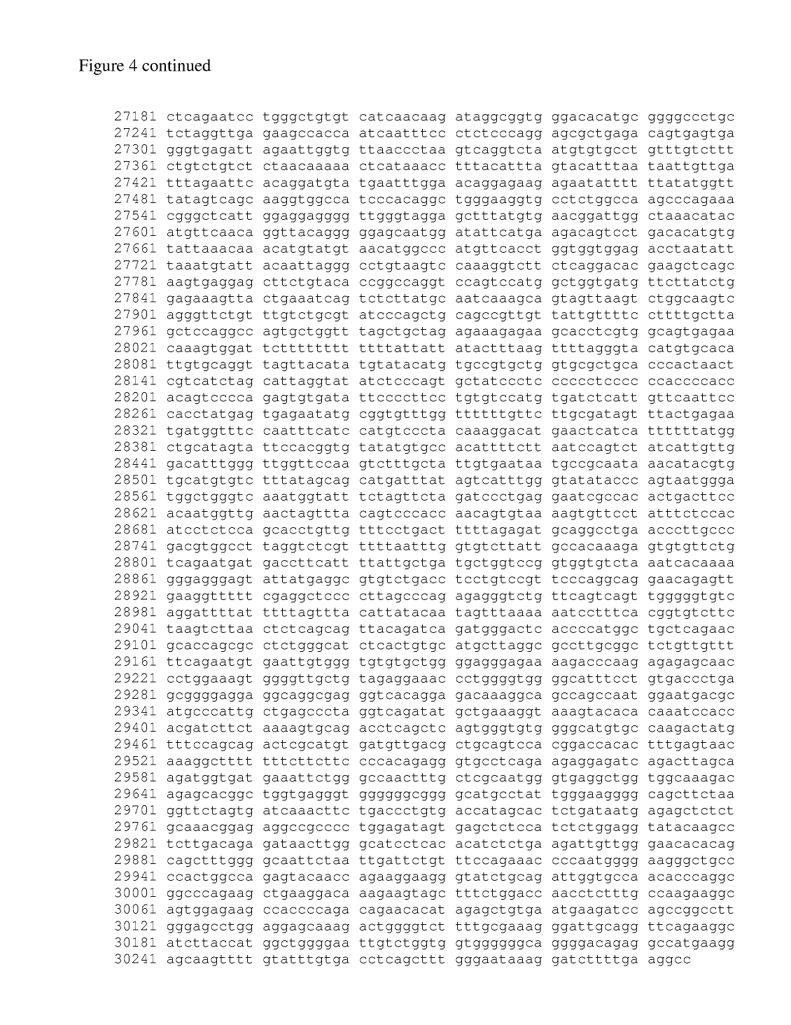

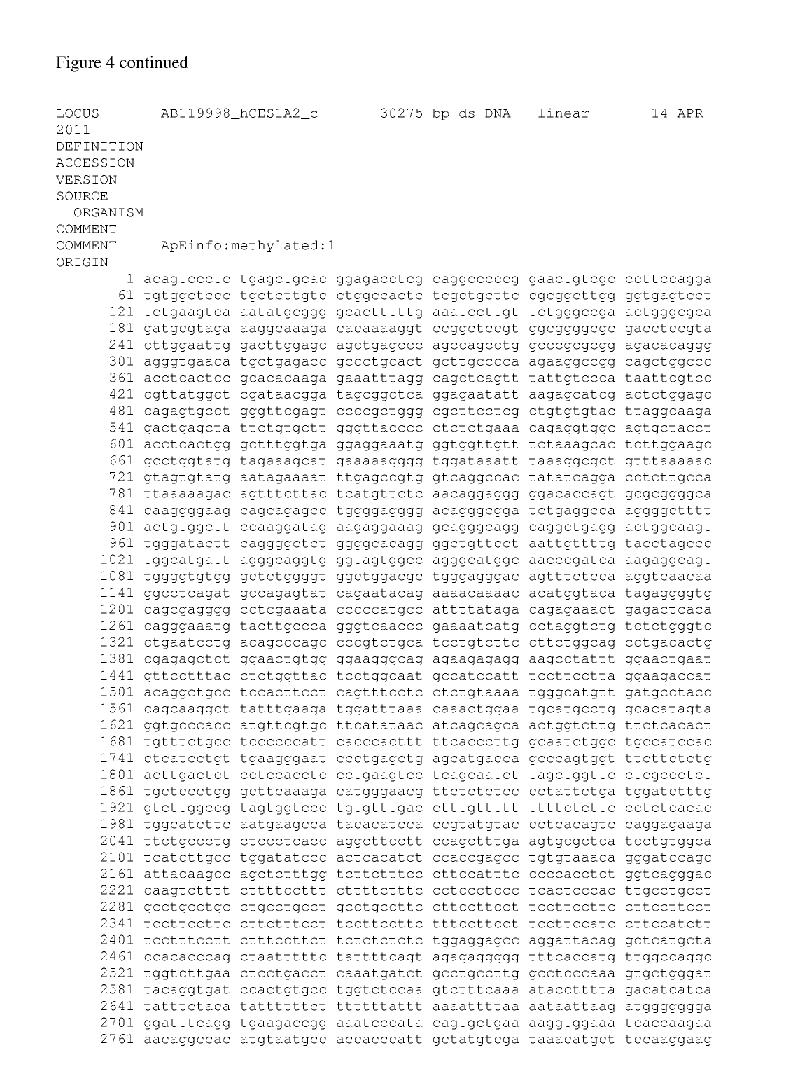

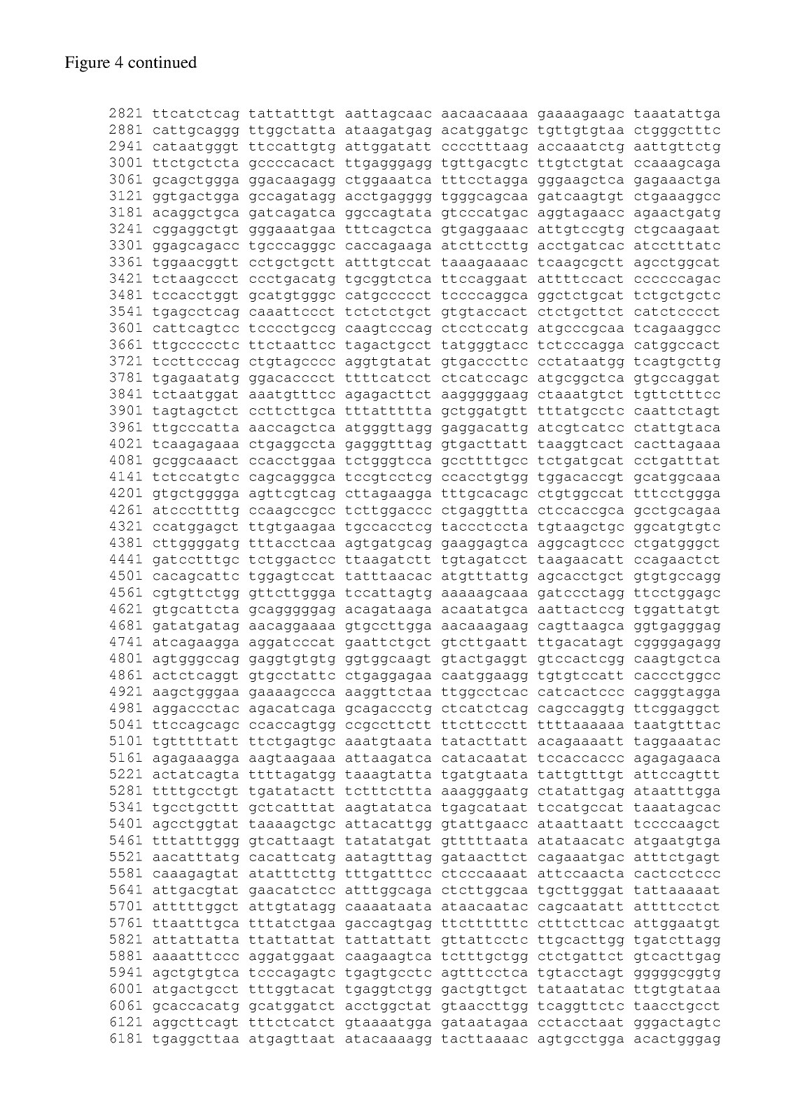

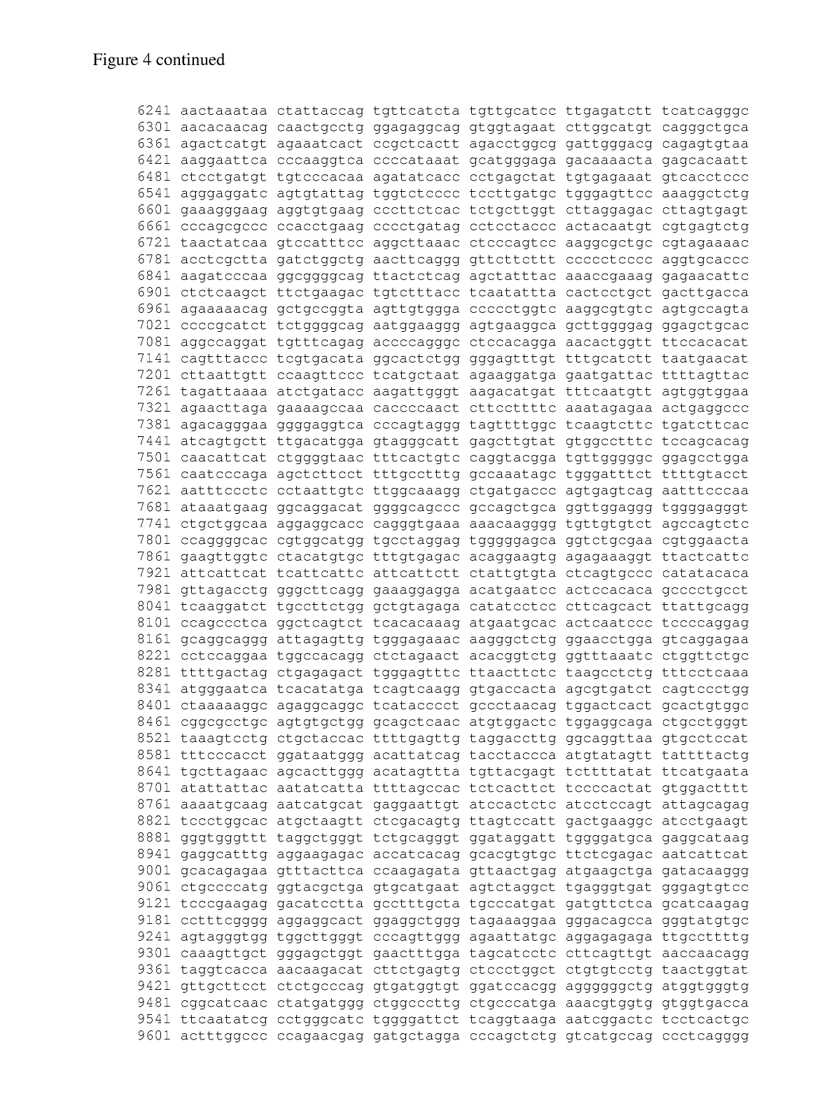

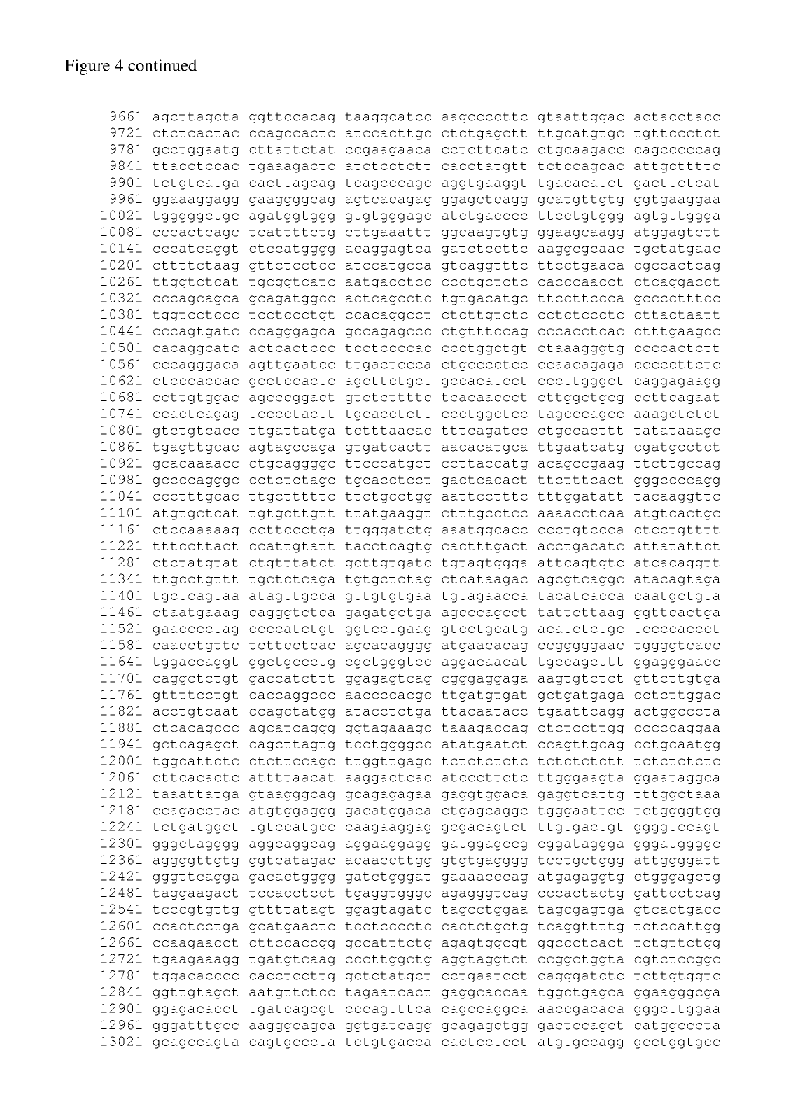

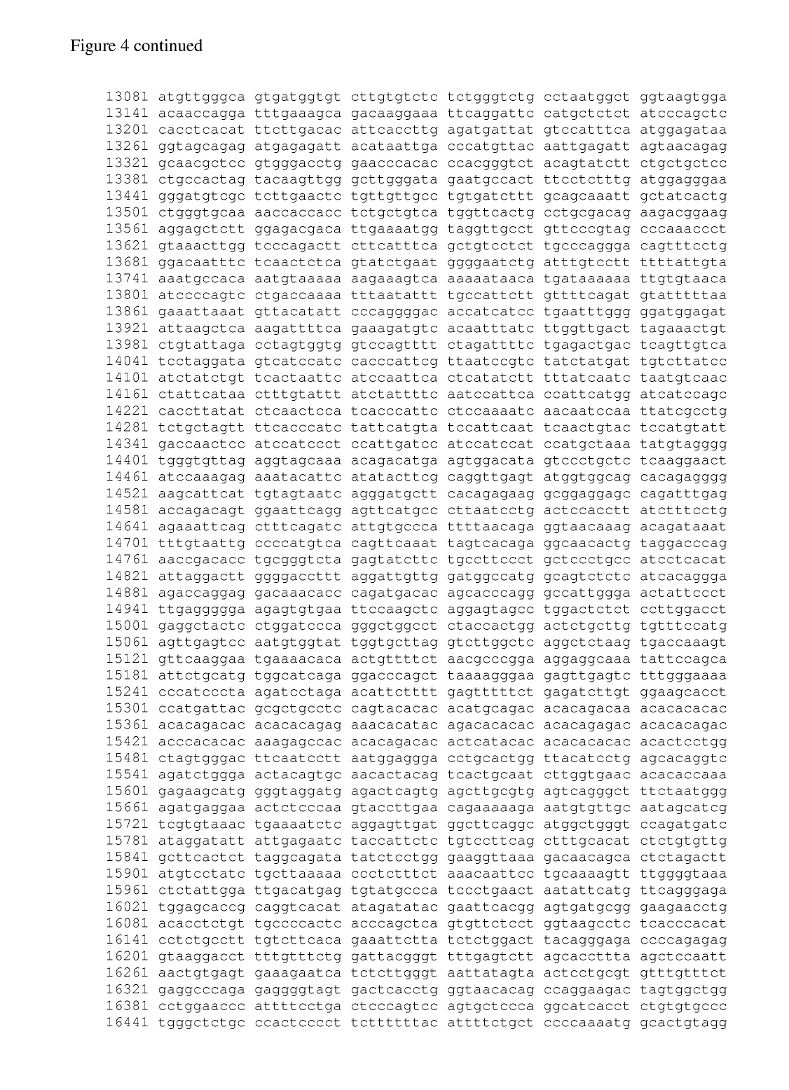

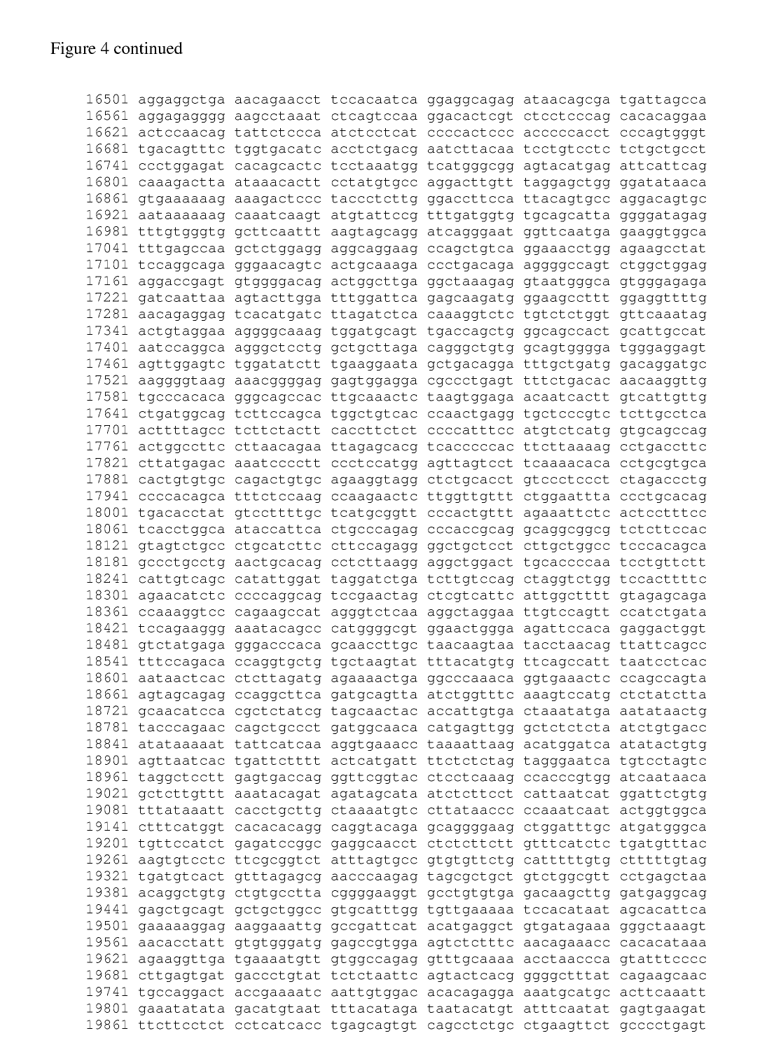

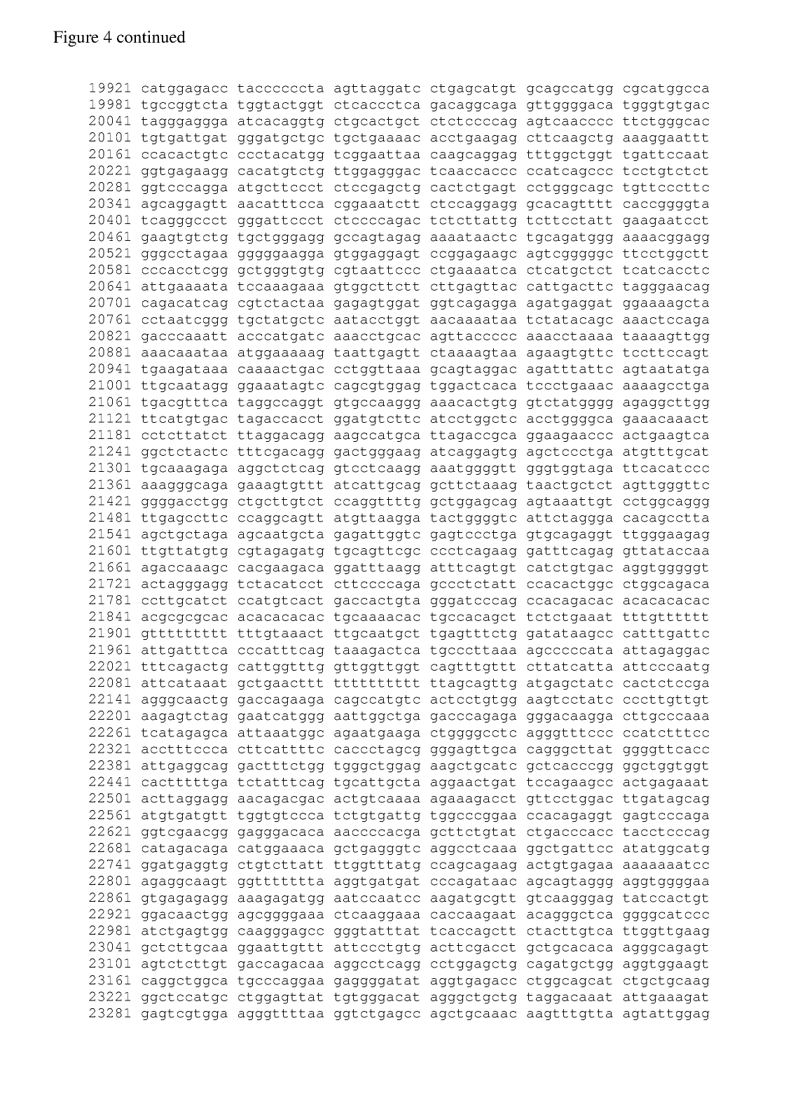

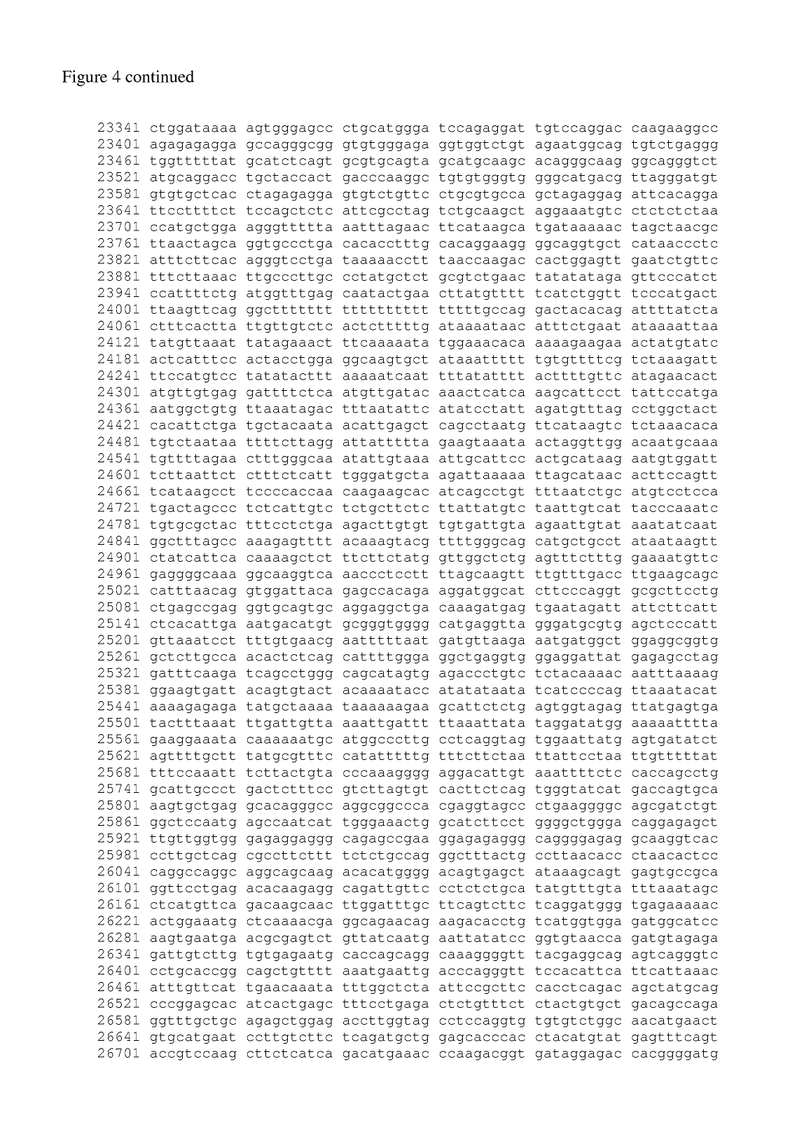

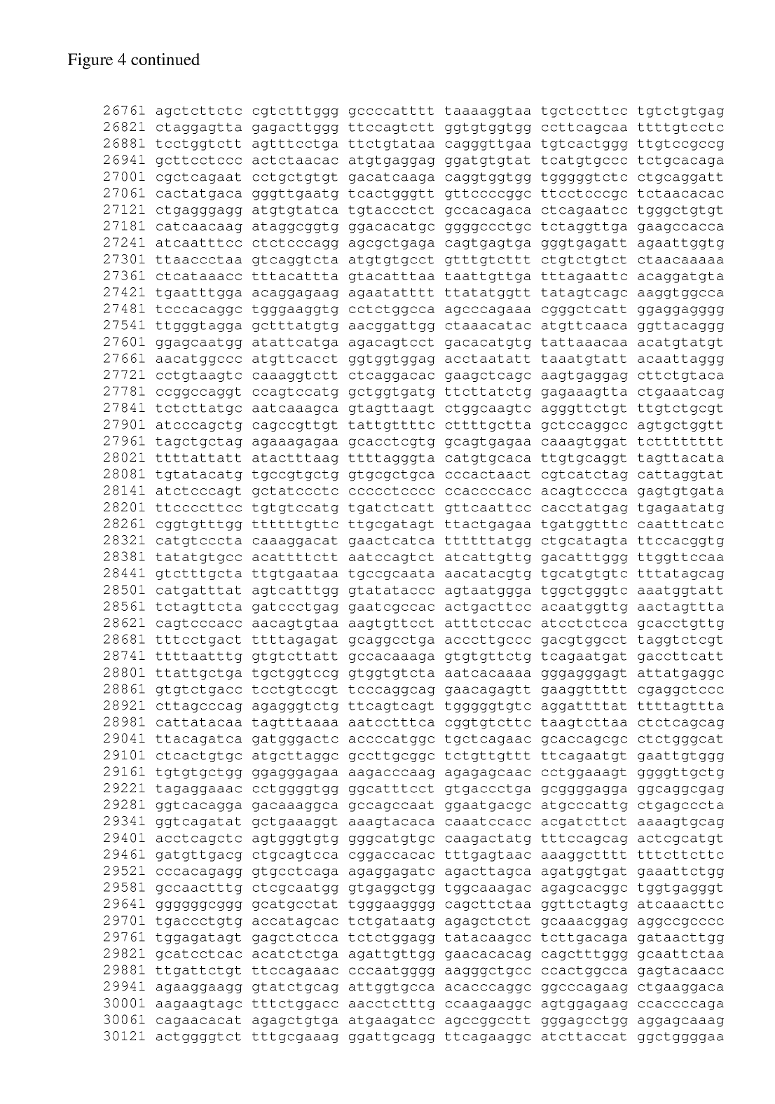

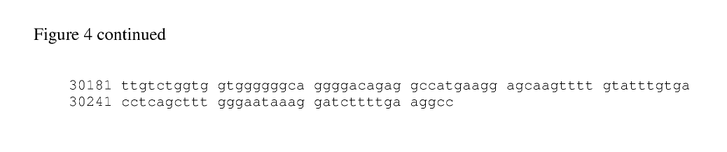

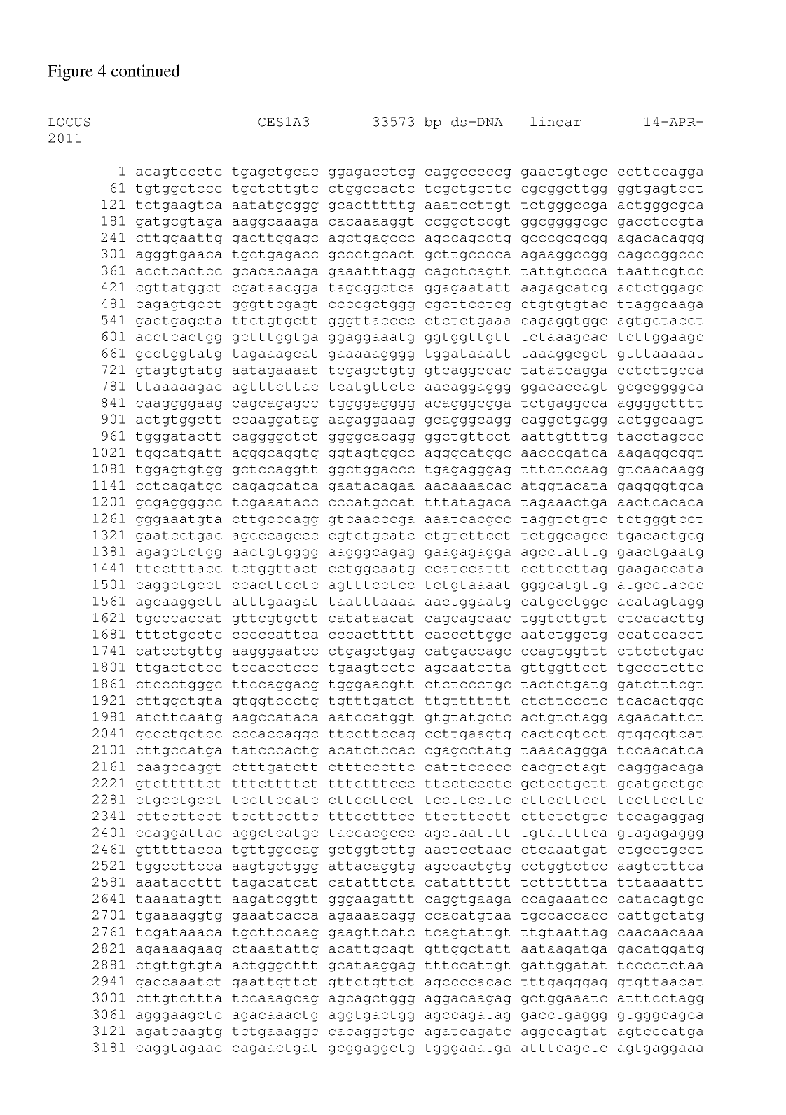

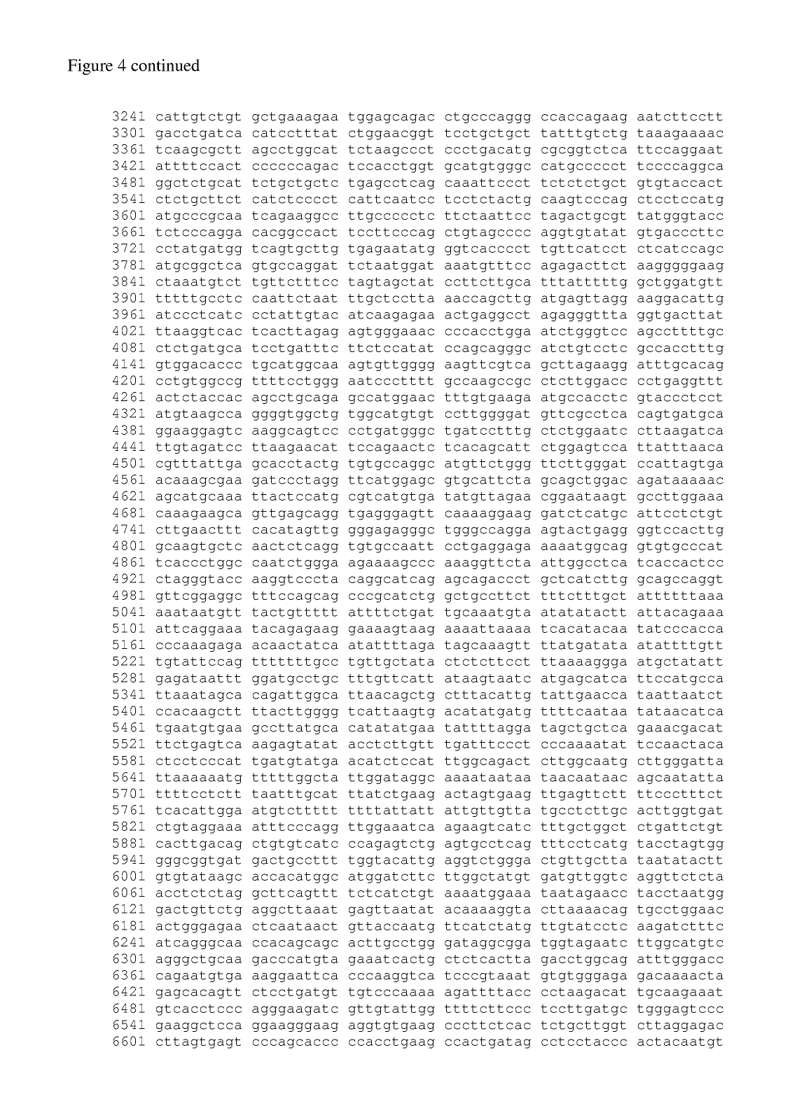

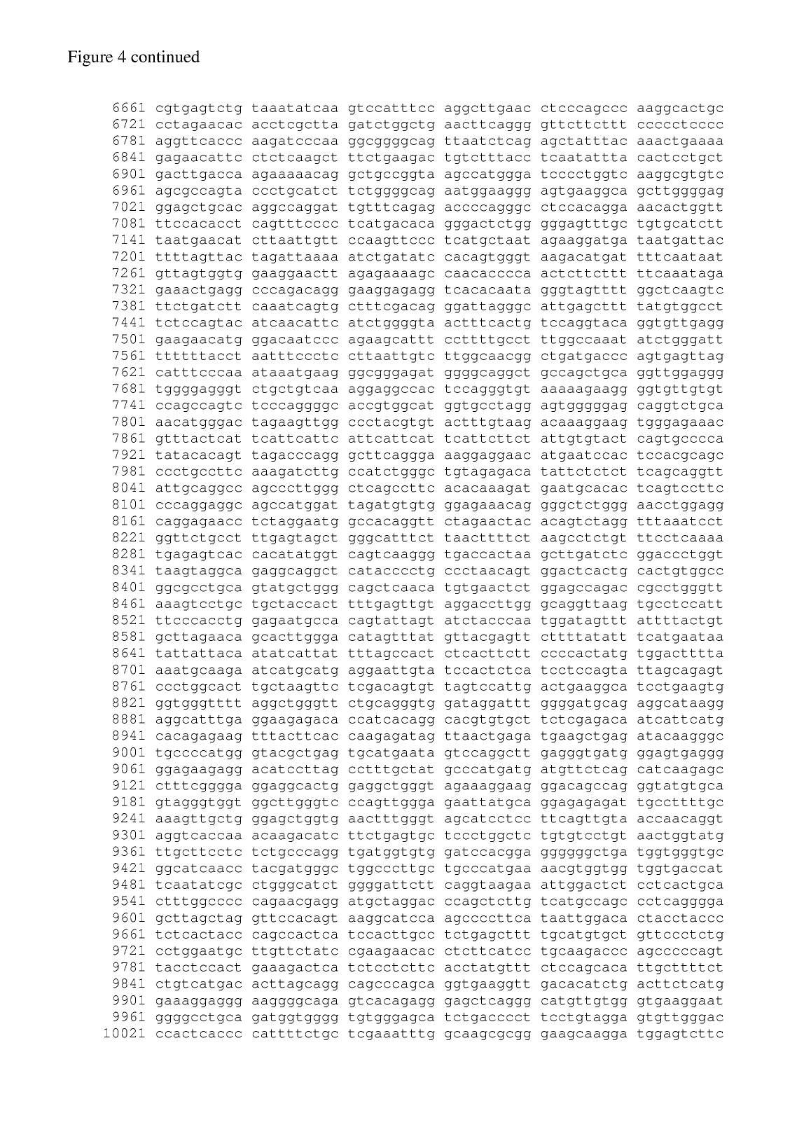

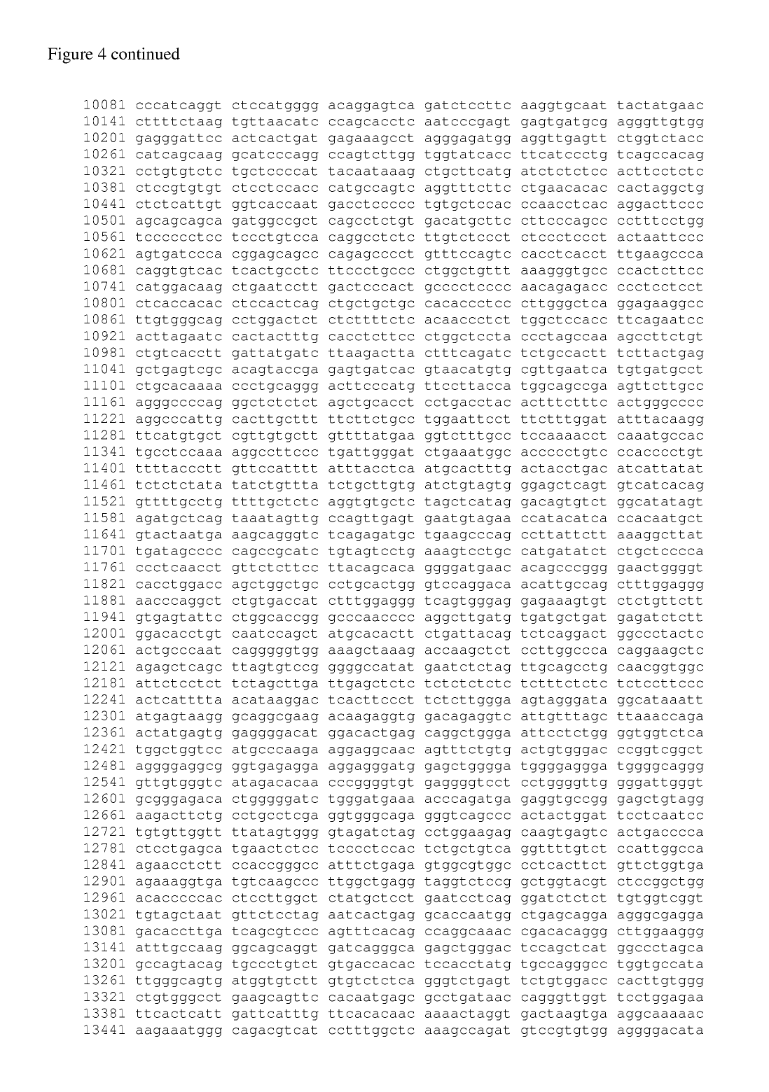

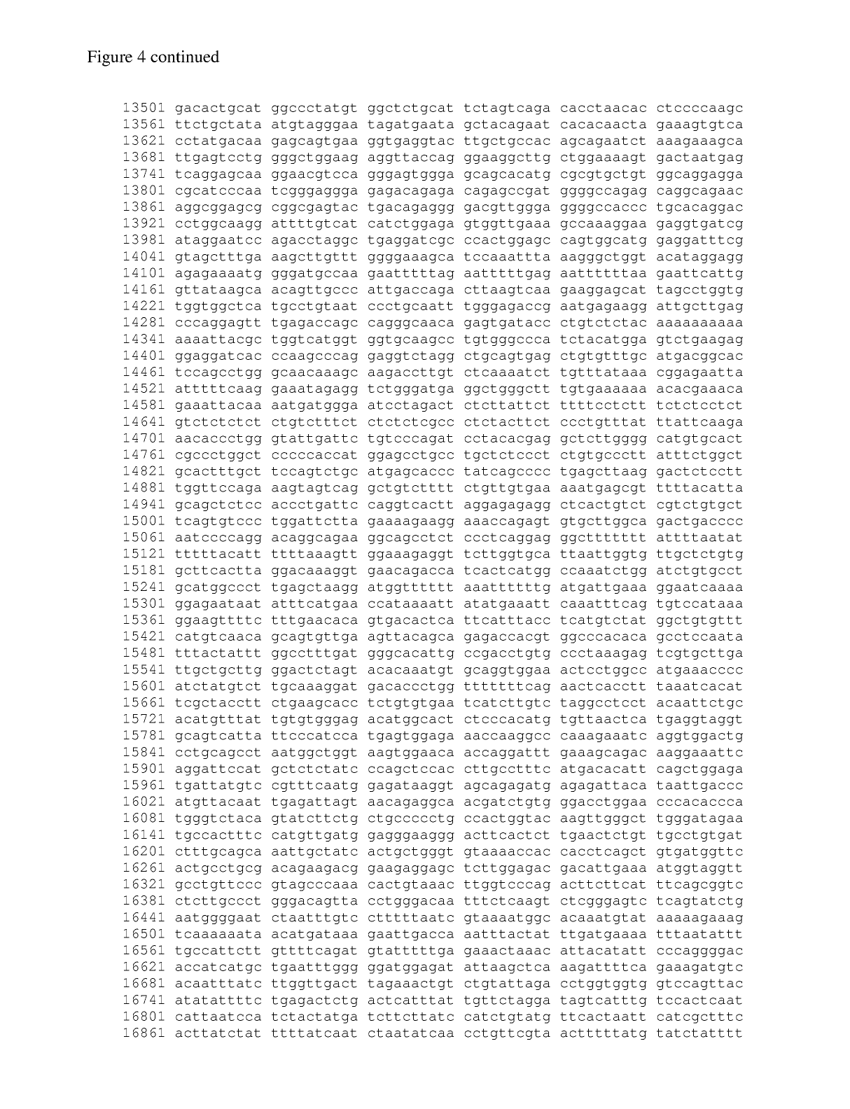

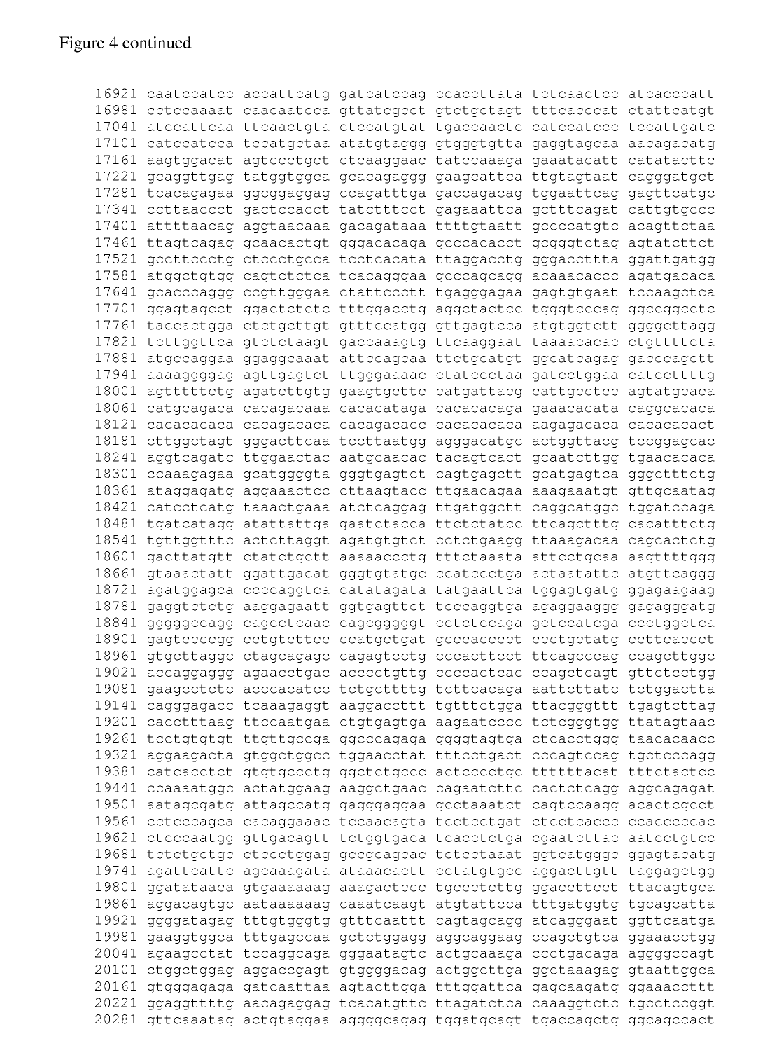

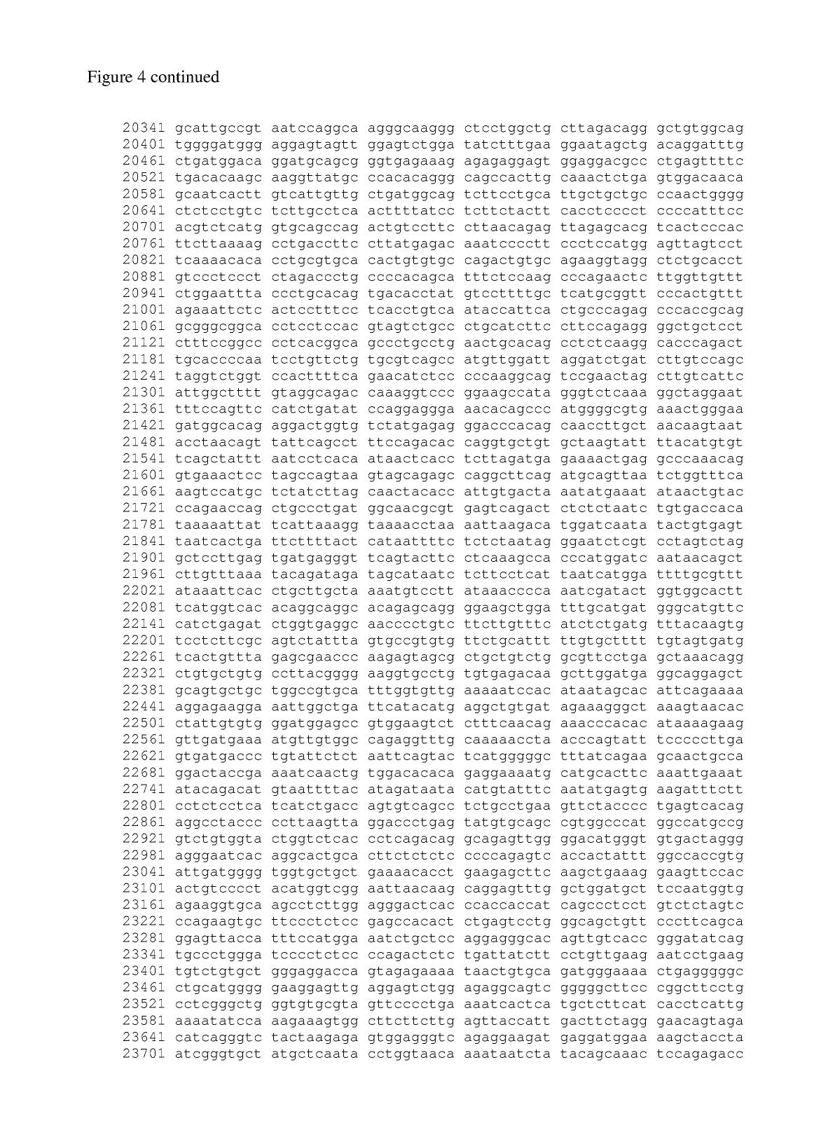

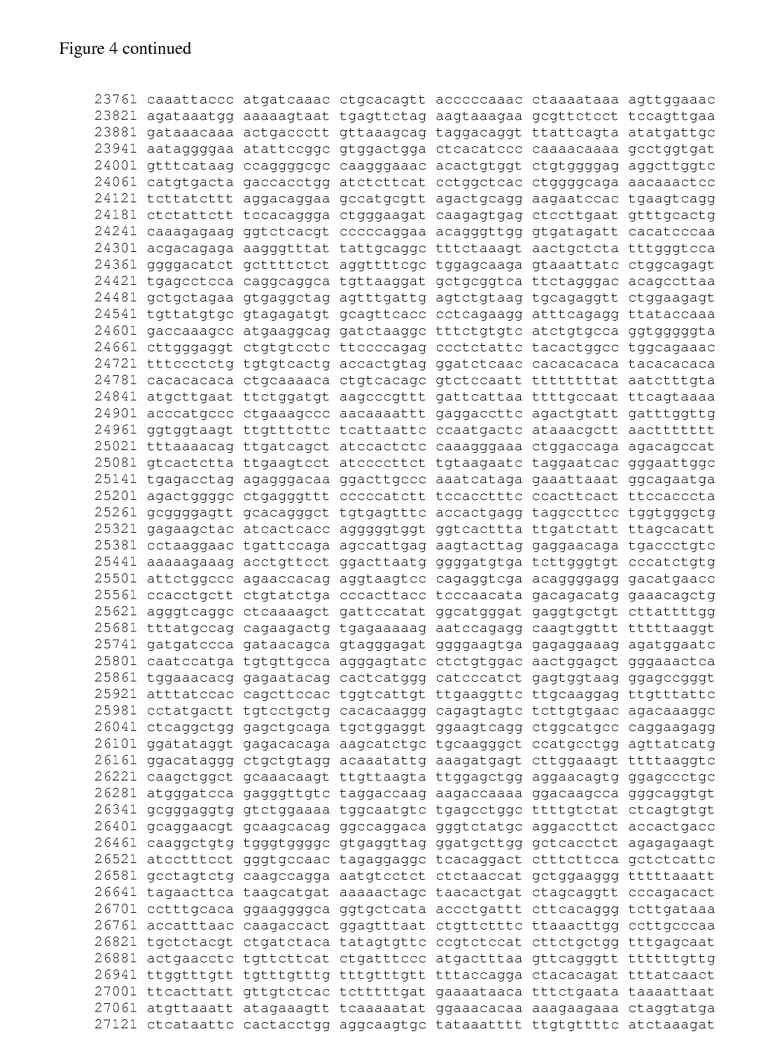

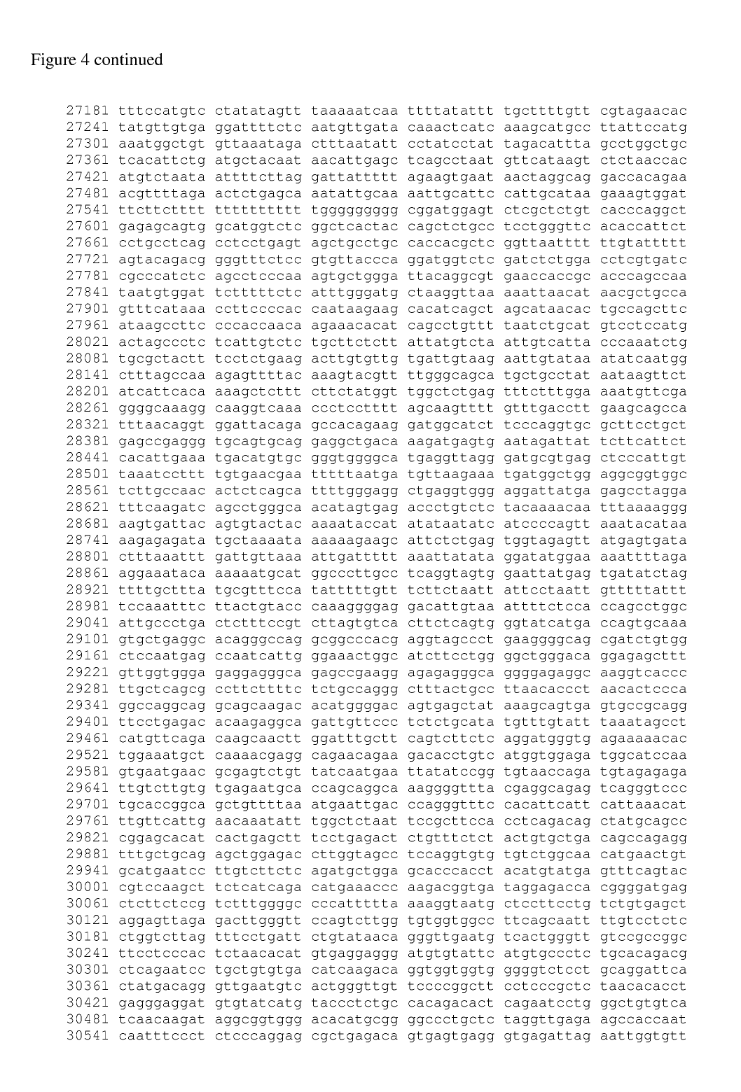

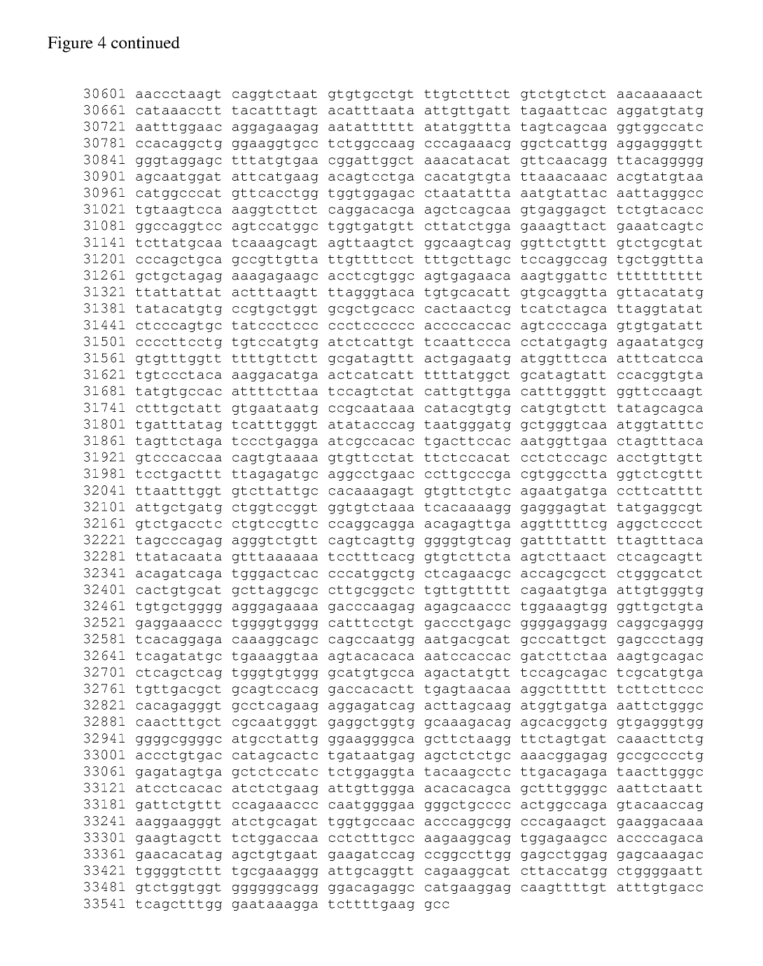

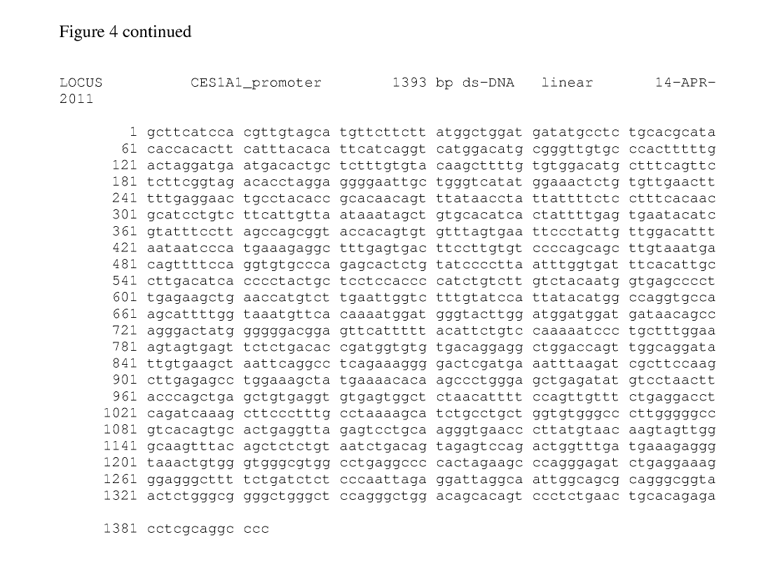

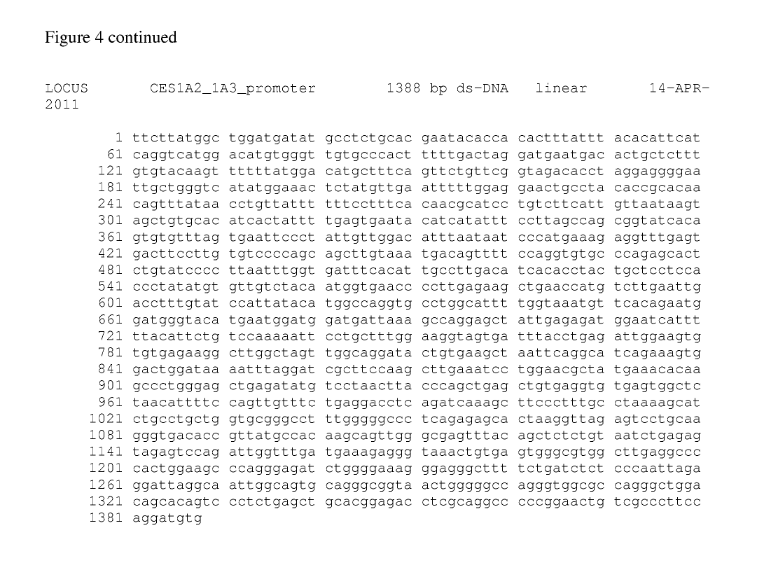

The CES1 genotyping assay was used to genotype the CES1A1 variant Gly143Glu in 107 saliva DNA samples collected from ADHD patients being treated with MPH. Among them, one homozygote (FIG. 2) and three heterozygotes were indentified, while the others were wild-type (WT). This is the first instance that an individual homozygous Gly143Glu at CES1A1 has been reported. By comparison, a previously developed Taqman® detection assay was employed to examine the DNA samples from selected subjects including the Gly143Glu homozygote, three heterozygotes, and ten randomly selected WT. The Taqman® assay was performed as previously described (Zhu et al., 2008). The Taqman® assay was not able to distinguish the homozygote from the other three heterozygotes, but grouped the homozygote with the Gly143Glu heterozygotes (FIG. 3). The results provided direct evidence that non-isoform specific assays that do not discriminate among CES1 isoforms, are not suitable for genotyping CES1 genetic polymorphisms. In fact, screening of 1607 total subjects by non-isoform specific CES1 assays in 3 recently published independent studies did not identify any Gly143Glu homozygotes (Zhu et al., 2008; Nemoda et al., 2009; Walter Soria et al., 2010). This result is likely due to the inability of these assays to distinguishing between CES1A1 and CES1A2, and CES1A3 genes. The discriminative CES1 isoform genotyping assay described in the study is preferable for CES1 pharmacogenetic studies in both clinical and basic research settings.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

This application may be related to U.S. patent application Ser. No. 12/663,644, which is the U.S. national phase application, pursuant to 35 U.S.C. §371, of International Patent Application No.: PCT/US2008/0066280, filed Jun. 9, 2008, which claims the benefit of U.S. Provisional Application Ser. Nos. 60/942,818, 61/051,680, and 61/053,524, the disclosures of which are hereby incorporated herein in their entireties by reference.

All patents and publications mentioned in this specification are herein incorporated by reference to the same extent as if each independent patent and publication was specifically and individually indicated to be incorporated by reference.

### REFERENCES

The following documents are cited herein.
Bencharit S, Edwards C C, Morton C L, Howard-Williams E L, Kuhn P, Potter P M and Redinbo M R (2006) Multisite promiscuity in the processing of endogenous substrates by human carboxylesterase 1. J Mol Biol 363:201-214.
Bencharit S, Morton C L, Xue Y, Potter P M and Redinbo M R (2003) Structural basis of heroin and cocaine metabolism by a promiscuous human drug-processing enzyme. Nat Struct Biol 10:349-356.
Brzezinski M R, Spink B J, Dean R A, Berkman C E, Cashman J R and Bosron W F (1997) Human liver carboxylesterase hCE-1: binding specificity for cocaine, heroin, and their metabolites and analogs. Drug Metab Dispos 25:1089-1096.
Fleming C D, Bencharit S, Edwards C C, et al. Structural Insights into Drug Processing by Human Carboxylesterase 1: Tamoxifen, Mevastatin, and Inhibition by Benzil. Journal of Molecular Biology 2005; 352: 165-177.
Fukami T, Nakajima M, Maruichi T, Takahashi S, Takamiya M, Aoki Y, McLeod H L and Yokoi T (2008) Structure and characterization of human carboxylesterase 1A1, 1A2, and 1A3 genes. Pharmacogenet Genomics 18:911-920.
Geshi E, Kimura T, Yoshimura M, Suzuki H, Koba S, Sakai T, Saito T, Koga A, Muramatsu M and Katagiri T (2005) A single nucleotide polymorphism in the carboxylesterase gene is associated with the responsiveness to imidapril medication and the promoter activity. Hypertens Res 28:719-725.
Hosokawa M (2010) Are non-human primates useful experimental animals for pre-clinical study?Drug Metab Pharmacokinet 25:221-222.
Hosokawa M, Furihata T, Yaginuma Y, Yamamoto N, Koyano N, Fujii A, Nagahara Y, Satoh T and Chiba K (2007) Genomic structure and transcriptional regulation of the rat, mouse, and human carboxylesterase genes. Drug Metab Rev 39:1-15.
Hosokawa M, Furihata T, Yaginuma Y, Yamamoto N, Watanabe N, Tsukada E, Ohhata Y, Kobayashi K, Satoh T and Chiba K (2008) Structural organization and characterization of the regulatory element of the human carboxylesterase (CES1A1 and CES1A2) genes. Drug Metab Pharmacokinet 23:73-84.
Imai T (2006) Human carboxylesterase isozymes: catalytic properties and rational drug design. Drug Metab Pharmacokinet 21:173-185.
Imai T, Taketani M, Shii M, Hosokawa M and Chiba K (2006) Substrate specificity of carboxylesterase isozymes and their contribution to hydrolase activity in human liver and small intestine. Drug Metab Dispos 34:1734-1741.
Masson P, Froment M-T, Gillon E, et al. Hydrolysis of oxo- and thio-esters by human butyrylcholinesterase. Biochimica et Biophysica Acta (BBA)—Proteins & Proteomics 2007; 1774: 16-34.
Nemoda Z, Angyal N, Tarnok Z, Gadoros J and Sasvari-Szekely M (2009) Carboxylesterase 1 gene polymorphism and methylphenidate response in ADHD. Neuropharmacology 57:731-733.
Patrick K S, Straughn A B, Minhinnett R R, Yeatts S D, Herrin A E, DeVane C L, Malcolm R, Janis G C and Markowitz J S (2007) Influence of ethanol and gender on methylphenidate pharmacokinetics and pharmacodynamics. Clin Pharmacol Ther 81:346-353.
Ross M K and Crow J A (2007) Human carboxylesterases and their role in xenobiotic and endobiotic metabolism. J Biochem Mol Toxicol 21:187-196.
Satoh T and Hosokawa M (1998) The mammalian carboxylesterases: from molecules to functions. Annu Rev Pharmacol Toxicol 38:257-288.
Satoh T, Taylor P, Bosron W F, Sanghani S P, Hosokawa M and La Du B N (2002) Current progress on esterases: from molecular structure to function. Drug Metab Dispos 30:488-493.
Shi D, Yang J, Yang D, LeCluyse E L, Black C, You L, Akhlaghi F and Yan B (2006) Anti-influenza prodrug oseltamivir is activated by carboxylesterase human carboxylesterase 1, and the activation is inhibited by antiplatelet agent clopidogrel. J Pharmacol Exp Ther 319:1477-1484.
Sun Z, Murry D J, Sanghani S P, Davis W I, Kedishvili N Y, Zou Q, Hurley T D and Bosron W F (2004) Methylphenidate is stereoselectively hydrolyzed by human carboxylesterase CES1A1. J Pharmacol Exp Ther 310:469-476.
Walter Soria N, Belaus A, Galvan C, Ana Pasquali M, Velez P, Del Carmen Montes C and Beltramo D M (2010) A Simple Allele-Specific Polymerase Chain Reaction Method to Detect the Gly143Glu Polymorphism in the Human Carboxylesterase 1 Gene: Importance of Genotyping for Pharmacogenetic Treatment. Genet Test Mol Biomarkers.
Yamada S, Richardson K, Tang M, Halaschek-Wiener J, Cook V J, Fitzgerald J M, Elwood K, Marra F and Brooks-Wilson A (2010) Genetic variation in carboxylesterase genes and susceptibility to isoniazid-induced hepatotoxicity. Pharmacogenomics J.
Yang D, Pearce R E, Wang X, Gaedigk R, Wan Y J and Yan B (2009) Human carboxylesterases HCE1 and HCE2: ontogenic expression, inter-individual variability and differential hydrolysis of oseltamivir, aspirin, deltamethrin and permethrin. Biochem Pharmacol 77:238-247.
Yoshimura M, Kimura T, Ishii M, Ishii K, Matsuura T, Geshi E, Hosokawa M and Muramatsu M (2008) Functional polymorphisms in carboxylesterase1A2 (CES1A2) gene involves specific protein 1 (Sp1) binding sites. Biochem Biophys Res Commun 369:939-942.
Zhu H J, Appel D I, Jiang Y and Markowitz J S (2009a) Age- and sex-related expression and activity of carboxylesterase 1 and 2 in mouse and human liver. Drug Metab Dispos 37:1819-1825.
Zhu H J, Appel D I, Johnson J A, Chavin K D and Markowitz J S (2009b) Role of carboxylesterase 1 and impact of natural genetic variants on the hydrolysis of trandolapril. Biochem Pharmacol 77:1266-1272.
Zhu H J and Markowitz J S (2009) Activation of the antiviral prodrug oseltamivir is impaired by two newly identified carboxylesterase 1 variants. Drug Metab Dispos 37:264-267.
Zhu H J, Patrick K S, Yuan H J, Wang J S, Donovan J L, DeVane C L, Malcolm R, Johnson J A, Youngblood G L, Sweet D H, Langaee T Y and Markowitz J S (2008) Two CES1 gene mutations lead to dysfunctional carboxylesterase 1 activity in man: clinical significance and molecular basis. Am J Hum Genet 82:1241-1248.

1. A method of genotyping a subject for CES1, the method comprising
amplifying a CES1 nucleic acid in a biological sample from the subject by long range polymerase chain reaction (PCR); and
detecting a variant allele of CES1, thereby genotyping the subject for CES1.

2. The method of claim 1, wherein CES1 is an isoform selected from the group consisting of CES1A1, CES1A2, CES1A3, and combinations thereof.

3. The method of claim 1, wherein CES1 is a CES1A1 isoform.

4. The method of claim 1, wherein the variant allele encodes a CES1 polypeptide having reduced or increased carboxylesterase activity or expression relative to a reference.

5. The method of claim 4, wherein the method identifies at least one alteration in an evolutionarily conserved residue of CES1.

6. The method of claim 5, wherein the conserved residue is in a triad residue, in the active site, or in the oxyanion hole.

7. The method of claim 6, wherein the conserved residue serine 221 (S), glutamic acid 354 (E), histidine 468, or Gly141-143.

8. The method of claim 5, wherein the alteration is in a residue that stabilizes substrate-enzyme intermediates or that alters enzyme activity.

9. The method of claim 4, wherein the variant allele is selected from the group consisting of Gly143Glu and Asp260Glu frameshift.

10. The method of claim 4, wherein the variant allele is one or more of an allele set forth in Table 1.

11. The method of claim 1, wherein the variant allele is indicated by the presence of a single nucleotide polymorphism (SNP).

12. The method of claim 11, wherein the SNP is selected from the group consisting of 428G>A and T891del.

13. The method of claim 11, wherein the SNP is one or more of a SNP set forth in Table 1.

14. The method of claim 1, wherein analyzing comprises nucleic acid sequencing, allele-specific hybridization, allele-specific PCR, oligonucleotide microarray analysis, or mass spectrometry.

15-54. (canceled)

55. A method of genotyping a subject, the method comprising:
amplifying a CES1 nucleic acid in a biological sample from the subject by long range polymerase chain reaction (PCR); and
detecting a variant allele of CES1 relative to a wild-type reference sequence, wherein the variant allele encodes a CES1 polypeptide having reduced carboxylesterase activity or expression relative to a reference selected from the group consisting of Gly143Glu and Asp260Glu frameshift.

56. The method of claim 55, wherein the method involves selecting an appropriate drug therapy for the subject, wherein a genotype comprising the presence of Gly143Glu and Asp260Glu frameshift indicates that drug therapy is not appropriate for the subject, and wherein a genotype that is homozygous wild-type indicates that drug therapy is appropriate for the subject.

57. The method of claim 56, wherein the subject is administered an effective amount of the drug administration to the subject is discontinued.

58. The method of claim 55, wherein the variant allele is indicated by the presence of a single nucleotide polymorphism (SNP) selected from the group consisting of 428G>A and T891del.

59. The method of claim 55, wherein analyzing comprises nucleic acid sequencing, allele-specific hybridization, allele-specific PCR, oligonucleotide microarray analysis, or mass spectrometry.

60. A kit for genotyping a CES1 isoform in a subject comprising a set of nucleic acid probes, wherein the nucleic acid probes can selectively bind to and amplify a nucleic acid at a CES1 isoform locus.

61-66. (canceled)
